(19) United States
(12) Patent Application Publication
(10) Pub. No.: US 2024/0376442 A1
(43) Pub. Date: Nov. 14, 2024

(54) CHIMERIC ANTIGEN RECEPTOR POLYPEPTIDES IN COMBINATION WITH TRANS METABOLISM MOLECULES MODULATING KREBS CYCLE AND THERAPEUTIC USES THEREOF

(71) Applicant: SOTIO BIOTECH INC., Boston, MA (US)

(72) Inventors: Kathleen McGinness, Cambridge, MA (US); Seth Ettenberg, Cambridge, MA (US); Luke Barron, Cambridge, MA (US); Michael Fray, Cambridge, MA (US); Charles Wilson, Cambridge, MA (US); Gregory Motz, Cambridge, MA (US)

(73) Assignee: SOTIO BIOTECH INC., Boston, MA (US)

(21) Appl. No.: 18/784,028

(22) Filed: Jul. 25, 2024

Related U.S. Application Data
(63) Continuation of application No. 17/268,345, filed on Feb. 12, 2021, now Pat. No. 12,077,785.
(60) Provisional application No. 62/718,579, filed on Aug. 14, 2018.
(60) Provisional application No. 62/718,491, filed on Aug. 14, 2018.

(51) Int. Cl. C12N 9/04; A61K 35/17; A61K 48/00; A61P 35/00; C07K 14/705; C07K 14/725; C07K 16/30; C12N 9/06; C12N 9/10; C12N 9/80; C12N 9/88

(52) U.S. Cl. CPC C12N 9/0006; A61K 35/17; A61P 35/00; C07K 14/7051; C07K 14/70517; C07K 14/70521; C07K 14/70578; C07K 16/303; C12N 9/0016; C12N 9/1096; C12N 9/80; C12N 9/88; C12Y 101/01037; C12Y 101/01042; C12Y 104/01002; C12Y 206/01001; C12Y 206/01002; C12Y 206/01052; C12Y 305/01002; C12Y 401/01032; A61K 48/00; C07K 2319/02; C07K 2319/03

(57) ABSTRACT

Disclosed herein are genetically engineered hematopoietic cells, which express one or more Krebs cycle modulating polypeptides, and optionally a chimeric receptor polypeptide (e.g., an antibody-coupled T cell receptor (ACTR) polypeptide or a chimeric antigen receptor (CAR) polypeptide) capable of binding to a target antigen of interest. Also disclosed herein are uses of the engineered hematopoietic cells for inhibiting cells expressing a target antigen in a subject in need thereof.

### CROSS REFERENCE TO RELATED APPLICATIONS

This application is a continuation of U.S. patent application Ser. No. 17/268,345, filed Feb. 12, 2021, which is a United States National Phase Application under 35 USC § 371 of International Application No. PCT/US2019/046550, filed Aug. 14, 2019, which claims the benefit of the filing dates of U.S. Provisional Application No. 62/718,491, filed Aug. 14, 2018, and U.S. Provisional Application No. 62/718,579, filed Aug. 14, 2018. The entire contents of each of the prior applications are incorporated by reference herein.

### SEQUENCE LISTING

The application contains a Sequence Listing that has been filed electronically in the form of a compliant XML file, created Jul. 22, 2024, named “063642-513C01US_SeqLst_ST26.xml”, and 177,210 bytes in size, the contents of which are incorporated by reference herein in their entirety.

### BACKGROUND OF DISCLOSURE

Cancer immunotherapy, including cell-based therapy, is used to provoke immune responses attacking tumor cells while sparing normal tissues. It is a promising option for treating various types of cancer because of its potential to evade genetic and cellular mechanisms of drug resistance, and to target tumor cells while sparing normal tissues.

Cell-based therapy may involve cytotoxic T cells having reactivity skewed toward cancer cells. Eshhar et al., Proc. Natl. Acad. Sci. U.S.A.; 1993; 90 (2): 720-724; Geiger et al., J Immunol. 1999; 162 (10): 5931-5939; Brentjens et al., Nat. Med. 2003; 9 (3): 279-286; Cooper et al., Blood. 2003; 101 (4): 1637-1644; and Imai et al., Leukemia. 2004; 18:676-684. One approach is to express a chimeric receptor having an antigen-binding domain fused to one or more T cell activation signaling domains. Binding of a cancer antigen via the antigen-binding domain results in T cell activation and triggers cytotoxicity. Recent results of clinical trials with infusions of chimeric receptor-expressing autologous T lymphocytes provided compelling evidence of their clinical potential. Pule et al., Nat. Med. 2008; 14 (11): 1264-1270; Porter et al., N Engl J Med; 2011; 25; 365 (8): 725-733; Brentjens et al., Blood. 2011; 118 (18): 4817-4828; Till et al., Blood. 2012; 119 (17): 3940-3950; Kochenderfer et al., Blood. 2012; 119 (12): 2709-2720; and Brentjens et al., Sci Transl Med. 2013; 5 (177): 177ra138.

Another approach is to express an antibody-coupled T cell Receptor (ACTR) protein in a hematopoietic cell (e.g., a hematopoietic stem cell, an immune cell, such as an NK cell or a T cell), the ACTR protein containing an extracellular Fc-binding domain. When the ACTR-expressing hematopoietic cells (e.g., ACTR-expressing T cells, also called “ACTR T cells”) are administered to a subject together with an anti-cancer antibody, they may enhance toxicity against cancer cells targeted by the antibody via their binding to the Fc domain of the antibody. Kudo et al., Cancer Research (2014) 74:93-103.

Cell-based immune therapies, while promising, have faced challenges caused by specific characteristics of the tumor microenvironment (TME), which is cellular environment created via the interaction between malignant tumor cells and non-transformed cells. It is therefore of great importance to develop strategies to improve efficacy of cell-based immune therapies in light of the TME.

### SUMMARY OF DISCLOSURE

The present disclosure is based on the development of strategies to modulate the Krebs cycle in hematopoietic cells such as immune cells, including those that express a chimeric receptor polypeptide, such as an antibody-coupled T-cells receptor (ACTR) polypeptide or a chimeric antigen receptor (CAR) polypeptide, for use in cell-based immune therapy. Modulation of the Krebs cycle may be achieved by expressing (e.g., over-expressing) in hematopoietic cells (e.g., hematopoietic stem cells (HSCs) or immune cells such as T cells or natural killer cells) one or more Krebs cycle modulating polypeptides such as those described herein. Such genetically engineered immune cells are expected to have modulated (e.g., enhanced) Krebs cycle reactions, for example, in a low glucose environment, a low-amino acid environment, a low pH environment, and/or a hypoxic environment (e.g., in a tumor microenvironment). Such genetically engineered immune cells may also have modulated epigenetic states (e.g., acetylation states) and/or modulated levels of immunosuppressive metabolites (e.g., kynurenine). As such, hematopoicic cells such as HSCs or immune cells that co-express one or more Krebs cycle modulating polypeptides and a chimeric receptor polypeptide would exhibit superior bioactivities (e.g., under tumor microenvironment such as low glucose, low amino acid, low pH, and/or hypoxic conditions, optionally in the presence of a therapeutic antibody), for example, cell proliferation, activation (e.g., increased cytokine production, e.g., IL-2 or IFNγ production), cytotoxicity, and/or in vivo anti-tumor activity.

Accordingly, provided herein are modified (e.g., genetically modified) hematopoietic cells (e.g., hematopoietic stem cells, immune cells such as T cells or natural killer cells) that have a modulated Krebs cycle relative to a native immune cell of the same type, particularly, for example, in low glucose, low amino acid, low pH, and/or hypoxic conditions. The modified immune cells may express or overly express a Krebs cycle modulating polypeptide.

In some embodiments, the Krebs cycle modulating polypeptide may be an enzyme that catalyzes a reaction of the Krebs cycle. Examples include, but are not limited to, isocitrate dehydrogenase (IDH) such as IDH1 or IDH2, malate dehydrogenase (MDH) such as MDH1 or MDH2, or phosphoglycerate dehydrogenase (PHGDH). In other embodiments, the Krebs cycle modulating polypeptide is an enzyme that uses a Krebs cycle metabolite as a substrate. Examples include, but are not limited to, a glutamic-oxaloacetic transaminase (GOT) such as GOT1 or GOT2 (also known as aspartate transaminase or aspartate aminotransferase) or phosphoenolpyruvate carboxykinase 1 (PCK1). In yet other embodiments, the Krebs cycle modulating polypeptide is an enzyme that converts a precursor to a Krebs cycle metabolite. Examples include, but are not limited to, a phosphoserine aminotransferase (PSAT1), a glutamate dehydrogenase (GDH1), a glutamic-pyruvate transaminase 1 (GPT1), or a glutaminase (GLS).

The modified immune cells may further express a chimeric receptor polypeptide, which may comprise (a) an extracellular target binding domain; (b) a transmembrane domain; and (c) a cytoplasmic signaling domain (e.g., a cytoplasmic domain that comprises an immunoreceptor tyrosine-based activation motif (ITAM)). In some embodiments, the chimeric receptor polypeptide is an antibody-coupled T cell receptor (ACTR), which comprises an extracellular Fc-binding domain (a). In other embodiments, the chimeric receptor is a chimeric antigen receptor (CAR), which comprises an extracellular antigen binding domain (a). In some examples, (c) is located at the C-terminus of the chimeric receptor polypeptide. In some instances, the chimeric polypeptide may further comprise at least one co-stimulatory signaling domain. In other instances, the chimeric receptor polypeptide may be free of co-stimulatory signaling domains.

Any of the chimeric receptor polypeptides described herein (e.g., an ACTR polypeptide or a CAR polypeptide) may further comprise a hinge domain, which is located at the C-terminus of (a) and the N-terminus of (b). In other examples, the chimeric receptor polypeptide may be free of any hinge domain. In yet other examples, the chimeric receptor polypeptide, for example, an ACTR polypeptide, may be free of a hinge domain from any non-CD16A receptor. Alternatively or in addition, the chimeric receptor polypeptide further comprises a signal peptide at its N-terminus.

In some embodiments, the chimeric receptor polypeptide disclosed herein may be an ACTR polypeptide comprising an Fc binding domain (a). In some examples, the Fc binding domain of (a) can be an extracellular ligand-binding domain of an Fc-receptor, for example, an extracellular ligand-binding domain of an Fc-gamma receptor, an Fc-alpha receptor, or an Fc-epsilon receptor. In particular examples, the Fc binding domain is an extracellular ligand-binding domain of CD16A (e.g., F158 CD16A or V158 CD16A), CD32A, or CD64A. In other examples, the Fc binding domain of (a) can be an antibody fragment that binds the Fc portion of an immunoglobulin. For example, the antibody fragment can be a single chain variable fragment (ScFv), a single domain antibody, (e.g., a nanobody). Additionally, the Fc binding domain of (a) can be a naturally-occurring protein that binds the Fc portion of an immunoglobulin or an Fc-binding fragment thereof. For example, the Fc binding domain can be Protein A or Protein G, or an Fc-binding fragment thereof. In further examples, the Fc binding domain of (a) can be a synthetic polypeptide that binds the Fc portion of an immunoglobulin. Examples include, but are not limited to, a Kunitz peptide, a SMIP, an avimer, an affibody, a DARPin, or an anticalin.

In some embodiments, the chimeric receptor polypeptide disclosed herein can be a CAR polypeptide comprising an extracellular antigen binding domain (a). In some examples, the extracellular antigen binding domain of (a) is a single chain antibody fragment that binds to a tumor antigen, a pathogenic antigen, or an immune cell specific to an autoantigen. In certain examples, the tumor antigen is associated with a hematologic tumor. Examples include, but are not limited to, CD19, CD20, CD22, Kappa-chain, CD30, CD123, CD33, LeY, CD138, CD5, BCMA, CD7, CD40, and IL-IRAP. In certain examples, the tumor antigen is associated with a solid tumor. Examples include, but are not limited to, GD2, GPC3, FOLR (e.g., FOLR1 or FOLR2), HER2, EphA2, EFGRVIII, IL13RA2, VEGFR2, ROR1, NKG2D, EpCAM, CEA, Mesothelin, MUC1, CLDN18.2, CD171, CD133, PSCA, cMET, EGFR, PSMA, FAP, CD70, MUC16, LI-CAM, B7H3, and CAIX. In certain examples, the pathogenic antigen is a bacterial antigen, a viral antigen, or a fungal antigen, for example, those described herein.

In some embodiments, the transmembrane domain of (b) in any of the chimeric receptor polypeptide (e.g., ACTR or CAR polypeptide) can be of a single-pass membrane protein, e.g., CD8α, CD8β, 4-1BB, CD28, CD34, CD4, FcεRIγ, CD16A, OX40, CD3ζ, CD3ε, CD3γ, CD3δ, TCRα, CD32, CD64, VEGFR2, FAS, and FGFR2B. Alternatively, the transmembrane domain of (b) can be a non-naturally occurring hydrophobic protein segment.

In some embodiments, the at least one co-stimulatory signaling domain of the chimeric receptor polypeptides described herein (e.g., ACTR or CAR polypeptides), if applicable, can be of a co-stimulatory molecule, which can be 4-1BB, CD28, CD28_{LL→GG} variant, OX40, ICOS, CD27, GITR, ICOS, HVEM, TIM1, LFA1, and CD2. In some examples, the at least one co-stimulatory signaling domains is a CD28 co-stimulatory signaling domain or a 4-1BB co-stimulatory signaling domain. In some instances, the ACTR polypeptide may comprise two co-stimulatory signaling domains. In some instances, one of the co-stimulatory signaling domains is a CD28 co-stimulatory signaling domain; and the other co-stimulatory domain can be a 4-1BB co-stimulatory signaling domain, an OX40 co-stimulatory signaling domain, a CD27 co-stimulatory signaling domain, or an ICOS co-stimulatory signaling domain. Specific examples include, but are not limited to, CD28 and 4-1BB; or CD28_{LL→GG} variant and 4-1BB. Alternatively, any of the chimeric receptor polypeptie may be free of any co-stimulatory signaling domain.

In some embodiments, the cytoplasmic signaling domain of (c) in any of the chimeric receptor polypeptides described herein (e.g., ACTR or CAR polypeptides) can be a cytoplasmic domain of CD3ζ or FcεRIγ.

In some embodiments, the hinge domain of any of the chimeric polypeptides described herein (e.g., ACTR or CAR polypeptides), when applicable, can be of CD28, CD16A, CD8α, or IgG. In other examples, the hinge domain is a non-naturally occurring peptide. For example, the non-naturally occurring peptide may be an extended recombinant polypeptide (XTEN) or a (Gly4Ser)ₙ polypeptide, in which n is an integer of 3-12, inclusive. In some examples, the hinge domain is a short segment, which may contain up to 60 amino acid residues.

In specific examples, an ACTR polypeptide as described herein may comprise (i) a CD28 co-stimulatory domain; and (ii) a CD28 transmembrane domain, a CD28 hinge domain, or a combination thereof. For example, the ACTR polypeptide comprises components (a)-(c) as shown in Table 3. In particular examples, the ACTR polypeptide comprises the amino acid sequence selected from SEQ ID NOs: 1-80.

In specific examples, a CAR polypeptide described herein may comprise (i) a CD28 co-stimulatory domain or a 4-1BB co-stimulatory domain; and (ii) a CD28 transmembrane domain, a CD28 hinge domain, or a combination thereof. In further specific examples, a CAR polypeptide described herein may comprise (i) a CD28 co-stimulatory domain or a 4-1BB co-stimulatory domain, (ii) a CD8 transmembrane domain, a CD8 hinge domain, or a combination thereof. For example, the CAR polypeptide may comprise an amino acid sequence selected from SEQ ID NOs: 104 and 105.

The hematopoietic cells described herein, expressing the Krebs cycle modulating polypeptide and optionally the chimeric receptor polypeptide, may be a hematopoietic stem cell or a progeny thereof. In some embodiments, the hematopoietic cells can be immune cells such as natural killer cell, monocyte/macrophage, neutrophil, cosinophil, or T cell. The immune cells can be derived from peripheral blood mononuclear cells (PBMC), hematopoietic stem cells (HSCs), or induced pluripotent stem cells (iPSCs). In some examples, the immune cell is a T cell, in which the expression of an endogenous T cell receptor, an endogenous major histocompatibility complex, an endogenous beta-2-microglobulin, or a combination thereof has been inhibited or eliminated.

Any of the hematopoietic cells (e.g., HSCs or immune cells) described herein may comprise a nucleic acid or a nucleic acid set, which collectively comprises: (a) a first nucleotide sequence encoding the Krebs cycle modulating polypeptide; and optionally (b) a second nucleotide sequence encoding the chimeric antigen receptor (CAR) polypeptide. The nucleic acid or the nucleic acid set is an RNA molecule or a set of RNA molecules. In some instances, the immune cell comprises the nucleic acid, which comprises both the first nucleotide sequence and the second nucleotide sequence. In some embodiments, the coding sequence of the Krebs cycle modulating polypeptide is upstream of that of the CAR polypeptide. In some embodiments, the coding sequence of the CAR polypeptide is upstream of that of the Krebs cycle modulating polypeptide. Such a nucleic acid may further comprise a third nucleotide sequence located between the first nucleotide sequence and the second nucleotide sequence, wherein the third nucleotide sequence encodes a ribosomal skipping site (e.g., a P2A peptide), an internal ribosome entry site (IRES), or a second promoter.

In some examples, the nucleic acid or the nucleic acid set is comprised within a vector or a set of vectors, which can be an expression vector or a set of expression vectors (e.g., viral vectors such as lentiviral vectors or retroviral vectors). A nucleic acid set or a vector set refers to a group of two or more nucleic acid molecules or two or more vectors, each encoding one of the polypeptides of interest (i.e., the Krebs cycle modulating polypeptide and the CAR polypeptide). Any of the nucleic acids described herein is also within the scope of the present disclosure.

In another aspect, the present disclosure provides a pharmaceutical composition, comprising any of the immune cells described herein and a pharmaceutically acceptable carrier.

Moreover, provided herein is a method for inhibiting cells expressing a target antigen (e.g., reducing the number of such cells, blocking cell proliferation, and/or suppressing cell activity) in a subject, the method comprising administering to a subject in need thereof a population of the immune cells described herein, which may co-express the Krebs cycle modulating polypeptide and the CAR polypeptide. The subject (e.g., a human patient such as a human patient suffering from a cancer) may have been treated or is being treated with an anti-cancer therapy (e.g., an anti-cancer agent). In some examples, at least some of the cells expressing the target antigen are located in a low-glucose environment, a low-amino acid (e.g., low glutamine) environment, a low-pH environment, and/or a hypoxic environment, for example a tumor microenvironment.

In some examples, the immune cells are autologous. In other examples, the immune cells are allogeneic. In any of the methods described herein, the immune cells can be activated, expanded, or both ex vivo. In some instances, the immune cells comprise T cells, which are activated in the presence of one or more of anti-CD3 antibody, anti-CD28 antibody, IL-2, phytohemoagglutinin, and an engineered artificial stimulatory cell or particle. In other instances, the immune cells comprise natural killer cells, which are activated in the presence of one or more of 4-1BB ligand, anti-4-1BB antibody, IL-15, anti-IL-15 receptor antibody, IL-2, IL-12, IL-21 and K562 cells, an engineered artificial stimulatory cell or particle.

In some examples, the subject to be treated by the methods described herein may be a human patient suffering from a cancer, for example, carcinoma, lymphoma, sarcoma, blastoma, and leukemia. Additional exemplary target cancer includes, but are not limited to, a cancer of B-cell origin, breast cancer, gastric cancer, neuroblastoma, osteosarcoma, lung cancer, skin cancer, prostate cancer, colon cancer, renal cell carcinoma, ovarian cancer, rhabdomyosarcoma, leukemia, mesothelioma, pancreatic cancer, head and neck cancer, retinoblastoma, glioma, glioblastoma, liver cancer, and thyroid cancer. Exemplary cancers of B-cell origin is selected from the group consisting of B-lineage acute lymphoblastic leukemia, B-cell chronic lymphocytic leukemia, and B-cell non-Hodgkin's lymphoma.

Also within the scope of the present disclosure are uses of the genetically engineered immune cells described herein, which co-express a Krebs cycle modulating polypeptide and a CAR polypeptide for treating a target disease or disorder such as cancer or an infectious disorder, and uses thereof for manufacturing a medicament for the intended medical treatment.

The details of one or more embodiments of the disclosure are set forth in the description below. Other features or advantages of the present disclosure will be apparent from the detailed description of several embodiments and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a diagram showing proliferation of T cells expressing a GPC3-targeting CAR (SEQ ID NO: 104) or “mock” untransduced T cells in the presence of GPC3-expressing JHH7 or Hep3B target cells and varying concentrations of glucose. Proliferation of CAR-T cells varies as a function of glucose concentration.

FIG. 2A and FIG. 2B are diagrams that show proliferation of T cells expressing a GPC3-targeting CAR (SEQ ID NO: 104) or co-expressing the GPC3-targeting CAR and GOT1 (SEQ ID NO: 87) in the presence of GPC3-expressing JHH7 target cells. FIG. 2A: 1.25 mM glucose. FIG. 2B: 10 mM glucose. Proliferation of CAR-T cells co-expressing GOT1 is enhanced relative to cells expressing CAR-T alone at both 1.25 mM and 10 mM glucose.

FIG. 3A and FIG. 3B are diagrams that show proliferation of T cells expressing a GPC3-targeting CAR (SEQ ID NO: 104) or co-expressing the GPC3-targeting CAR and GOT2 (SEQ ID NO: 88) in the presence of GPC3-expressing JHH7 target cells. FIG. 3A: 1.25 mM glucose. FIG. 3B: 10 mM glucose. Proliferation of CAR-T cells co-expressing GOT2 is enhanced relative to cells expressing CAR-T alone at both 1.25 mM and 10 mM glucose.

FIG. 4 is a chart showing JHH7 xenograft tumor inhibition activity of T cells expressing a GPC3-targeting CAR and T cells co-expressing the GPC3-targeting CAR (SEQ ID NO:104) and GOT2 (SEQ ID NO:88).

FIG. 5 is a chart showing NCI-H446 xenograft tumor inhibition activity of T cells expressing a GPC3-targeting CAR and T cells co-expressing the GPC3-targeting CAR (SEQ ID NO: 104) and GOT2 (SEQ ID NO: 88).

FIG. 6 is a chart showing Hep3B xenograft tumor inhibition activity of T cells expressing a GPC3-targeting CAR and T cells co-expressing the GPC3-targeting CAR (SEQ ID NO: 104) and GOT2 (SEQ ID NO: 88).

FIG. 7 is a chart showing peripheral blood counts of T cells from mice bearing Hep3B xenograft tumors treated with T cells expressing a GPC3-targeting CAR and T cells co-expressing the GPC3-targeting CAR (SEQ ID NO: 104) and GOT2 (SEQ ID NO: 88).

FIG. 8A and FIG. 8B show protein expression and aminotransferase activity in T cells co-expressing anti-GPC3 CAR and GOT2. FIG. 8A: Western blot demonstrating increased expression of GOT2 in T cells co-expressing anti-GPC3 CAR (SEQ ID NO: 104) and GOT2 (SEQ ID NO: 88) relative to T cells expressing CAR alone. T cells were activated with Hep3B GPC3-expressing tumor cells for 4 days. Blots were stained with anti-human GOT2 antibody or anti-human GAPDH for loading control, respectively. FIG. 8B: T cells co-expressing anti-GPC3 CAR and GOT2 show increased aspartate aminotransferase (AST) enzyme activity relative to T cells expressing CAR alone. T cells were activated with Hep3B GPC3-expressing tumor cells for 8 days and aspartate aminotransferase enzyme activity was measured using a commercially available Aspartate Aminotransferase Activity Assay (Abcam).

FIG. 9A and FIG. 9B are diagrams that show T cells co-expressing GOT2 (SEQ ID NO: 88) and anti-GPC3 CAR (SEQ ID NO: 104) or ACTR (SEQ ID NO: 1) show enhanced proliferation relative to T cells expressing CAR or ACTR alone. FIG. 9A: CAR T cells were activated with Hep3B GPC3-expressing tumor cells for 6 days. Proliferation was assessed by flow cytometry using intracellular cell trace violet dilution as a measure of cell division for CD3+ T cells.; FIG. 9B: ACTR T cells were activated with HepG2 GPC3-expressing tumor cells plus anti-GPC3 antibody (GC33, 1 μg/ml) for 3 days. Proliferation was assessed by flow cytometry cell counts of CD3+ T cells. Results are expressed for GOT2 co-expressing constructs relative to parental construct.

FIGS. 10A-10C are diagrams that show T cells co-expressing GOT2 (SEQ ID NO: 88) and anti-GPC3 CAR (SEQ ID NO: 104) or ACTR (SEQ ID NO: 1 and SEQ ID NO: 57) show enhanced IL-17A production relative to T cells expressing CAR or ACTR alone. FIG. 10A: GPC3-CAR (SEQ ID NO: 104). FIG. 10B: ACTR (SEQ ID NO: 1). FIG. 10C: ACTR (SEQ ID NO: 57). T cells were activated for 24 hrs with GPC3-expressing HepG2 cells with the addition of anti-GPC3 antibody to ACTR co-cultures, IL-17A was measured in supernatants using an MSD assay. Enhanced IL-17A production relative to T cells expressing CAR or ACTR alone was observed for constructs co-expressing GOT2, independent of construct type (CAR or ACTR) and primary costimulatory domain (4-1BB or CD28).

FIGS. 11A-11C are diagrams that show T cells co-expressing GOT2 (SEQ ID NO: 88) and anti-GPC3 CAR (SEQ ID NO: 104) or ACTR (SEQ ID NO: 1 and SEQ ID NO: 57) show enhanced CD4+ polyfunctionality relative to T cells expressing CAR or ACTR alone. FIG. 11A: GPC3-CAR (SEQ ID NO: 104). FIG. 11B: ACTR (SEQ ID NO: 1). FIG. 11C: ACTR (SEQ ID NO: 57). T cells were incubated at 37° C., 5% CO₂ for 6 hr with GPC3-expressing HepG2 cells, with the additions of anti-GPC3 antibody in ACTR co-cultures, in the presence of protein transport inhibitors Brefeldin A and Monensin. Cells were fixed and intracellular staining for IFNγ, IL-2, TNFα, and IL-17A was evaluated by flow cytometry. A higher frequency of CD4+ T cells producing greater than one, two, or three cytokines simultaneously was observed for T cells co-expressing GOT2 and ACTR or CAR relative to cells expressing CAR or ACTR alone, independent of chimeric receptor type (CAR or ACTR) and primary costimulatory domain (4-1BB or CD28).

FIGS. 12A-12C are diagrams that show T cells co-expressing GOT2 (SEQ ID NO: 88) and anti-GPC3 CAR (SEQ ID NO: 104) or ACTR (SEQ ID NO: 1 and SEQ ID NO: 57) showed a greater population of less-differentiated, naïve-like CD8+ T cells relative to T cells expressing CAR or ACTR alone. FIG. 12A: GPC3-CAR (SEQ ID NO: 104). FIG. 12B: ACTR (SEQ ID NO: 1). FIG. 12C: ACTR (SEQ ID NO: 57). CAR (SEQ ID NO: 104) T cells generated from 11 healthy donors and ACTR T cells generated from 2 healthy donors (4-1BB primary costimulation domain, SEQ ID NO: 1; CD28 primary costimulation domain, SEQ ID NO: 57) were stained for a panel of surface markers and analyzed by flow cytometry for naïve-like phenotypic markers. Cells were gated on CD8+/CAR+ (CAR) or CD8+/CD16+ (ACTR+). The subset of these populations staining positive for markers CD27, CD45RO and CD62L and staining negative for CD45RA was enriched in T cells co-expressing GOT2 and ACTR or CAR relative to cells expressing CAR or ACTR alone, independent of chimeric receptor type (CAR or ACTR) and primary costimulatory domain (4-1BB or CD28).

FIG. 13 is a diagram that shows T cells co-expressing GOT2 (SEQ ID NO: 88) and anti-GPC3 CAR (SEQ ID NO: 104) demonstrated a less-differentiated (younger) CD8+ T cell phenotype relative to T cells expressing CAR alone. CAR T cells generated from 11 healthy donors were stained for a panel of surface markers and analyzed by flow cytometry. Cells were gated on CD8+/CAR+. The populations staining CD27+CD45RO+, CD27+RO−, CD27+CD45RA+ or CD27+CD45RA-indicate a less-differentiated phenotype. These subsets were enriched in CAR T cells co-expressing GOT2 relative to T cells expressing CAR alone.

FIG. 14 is a diagram that shows T cells co-expressing GOT2 (SEQ ID NO: 88) and anti-GPC3 CAR (SEQ ID NO: 104) demonstrated a proliferative advantage relative to T cells expressing CAR alone under chronic antigen stimulation and hypoxia. CAR T cells were activated with Hep3B GPC3-expressing tumor cells for 3 days; and then re-stimulated with fresh target cells for 3 days under normoxic (20% oxygen) or hypoxic (1.5%) conditions to simulate the tumor microenvironment stress of chronic antigen stimulation with and without the additional stress of hypoxia. Proliferation was then assessed by flow cytometry of CD3+ T cells using intracellular cell trace violet dilution as a measure of cell division and the inverse of mean fluorescence is plotted.

FIG. 15 is a diagram that shows T cells co-expressing GOT2 (SEQ ID NO: 88) and anti-GPC3 CAR (SEQ ID NO: 104) demonstrated a proliferative advantage relative to T cells expressing CAR alone under limiting glucose. CAR T cells were activated with HepG2 GPC3-expressing tumor cells in the presence of 10 mM or 1.25 mM glucose for 7 days. Proliferation was then assessed by flow cytometry using intracellular cell trace violet dilution as a measure of cell division and the inverse of mean fluorescence is plotted. CARs expressing GOT2 had a proliferative advantage in under both glucose conditions.

FIG. 16 is a diagram that shows T cells co-expressing GOT2 (SEQ ID NO: 88) and ACTR (SEQ ID NO: 57) demonstrated enhanced proliferation in the presence of the T cell inhibitor kynurenine relative to T cells expressing ACTR alone.

FIGS. 17A-17C are diagrams that show T cells co-expressing GOT2 (SEQ ID NO: 88) and anti-GPC3 CAR (SEQ ID NO: 104) demonstrated enhanced activation in tumor relative to T cells expressing CAR alone. FIG. 17A: CD69. FIG. 17B: CD25.

FIG. 17C: ICOS. T cells co-expressing CAR and GOT2 demonstrated increased expression of activation markers relative to T cells expressing CAR alone.

FIG. 18A and FIG. 18B are diagrams that show T cells co-expressing GOT2 (SEQ ID NO: 88) and anti-GPC3 CAR (SEQ ID NO: 104) demonstrated greater resistance to exhaustion in tumors relative to T cells expressing CAR alone. FIG. 18A: CD4+ T cell subsct. FIG. 18B: CD8+ T cell subset.

FIGS. 19A-19D are diagrams that show CARs co-expressing GOT2 resist exhaustion in GPC3-expressing tumor; activation of CARs is specific to tumor vs. antigen-negative spleen. FIG. 19A: day 6, CD4+ T cell subset. FIG. 19B: day 6, CD8+ T cell subset. FIG. 19C: day 13, CD4+ T cell subset. FIG. 19D: day 13, CD8+ T cell subset.

FIG. 20A and FIG. 20B are diagrams that show T cells co-expressing GOT2 (SEQ ID NO: 88) and anti-GPC3 CAR (SEQ ID NO: 104) demonstrate greater function after exposure to tumors in vivo relative to T cells expressing CAR alone. FIG. 20A: IL-17A. FIG. 20B: IFNγ.

### DETAILED DESCRIPTION OF DISCLOSURE

Tumor microenvironments have specific characteristics, such as low glucose, low amino acid, low pH, and/or hypoxic conditions, some of which may constrain the activity of effector immune cells such as effector T cells. The present disclosure is based, at least in part, on the development of various approaches for enhancing effector immune cell activities in tumor microenvironments via modulating (e.g., enhancing) Krebs cycle reactions by the effector immune cells, thereby enhancing their growth and bioactivity. The Krebs cycle can be modulated by various factors, including the expression level of Krebs cycle enzymes (e.g., naturally occurring enzymes or functional equivalents thereof), the activation status of such enzymes, the expression level and activity of enzymes that uses Krebs cycle metabolites as substrates, and/or the expression level and activity of enzymes that converts precursors to Krebs cycle metabolites.

The studies disclosed herein demonstrate, unexpectedly, that co-expression of a Krebs cycle modulating polypeptide such as GOT1 or GOT2 and a chimeric receptor polypeptide such as a CAR (e.g., having a 4-1BB co-stimulatory domain) or an ACTR (e.g., having a 4-1BB or CD28 co-stimulatory domain) in immune cells such as T cells exhibited superior features both in vitro and in vivo as relative to immune cells expressing only the CAR or the ACTR. For example, co-expression of GOT1 or GOT2 with CAR or ACTR enhanced T cell proliferation/expansion and T cell function, particularly under solid tumor microenvironment conditions (e.g., hypoxia, low glucose condition, and presence of TME inhibitors). For example, co-expression of GOT2 contributed to resistance of long-term expression of inhibitory receptors (e.g., PD1) and maintained T cell function in the tumor microenvironment. Further, co-expression of GOT1 or GOT2 with CAR or ACTR enhanced anti-tumor effects.

Accordingly, the present disclosure provides modified (e.g., genetically engineered) hematopoictic cells (e.g., HSCs or immune cells) that possess elevated Krebs cycle modulating activity. Modification of the Krebs cycle in immune cells can be achieved by any suitable approaches. For example, the modified immune cells may express one or more Krebs cycle modulating factors, which can be a molecule of any type that either is involved in the Krebs cycle directly (e.g., an enzyme catalyzing one or more reactions of the Krebs cycle) or modulates the Krebs cycle indirectly via affecting the expression level, activity, and/or degradation of one or more Krebs cycle enzymes. In some embodiments, the Krebs cycle modulating factor can be a Krebs cycle modulating polypeptide as those described herein that enhances Krebs cycle modulation in immune cells expressing such as relative to their native counterpart. In other embodiments, the Krebs cycle modulating factor can be a nucleic acid (e.g., microRNA, interfering RNA such as siRNA or shRNA, or antisense nucleic acid) that regulates expression of one or more enzymes catalyzing one or more reactions of the Krebs cycle. In further embodiments, the Krebs cycle modulating factor may be a transcriptional factor that regulates expressing of one or more Krebs cycle enzymes.

Such a genetically engineered immune cell may further express a chimeric receptor polypeptide, e.g., an antibody-coupled T cell receptor (ACTR) polypeptide or a chimeric antigen receptor (CAR) polypeptide. Also provided herein are uses of the genetically engineered immune cells, optionally in combination with an Fc-containing agent when needed (e.g., when the immune cells express an ACTR polypeptide), for improving immune cell proliferation, and/or an inhibiting or decreasing in target cells (e.g., target cancer cells) in a subject (e.g., a human cancer patient), e.g., via ADCC. The present disclosure also provides pharmaceutical compositions and kits comprising the described genetically engineered immune cells.

The genetically engineered immune cells described herein, expressing (e.g., over-expressing) a Krebs cycle modulating polypeptide, may confer at least the following advantages. The expression of the Krebs cycle modulating polypeptide would enhance the metabolic activity of a T cell. As such, the genetically engineered immune cells may proliferate better, produce more cytokines, exhibit greater anti-tumor cytotoxicity, exhibit less immunosuppressive metabolites, and/or exhibit greater T cell survival in a tumor environment (e.g., low-glucose, low amino acid, low pH, and/or hypoxic environment relative to immune cells that do not express (or do not over-express) the Krebs cycle modulating polypeptide, leading to enhanced cytokine production, survival rate, cytotoxicity, and/or anti-tumor activity.

### I. Krebs Cycle Modulating Polypeptides

As used herein, a Krebs cycle modulating polypeptide refers to any polypeptide that modulates the Krebs cycle, which links various metabolic pathways such as the metabolic pathways for processing glucose, amino acids and/or fatty acids. The Krebs cycle, also known as the citric acid cycle and tricarboxylic acid (TCA) cycle, is a metabolic pathway which begins with adding acetyl-CoA generated in glycolysis to oxaloacetate, forming citrate. Such a polypeptide may regulate one or more reversible enzymatic reactions in the Krebs cycle in favor of one direction relative to the other so as to regulate the metabolites generated therefrom. An increased input to the Krebs cycle from one metabolic source may redirect upstream metabolites of another pathway. For example, a Krebs cycle modulating polypeptide may redirect alanine into the glycolytic pathway, increase α-keto-glutarate (αKG) for TCA and/or serine synthesis pathway via, e.g., PSAT1, facilitate the TCA cycle in mitochondria and/or the cytoplasm; move glucose out of the TCA cycle to produce serine via PSAT1 or PSPH.

Additionally, a Krebs cycle modulating polypeptide may increase Krebs cycle activity in the mitochondria and/or the cytoplasm. A Krebs cycle modulating polypeptide may also transform inhibitory metabolites such as lactate into pyruvate to promote Krebs cycle activity. Any such Krebs cycle modulating polypeptide, which may be of any suitable species (e.g., mammalian such as human), may be contemplated for use with the compositions and methods described herein.

Alternatively, the Krebs cycle metabolite polypeptide may be a molecule that is mutated to mimic an activated Krebs cycle modulating polypeptide (e.g., a phosphorylation mimic) or mutated to impact its intracellular trafficking (e.g., traffic to mitochondria) such that the activity of the Krebs cycle is modulated. Alternatively, expression of an endogenous Krebs cycle polypeptide may be modulated, for example, by expressing a transcription factor or a microRNA, or by modulating the polypeptide's stability or degradation, for example, by modulating factors that mediate its degradation, for example an E3 ligase that is part of the ubiquitin/proteasome pathway. Additionally, the trafficking of an endogenous Krebs cycle polypeptide may be modulated, for example, by expressing a polypeptide that increases its trafficking to a desired subcellular compartment, for example, mitochondria. Further, a Krebs cycle modulating polypeptide may be a polypeptide that enzymatically converts substrates, found in high levels in the tumor microenvironment, that inhibit or limit the activity of immune cells, to molecules that no longer have inhibitory effects thus improving immune cell function.

Krebs cycle modulating polypeptides may be polypeptides having the enzymatic activity to catalyze a reaction in the Krebs cycle, for example, naturally-occurring enzymes that catalyze a Krebs cycle reaction, or functional variants/homologs thereof that catalyze the same Krebs cycle reaction. Examples include, but are not limited to, isocitrate dehydrogenase (IDH, including IDH1 or IDH2), malate dehydrogenase (MDH, including MDH1 or MDH2), or phosphoglycerate dehydrogenase (PHGDH).

Isocitrate dehydrogenase (IDH) refers to any polypeptide (enzyme) that catalyzes the oxidative decarboxylation of isocitrate, leading to production of α-ketoglutarate and CO2. Malate dehydrogenase (MDH) refers to any polypeptide (enzyme) that catalyzes the oxidation of malate to oxaloacetate using the reduction of NAD⁺ to NADH. Phosphoglycerate dehydrogenase (PHGDH) refers to any polypeptide (enzyme) that catalyzes the reactions to convert 3-phospho-D-glycerate to 3-phosphonooxypyruvate and to convert 2-hydrozyglutarate to 2-oxoglutarate, using the reduction of NAD⁺ to NADH. Amino acid sequences of exemplary IDH, MDH, and PHGDH are provided below:

IDH1
(SEQ ID NO: 81)
MSKKISGGSVVEMQGDEMTRIIWELIKEKLIFPYVELDLHSYDLGIENRDATNDQVTKDAAE
AIKKHNVGVKCATITPDEKRVEEFKLKQMWKSPNGTIRNILGGTVFREAIICKNIPRLVSGWV
KPIIIGRHAYGDQYRATDFVVPGPGKVEITYTPSDGTQKVTYLVHNFEEGGGVAMGMYNQD
KSIEDFAHSSFQMALSKGWPLYLSTKNTILKKYDGRFKDIFQEIYDKQYKSQFEAQKIWYEHR
LIDDMVAQAMKSEGGFIWACKNYDGDVQSDSVAQGYGSLGMMTSVLVCPDGKTVEAEAA
HGTVTRHYRMYQKGQETSTNPIASIFAWTRGLAHRAKLDNNKELAFFANALEEVSIETIEAGF
MTKDLAACIKGLPNVQRSDYLNTFEFMDKLGENLKIKLAQAKL
IDH2
(SEQ ID NO: 82)
MAGYLRVVRSLCRASGSRPAWAPAALTAPTSQEQPRRHYADKRIKVAKPVVEMDGDEMTRI
IWQFIKEKLILPHVDIQLKYFDLGLPNRDQTDDQVTIDSALATQKYSVAVKCATITPDEARVE
EFKLKKMWKSPNGTIRNILGGTVFREPIICKNIPRLVPGWTKPITIGRHAHGDQYKATDFVAD
RAGTFKMVFTPKDGSGVKEWEVYNFPAGGVGMGMYNTDESISGFAHSCFQYAIQKKWPLY
MSTKNTILKAYDGRFKDIFQEIFDKHYKTDFDKNKIWYEHRLIDDMVAQVLKSSGGFVWAC
KNYDGDVQSDILAQGFGSLGLMTSVLVCPDGKTIEAEAAHGTVTRHYREHQKGRPTSTNPIA
SIFAWTRGLEHRGKLDGNQDLIRFAQMLEKVCVETVESGAMTKDLAGCIHGLSNVKLNEHF
LNTTDFLDTIKSNLDRALGRQ
MDH1
(SEQ ID NO: 83)
MSEPIRVLVTGAAGQIAYSLLYSIGNGSVFGKDQPIILVLLDITPMMGVLDGVLMELQDCALP
LLKDVIATDKEDVAFKDLDVAILVGSMPRREGMERKDLLKANVKIFKSQGAALDKYAKKSV
KVIVVGNPANTNCLTASKSAPSIPKENFSCLTRLDHNRAKAQIALKLGVTANDVKNVIIWGN
HSSTQYPDVNHAKVKLQGKEVGVYEALKDDSWLKGEFVTTVQQRGAAVIKARKLSSAMSA
AKAICDHVRDIWFGTPEGEFVSMGVISDGNSYGVPDDLLYSFPVVIKNKTWKFVEGLPINDFS
REKMDLTAKELTEEKESAFEFLSSA
MDH2
(SEQ ID NO: 84)
MLSALARPASAALRRSFSTSAQNNAKVAVLGASGGIGQPLSLLLKNSPLVSRLTLYDIAHTPG
VAADLSHIETKAAVKGYLGPEQLPDCLKGCDVVVIPAGVPRKPGMTRDDLFNTNATIVATLT
AACAQHCPEAMICVIANPVNSTIPITAEVFKKHGVYNPNKIFGVTTLDIVRANTFVAELKGLD
PARVNVPVIGGHAGKTIIPLISQCTPKVDFPQDQLTALTGRIQEAGTEVVKAKAGAGSATLSM
AYAGARFVFSLVDAMNGKEGVVECSFVKSQETECTYFSTPLLLGKKGIEKNLGIGKVSSFEE
KMISDAIPELKASIKKGEDFVKTLK
PHGDH
(SEQ ID NO: 85)
MAFANLRKVLISDSLDPCCRKILQDGGLQVVEKQNLSKEELIAELQDCEGLIVRSATKVTADV
INAAEKLQVVGRAGTGVDNVDLEAATRKGILVMNTPNGNSLSAAELTCGMIMCLARQIPQA
TASMKDGKWERKKFMGTELNGKTLGILGLGRIGREVATRMQSFGMKTIGYDPIISPEVSASF
GVQQLPLEEIWPLCDFITVHTPLLPSTTGLLNDNTFAQCKKGVRVVNCARGGIVDEGALLRA
LQSGQCAGAALDVFTEEPPRDRALVDHENVISCPHLGASTKEAQSRCGEEIAVQFVDMVKGK
SLTGVVNAQALTSAFSPHTKPWIGLAEALGTLMRAWAGSPKGTIQVITQGTSLKNAGNCLSP
AVIVGLLKEASKQADVNLVNAKLLVKEAGLNVTTSHSPAAPGEQGFGECLLAVALAGAPYQ
AVGLVQGTTPVLQGLNGAVFRPEVPLRRDLPLLLFRTQTSDPAMLPTMIGLLAEAGVRLLSY
QTSLVSDGETWHVMGISSLLPSLEAWKQHVTEAFQFHF

Krebs cycle modulating polypeptides may also be polypeptides that uses a Krebs cycle metabolite as a substrate, for example, a glutamic-oxaloacetic transaminase (GOT, including GOT1 and GOT2) or phosphoenolpyruvate carboxykinase 1 (PCK1). Glutamic-oxaloacetic transaminase (GOT), also known as aspartate aminotransferase (AST), refers to any pyridoxal phosphate (PLP)-dependent enzyme that catalyzes the reversible reaction to transfer an α-amino group between aspartate and glutamate. In some instances, GOT exists in cytoplasmic and inner-membrane mitochondrial forms, GOT1 and GOT2, respectively. GOT is also known as. Various PLP-depending enzymes, in addition to GOT1 and GOT2, may also be Krebs cycle modulating polypeptides, for example, aminotransferase, tryptophan synthase, alanine racemase, D-amino acid aminotransferase, and glycogen phophorylase. All of these enzymes are within the scope of the present disclosure. Phosphoenolpyruvate carboxykinase 1 (PCK1) refers to any polypeptide (enzyme) that converts oxaloacetate into phosphoenolpyruvate and carbon dioxide. Amino acid sequences of exemplary GOT and PCK1 are provided below:

PCK1
(SEQ ID NO: 86)
MPPQLQNGLNLSAKVVQGSLDSLPQAVREFLENNAELCQPDHIHICDGSEEENGRLLGQMEE
EGILRRLKKYDNCWLALTDPRDVARIESKTVIVTQEQRDTVPIPKTGLSQLGRWMSEEDFEK
AFNARFPGCMKGRTMYVIPFSMGPLGSPLSKIGIELTDSPYVVASMRIMTRMGTPVLEAVGD
GEFVKCLHSVGCPLPLQKPLVNNWPCNPELTLIAHLPDRREIISFGSGYGGNSLLGKKCFALR
MASRLAKEEGWLAEHMLILGITNPEGEKKYLAAAFPSACGKTNLAMMNPSLPGWKVECVG
DDIAWMKFDAQGHLRAINPENGFFGVAPGTSVKTNPNAIKTIQKNTIFTNVAETSDGGVYWE
GIDEPLASGVTITSWKNKEWSSEDGEPCAHPNSRFCTPASQCPIIDAAWESPEGVPIEGIIFGGR
RPAGVPLVYEALSWQHGVFVGAAMRSEATAAAEHKGKIIMHDPFAMRPFFGYNFGKYLAH
WLSMAQHPAAKLPKIFHVNWFRKDKEGKFLWPGFGENSRVLEWMFNRIDGKASTKLTPIGY
IPKEDALNLKGLGHINMMELFSISKEFWEKEVEDIEKYLEDQVNADLPCEIEREILALKQRISQ
M
GOT1
(SEQ ID NO: 87)
MAPPSVFAEVPQAQPVLVFKLTADFREDPDPRKVNLGVGAYRTDDCHPWVLPVVKKVEQKI
ANDNSLNHEYLPILGLAEFRSCASRLALGDDSPALKEKRVGGVQSLGGTGALRIGADFLARW
YNGTNNKNTPVYVSSPTWENHNAVFSAAGFKDIRSYRYWDAEKRGLDLQGFLNDLENAPEF
SIVVLHACAHNPTGIDPTPEQWKQIASVMKHRFLFPFFDSAYQGFASGNLERDAWAIRYFVSE
GFEFFCAQSFSKNFGLYNERVGNLTVVGKEPESILQVLSQMEKIVRITWSNPPAQGARIVAST
LSNPELFEEWTGNVKTMADRILTMRSELRARLEALKTPGTWNHITDQIGMFSFTGLNPKQVE
YLVNEKHIYLLPSGRINVSGLTTKNLDYVATSIHEAVTKIQ
GOT2
(SEQ ID NO: 88)
MALLHSGRVLPGIAAAFHPGLAAAASARASSWWTHVEMGPPDPILGVTEAFKRDTNSKKMN
LGVGAYRDDNGKPYVLPSVRKAEAQIAAKNLDKEYLPIGGLAEFCKASAELALGENSEVLKS
GRFVTVQTISGTGALRIGASFLQRFFKFSRDVFLPKPTWGNHTPIFRDAGMQLQGYRYYDPKT
CGFDFTGAVEDISKIPEQSVLLLHACAHNPTGVDPRPEQWKEIATVVKKRNLFAFFDMAYQG
FASGDGDKDAWAVRHFIEQGINVCLCQSYAKNMGLYGERVGAFTMVCKDADEAKRVESQL
KILIRPMYSNPPLNGARIAAAILNTPDLRKQWLQEVKVMADRIIGMRTQLVSNLKKEGSTHN
WQHITDQIGMFCFTGLKPEQVERLIKEFSIYMTKDGRISVAGVTSSNVGYLAHAIHQVTK

In addition, a Krebs cycle modulating polypeptide may be an enzyme that converts a precursor to a Krebs cycle metabolite, for example, phosphoserine aminotransferase (PSAT1), glutamate dehydrogenase (GDH1; also known as GLUD1), glutamate-pyruvate transaminase 1 (GPT1), or glutaminase (GLS). Phosphoserine aminotransferase (PSAT1) refers to any polypeptide (enzyme) that catalyzes the reversible conversion of 3-phosphohydroxypyruvate to phosphoserine and of 3-hydroxy-2-oxo-4-phosphonooxybutanoate to phosphohydroxythreonine. PSAT1 makes 2-oxoglutarate and O-phospho-L-serine. Glutamate dehydrogenase (GDH1) refers to any polypeptide (enzyme) that converts glutamate to α-ketoglutarate, and vice versa. GDH1 coverts glutamate to 2-oxoglutarate (alpha-ketoglutarate). Glutamate-pyruvate transaminase 1 (GPT1) refers to any polypeptide (enzyme) that catalyzes reversible transamination between alanine and 2-oxoglutarate to form pyruvate and glutamate. Glutaminase (GLS) refers to any polypeptide (enzyme) that generates glutamate from glutamine. Amino acid sequences of exemplary PSAT1, GDH1, GPT1, and GLS are provided below:

GPT1
(SEQ ID NO: 89)
MASSTGDRSQAVRHGLRAKVLTLDGMNPRVRRVEYAVRGPIVQRALELEQELRQGVKKPFT
EVIRANIGDAQAMGQRPITFLRQVLALCVNPDLLSSPNFPDDAKKRAERILQACGGHSLGAYS
VSSGIQLIREDVARYIERRDGGIPADPNNVFLSTGASDAIVTVLKLLVAGEGHTRTGVLIPIPQY
PLYSATLAELGAVQVDYYLDEERAWALDVAELHRALGQARDHCRPRALCVINPGNPTGQV
QTRECIEAVIRFAFEERLFLLADEVYQDNVYAAGSQFHSFKKVLMEMGPPYAGQQELASFHS
TSKGYMGECGFRGGYVEVVNMDAAVQQQMLKLMSVRLCPPVPGQALLDLVVSPPAPTDPS
FAQFQAEKQAVLAELAAKAKLTEQVFNEAPGISCNPVQGAMYSFPRVQLPPRAVERAQELG
LAPDMFFCLRLLEETGICVVPGSGFGQREGTYHFRMTILPPLEKLRLLLEKLSRFHAKFTLEYS
GLS
(SEQ ID NO: 90)
MMRLRGSGMLRDLLLRSPAGVSATLRRAQPLVTLCRRPRGGGRPAAGPAAAARLHPWW
GGGGWPAEPLARGLSSSPSEILQELGKGSTHPQPGVSPPAAPAAPGPKDGPGETDAFGNSE
GKELVASGENKIKQGLLPSLEDLLFYTIAEGQEKIPVHKFITALKSTGLRTSDPRLKECMD
MLRLTLQTTSDGVMLDKDLFKKCVQSNIVLLTQAFRRKFVIPDFMSFTSHIDELYESAKKQ
SGGKVADYIPQLAKFSPDLWGVSVCTVDGQRHSTGDTKVPFCLQSCVKPLKYAIAVNDL
GTEYVHRYVGKEPSGLRFNKLFLNEDDKPHNPMVNAGAIVVTSLIKQGVNNAEKFDYVM
QFLNKMAGNEYVGFSNATFQSERESGDRNFAIGYYLKEKKCFPEGTDMVGILDFYFQLCS
IEVTCESASVMAATLANGGFCPITGERVLSPEAVRNTLSLMHSCGMYDFSGQFAFHVGLP
AKSGVAGGILLVVPNVMGMMCWSPPLDKMGNSVKGIHFCHDLVSLCNFHNYDNLRHFA
KKLDPRREGGDQRVKSVINLLFAAYTGDVSALRRFALSAMDMEQRDYDSRTALHVAAA
EGHVEVVKFLLEACKVNPFPKDRWNNTPMDEALHFGHHDVFKILQEYQVQYTPQGDSD
NGKENQTVHKNLDGLL
PSAT1
(SEQ ID NO: 91)
MDAPRQVVNFGPGPAKLPHSVLLEIQKELLDYKGVGISVLEMSHRSSDFAKIINNTENLVREL
LAVPDNYKVIFLQGGGCGQFSAVPLNLIGLKAGRCADYVVTGAWSAKAAEEAKKFGTINIV
HPKLGSYTKIPDPSTWNLNPDASYVYYCANETVHGVEFDFIPDVKGAVLVCDMSSNFLSKPV
DVSKFGVIFAGAQKNVGSAGVTVVIVRDDLLGFALRECPSVLEYKVQAGNSSLYNTPPCFSIY
VMGLVLEWIKNNGGAAAMEKLSSIKSQTIYEIIDNSQGFYVCPVEPQNRSKMNIPFRIGNAKG
DDALEKRFLDKALELNMLSLKGHRSVGGIRASLYNAVTIEDVQKLAAFMKKFLEMHQL
GDH1
(SEQ ID NO: 92)
MTYKCAVVDVPFGGAKAGVKINPKNYTDNELEKITRRFTMELAKKGFIGPGIDVPAPDMSTG
EREMSWIADTYASTIGHYDINAHACVTGKPISQGGIHGRISATGRGVFHGIENFINEASYMSIL
GMTPGFGDKTFVVQGFGNVGLHSMRYLHRFGAKCIAVGESDGSIWNPDGIDPKELEDFKLQ
HGSILGFPKAKPYEGSILEADCDILIPAASEKQLTKSNAPRVKAKIIAEGANGPTTPEADKIFLE
RNIMVIPDLYLNAGGVTVSYFEWLKNLNHVSYGRLTFKYERDSNYHLLMSVQESLERKFGK
HGGTIPIVPTAEFQDRISGASEKDIVHSGLAYTMERSARQIMRTAMKYNLGLDLRTAAYVNAI
EKVFKVYNEAGVTFT

The Krebs cycle modulating polypeptide may be a naturally-occurring polypeptide from a suitable species, for example, a mammalian Krebs cycle modulating polypeptide such as those derived from human or a non-human primate. Such naturally-occurring polypeptides are known in the art and can be obtained, for example, using any of the above-noted amino acid sequences as a query to search a publicly available gene database, for example GenBank. The Krebs cycle modulating polypeptide for use in the instant disclosure may share a sequence identity of at least 85% (e.g., 90%, 95%, 97%, 98%, 99%, or above) as any of the exemplary proteins noted above.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25 (17): 3389-3402, 1997. When utilizing BLAST and Gapped BLAST programs, the default parameters of the respective programs (e.g., XBLAST and NBLAST) can be used.

In some embodiments, the Krebs cycle modulating polypeptide may be conjugated to a subcellular compartment-localization signaling peptide (e.g., mitochondria-localization signaling peptide) for trafficking the polypeptide to a desired subcellular compartment. For example, a GOT2 polypeptide may comprise a mitochondria-localization signaling peptide such that it can be trafficked to mitochondria in a host immune cell.

Alternatively, the Krebs cycle modulating polypeptide may be a functional variant of a native counterpart. Such a functional variant may contain one or more mutations outside the functional domain(s) of the native counterpart. Functional domains of a native Krebs cycle modulating polypeptide may be known in the art or can be predicted based on its amino acid sequence. Mutations outside the functional domain(s) would not be expected to substantially affect the biological activity of the protein. In some instances, the functional variant may exhibit increased Krebs cycle modulation as relative to the native counterpart. Alternatively, the functional variant may exhibit decreased Krebs cycle modulation as relative to the native counterpart.

A functional variant of a Krebs cycle modulating polypeptide may be a functional variant of a wild-type polypeptide, which may comprise one or more mutations as relative to the native counterpart and retain substantially the same biological activity as the native counterpart. In some embodiments, the functional variant of the Krebs cycle modulating polypeptide comprises at least one, at least two, at least three, at least four, at least five, at least six, or more mutations as relative to the native counterpart.

For example, a functional variant of GOT may comprise at least one, at least two, at least three, at least four, at least five, at least six, or more mutations as relative to the native counterpart. In some embodiments, a functional variant of GOT comprises a mutation of a lysine residue at position 159 (e.g., K159Q) in SEQ ID NO: 88. In some embodiments, a functional variant of GOT comprises a mutation of a lysine residue at position 185 (e.g., K185Q) in SEQ ID NO: 88. In some embodiments, a functional variant of GOT comprises a mutation of a lysine residue at position 404 (e.g., K404Q) in SEQ ID NO: 88. In some embodiments, a functional variant of GOT comprises a mutation of a lysine residue at position 159 (e.g., K159Q) and position 185 (e.g., K185Q) in SEQ ID NO: 88. In some embodiments, a functional variant of GOT comprises a mutation of a lysine residue at position 185 (e.g., K185Q) and position 404 (e.g., K404Q) in SEQ ID NO: 88. In some embodiments, a functional variant of GOT comprises a mutation of a lysine residue at position 159 (e.g., K159Q) and position 404 (e.g., K404Q) in SEQ ID NO: 88. In some embodiments, a functional variant of GOT comprises a mutation of a lysine residue at position 159 (e.g., K159Q), position 185 (e.g., K185Q), and position 404 (e.g., K404Q) in SEQ ID NO: 88. See also Yang et al., The EMBO Journal (2015) 34:1100-1125, the relevant disclosures of which are incorporated by reference herein for the purpose and subject matter reference herein.

In some embodiments, the functional variant of the Krebs cycle modulating polypeptide may exhibit one or more biological properties (e.g., modification status, catalytic activity, cellular location and/or binding partners) that may be altered as relative to the native counterpart. Non-limiting examples of a functional variant of a Krebs cycle modulating polypeptide include a functional variant of an enzyme that catalyzes a reaction in the Krebs cycle (e.g., a functional variant of IDH, MDH, or PHGDH), a functional variant of an enzyme that uses a Krebs cycle metabolite as a substrate (e.g., a functional variant of GOT or PCK1), and a functional variant of an enzyme that converts a precursor to a Krebs cycle metabolite (e.g., a functional variant of PSAT1, GDH1, GPT1, or GLS).

Alternatively or in addition, the functional variant may contain a conservative mutation(s) at one or more positions in the native counterpart (e.g., up to 20 positions, up to 15 positions, up to 10 positions, up to 5, 4, 3, 2, 1 position(s)). As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (c) S, T; (f) Q, N; and (g) E, D.

### II. Chimeric Receptor Polypeptides

As used herein, a chimeric receptor polypeptide refers to a non-naturally occurring molecule that can be expressed on the surface of a host cell. A chimeric receptor polypeptide comprises an extracellular target binding domain that can target an antigen of interest (e.g., an antigen associated with a disease such as cancer or an antigen associated with a pathogen; see discussions herein). An extracellular target binding domain may bind to an antigen of interest directly (e.g., an extracellular antigen binding domain in a CAR polypeptide as disclosed herein). Alternatively, an extracellular target binding domain may bind to the antigen of interest via an intermediate, for example, an Fc-containing agent such as an antibody. A chimeric receptor polypeptide may further comprise a transmembrane domain, a hinge domain, a cytoplasmic signaling domain, one or more co-stimulatory domains, a cytoplasmic signaling domain, or a combination thereof. In some instances, the chimeric receptor polypeptide may be free of co-stimulatory domains. The chimeric receptor polypeptides are configured such that, when expressed on a host cell, the extracellular target binding domain is located extracellularly for binding to a target antigen, directly or indirectly. The optional co-stimulatory signaling domain may be located in the cytoplasm for triggering activation and/or effector signaling.

In some embodiments, chimeric receptor polypeptides described herein may further comprise a hinge domain, which may be located at the C-terminus of the extracellular target binding domain and the N-terminus of the transmembrane domain. The hinge may be of any suitable length. In other embodiments, the chimeric receptor polypeptide described herein may have no hinge domain at all. In yet other embodiments, the chimeric receptor polypeptide described herein may have a shortened hinge domain (e.g., including up to 25 amino acid residues).

In some embodiments, a chimeric receptor polypeptide as described herein may comprise, from N-terminus to C-terminus, the extracellular target binding domain, the transmembrane domain, and the cytoplasmic signaling domain. In some embodiments, a chimeric receptor polypeptide as described herein comprises, from N-terminus to C-terminus, the extracellular target binding domain, the transmembrane domain, at least one co-stimulatory signaling domain, and the cytoplasmic signaling domain. In other embodiments, a chimeric receptor polypeptide as described herein comprises, from N-terminus to C-terminus, the extracellular target binding domain, the transmembrane domain, the cytoplasmic signaling domains, and at least one co-stimulatory signaling domain.

In some embodiments, the chimeric receptor polypeptide can be an antibody-coupled T cell receptor (ACTR) polypeptide. As used herein, an ACTR polypeptide (a.k.a., an ACTR construct) refers to a non-naturally occurring molecule that can be expressed on the surface of a host cell and comprises an extracellular domain with binding affinity and specificity for the Fc portion of an immunoglobulin (“Fc binder” or “Fc binding domain”), a transmembrane domain, and a cytoplasmic signaling domain. In some embodiments, the ACTR polypeptides described herein may further include at least one co-stimulatory signaling domain.

In other embodiments, the chimeric receptor polypeptide disclosed herein may be a chimeric antigen receptor (CAR) polypeptide. As used herein, a CAR polypeptide (a.k.a., a CAR construct) refers to a non-naturally occurring molecule that can be expressed on the surface of a host cell and comprises an extracellular antigen binding domain, a transmembrane domain, and a cytoplasmic signaling domain. The CAR polypeptides described herein may further include at least one co-stimulatory signaling domain.

The extracellular antigen binding domain may be any peptide or polypeptide that specifically binds to a target antigen, including naturally occurring antigens that are associated with a medical condition (e.g., a disease), or an antigenic moiety conjugated to a therapeutic agent that targets a disease-associated antigen.

In some embodiments, the CAR polypeptides described herein may further include at least one co-stimulatory signaling domain. The CAR polypeptides are configured such that, when expressed on a host cell, the extracellular antigen-binding domain is located extracellularly for binding to a target molecule and the cytoplasmic signaling domain. The optional co-stimulatory signaling domain may be located in the cytoplasm for triggering activation and/or effector signaling.

As used herein, the phrase “a protein X transmembrane domain” (e.g., a CD8 transmembrane domain) refers to any portion of a given protein, i.e., transmembrane-spanning protein X, that is thermodynamically stable in a membrane.

As used herein, the phrase “a protein X cytoplasmic signaling domain,” for example, a CD3ζ cytoplasmic signaling domain, refers to any portion of a protein (protein X) that interacts with the interior of a cell or organelle and is capable of relaying a primary signal as known in the art, which lead to immune cell proliferation and/or activation. The cytoplasmic signaling domain as described herein differs from a co-stimulatory signaling domain, which relays a secondary signal for fully activating immune cells.

As used herein, the phrase “a protein X co-stimulatory signaling domain,” e.g., a CD28 co-stimulatory signaling domain, refers to the portion of a given co-stimulatory protein (protein X, such as CD28, 4-1BB, OX40, CD27, or ICOS) that can transduce co-stimulatory signals (secondary signals) into immune cells (such as T cells), leading to fully activation of the immune cells.

### A. Extracellular Target Binding Domain

The chimeric receptor polypeptides disclosed herein comprise an extracellular domain that targets an antigen of interest (e.g., those described herein) via either direct binding or indirectly binding (through an intermediate such as an antibody). The chimeric receptor polypeptides may be ACTR polypeptides that comprise an Fc binding domain. Alternatively, the chimeric receptor polypeptides may be CAR polypeptides that comprise an extracellular antigen binding domain.

### Fc Binding Domains

The ACTR polypeptides described herein comprise an extracellular domain that is an Fc binding domain, i.e., capable of binding to the Fc portion of an immunoglobulin (e.g., IgG, IgA, IgM, or IgE) of a suitable mammal (e.g., human, mouse, rat, goat, sheep, or monkey). Suitable Fc binding domains may be derived from naturally occurring proteins such as mammalian Fc receptors or certain bacterial proteins (e.g., protein A, protein G). Additionally, Fc binding domains may be synthetic polypeptides engineered specifically to bind the Fc portion of any of the antibodies described herein with high affinity and specificity. For example, such an Fc binding domain can be an antibody or an antigen-binding fragment thereof that specifically binds the Fc portion of an immunoglobulin. Examples include, but are not limited to, a single-chain variable fragment (scFv), a domain antibody, or single domain antibodies (e.g., nanobodies). Alternatively, an Fc binding domain can be a synthetic peptide that specifically binds the Fc portion, such as a Kunitz domain, a small modular immunopharmaceutical (SMIP), an adnectin, an avimer, an affibody, a DARPin, or an anticalin, which may be identified by screening a peptide combinatory library for binding activities to Fc.

In some embodiments, the Fc binding domain is an extracellular ligand-binding domain of a mammalian Fc receptor. As used herein, an “Fc receptor” is a cell surface bound receptor that is expressed on the surface of many immune cells (including B cells, dendritic cells, natural killer (NK) cells, macrophage, neutrophils, mast cells, and eosinophils) and exhibits binding specificity to the Fc domain of an antibody. Fc receptors are typically comprised of at least two immunoglobulin (Ig)-like domains with binding specificity to an Fc (fragment crystallizable) portion of an antibody. In some instances, binding of an Fc receptor to an Fc portion of the antibody may trigger antibody dependent cell-mediated cytotoxicity (ADCC) effects. The Fc receptor used for constructing an ACTR polypeptide as described herein may be a naturally-occurring polymorphism variant (e.g., the CD16 V158 variant), which may have increased or decreased affinity to Fc as compared to a wild-type counterpart. Alternatively, the Fc receptor may be a functional variant of a wild-type counterpart, which carry one or more mutations (e.g., up to 10 amino acid residue substitutions including 1, 2, 3, 4, 5, 6, 7, 8, 9, or 10 mutations) that alter the binding affinity to the Fc portion of an Ig molecule. In some instances, the mutation may alter the glycosylation pattern of the Fc receptor and thus the binding affinity to Fc.

The table below lists a number of exemplary polymorphisms in Fc receptor extracellular domains (see, e.g., Kim et al., J. Mol. Evol. 53:1-9, 2001) which may be used in any of the methods or constructs described herein:

TABLE 1
Exemplary Polymorphisms in Fc Receptors

| Amino | | | | | | | | | | |
|---|---|---|---|---|---|---|---|---|---|---|
| Acid | | | | | | | | | | |
| Number | 19 | 48 | 65 | 89 | 105 | 130 | 134 | 141 | 142 | 158 |
| FCR10 | R | S | D | I | D | G | F | Y | T | V |
| P08637 | R | S | D | I | D | G | F | Y | I | F |
| S76824 | R | S | D | I | D | G | F | Y | I | V |
| J04162 | R | N | D | V | D | D | F | H | I | V |
| M31936 | S | S | N | I | D | D | F | H | I | V |
| M24854 | S | S | N | I | E | D | S | H | I | V |
| X07934 | R | S | N | I | D | D | F | H | I | V |
| X14356 | N | N | N | S | E | S | S | S | I | I |
| (FcγRII) | | | | | | | | | | |
| M31932 | S | T | N | R | E | A | F | T | I | G |
| (FcγRI) | | | | | | | | | | |
| X06948 | R | S | E | S | Q | S | E | S | I | V |
| (FcαεI) | | | | | | | | | | |

Fc receptors are classified based on the isotype of the antibody to which it is able to bind. For example, Fc-gamma receptors (FcγR) generally bind to IgG antibodies, such as one or more subtype thereof (i.e., IgG1, IgG2, IgG3, IgG4); Fc-alpha receptors (FcαR) generally bind to IgA antibodies; and Fc-epsilon receptors (FcεR) generally bind to IgE antibodies. In some embodiments, the Fc receptor is an Fc-gamma receptor, an Fc-alpha receptor, or an Fc-epsilon receptor. Examples of Fc-gamma receptors include, without limitation, CD64A, CD64B, CD64C, CD32A, CD32B, CD16A, and CD16B. An example of an Fc-alpha receptor is FcαR1/CD89. Examples of Fc-epsilon receptors include, without limitation, FcεRI and FcεRII/CD23. The table below lists exemplary Fc receptors for use in constructing the ACTR polypeptides described herein and their binding activity to corresponding Fc domains:

TABLE 2
Exemplary Fc Receptors

| Receptor name | Principal antibody ligand | Affinity for ligand |
|---|---|---|
| FcγRI (CD64) | IgG1 and IgG3 | High (Kd ~ 10⁻⁹M) |
| FcγRIIA (CD32) | IgG | Low (Kd > 10⁻⁷M) |
| FcγRIIB1 (CD32) | IgG | Low (Kd > 10⁻⁷M) |
| FcγRIIB2 (CD32) | IgG | Low (Kd > 10⁻⁷M) |
| FcγRIIIA (CD16a) | IgG | Low (Kd > 10⁻⁶M) |
| FcγRIIIB (CD16b) | IgG | Low (Kd > 10⁻⁶M) |
| FcεRI | IgE | High (Kd ~ 10⁻¹⁰M) |
| FcεRII (CD23) | IgE | Low (Kd > 10⁻⁷M) |
| FcαRI (CD89) | IgA | Low (Kd > 10⁻⁶M) |
| Fcα/μR | IgA and IgM | High for IgM, |
| | | Mid for IgA |
| FcRn | IgG | |

Selection of the ligand binding domain of an Fc receptor for use in the ACTR polypeptides described herein will be apparent to one of skill in the art. For example, it may depend on factors such as the isotype of the antibody to which binding of the Fc receptor is desired and the desired affinity of the binding interaction.

The extracellular antigen binding domain of any of the CAR polypeptides In some examples, the Fc binding domain is the extracellular ligand-binding domain of CD16, which may incorporate a naturally occurring polymorphism that may modulate affinity for Fc. In some examples, the Fc binding domain is the extracellular ligand-binding domain of CD16 incorporating a polymorphism at position 158 (e.g., valine or phenylalanine). In some embodiments, the Fc binding domain is produced under conditions that alter its glycosylation state and its affinity for Fc.

The amino acid sequences of human CD16A F158 and CD16A V158 variants are provided below with the F158 and V158 residue highlighted in bold/face and underlined (signal peptide italicized):

CD16A F158 (SEQ ID NO: 93):
MWQLLLPTALLLLVSAGMRTEDLPKAVVFLEPQWYRVLEKDSVTLKCQGAYSPEDNS
TQWFHNESLISSQASSYFIDAATVDDSGEYRCQTNLSTLSDPVQLEVHIGWLLLQAPR
WVFKEEDPIHLRCHSWKNTALHKVTYLQNGKGRKYFHHNSDFYIPKATLKDSGSYF
CRGLFGSKNVSSETVNITITQGLAVSTISSFFPPGYQVSFCLVMVLLFAVDTGLYFSVK
TNIRSSTRDWKDHKFKWRKDPQDK
CD16A V158 (SEQ ID NO: 94):
MWQLLLPTALLLLVSAGMRTEDLPKAVVFLEPQWYRVLEKDSVTLKCQGAYSPED
NSTQWFHNESLISSQASSYFIDAATVDDSGEYRCQTNLSTLSDPVQLEVHIGWLLL
QAPRWVFKEEDPIHLRCHSWKNTALHKVTYLQNGKGRKYFHHNSDFYIPKATLK
DSGSYFCRGLVGSKNVSSETVNITITQGLAVSTISSFFPPGYQVSFCLVMVLLFAVD
TGLYFSVKTNIRSSTRDWKDHKFKWRKDPQDK

In some embodiments, the Fc binding domain is the extracellular ligand-binding domain of CD16 incorporating modifications that render the ACTR polypeptide specific for a subset of IgG antibodies. For example, mutations that increase or decrease the affinity for an IgG subtype (e.g., IgG1) may be incorporated.

Any of the Fc binding domains described herein may have a suitable binding affinity for the Fc portion of a therapeutic antibody. As used herein, “binding affinity” refers to the apparent association constant or K_{A}. The K_{A} is the reciprocal of the dissociation constant, K_{D}. The extracellular ligand-binding domain of an Fc receptor domain of the ACTR polypeptides described herein may have a binding affinity K_{d} of at least 10⁻⁵, 10⁻⁶, 10⁻⁷, 10⁻⁸, 10⁻⁹, 10⁻¹⁰ M or lower for the Fc portion of antibody. In some embodiments, the Fc binding domain has a high binding affinity for an antibody, isotype(s) of antibodies, or subtype(s) thereof, as compared to the binding affinity of the Fc binding domain to another antibody, isotype(s) of antibodies, or subtypes(s) thereof. In some embodiments, the extracellular ligand-binding domain of an Fc receptor has specificity for an antibody, isotype(s) of antibodies, or subtype(s) thereof, as compared to binding of the extracellular ligand-binding domain of an Fc receptor to another antibody, isotype(s) of antibodies, or subtypes(s) thereof.

Other Fc binding domains as known in the art may also be used in the ACTR constructs described herein including, for example, those described in WO2015058018A1 and PCT Application No.: PCT/US2018/015999, the relevant disclosures of each of which are incorporated by reference for the purpose and subject matter referenced herein.

### Extracellular Antigen Binding Domains

The CAR polypeptides described herein comprise an extracellular antigen binding domain, which re-directs the specificity of immune cells expressing the CAR polypeptide. As used herein, “an extracellular antigen binding domain” refers to a peptide or polypeptide having binding specificity to a target antigen of interest, which can be a naturally occurring antigen associated with a medical condition (e.g., a disease), or an antigenic moiety conjugated to a therapeutic agent that targets a disease-associated antigen. The extracellular antigen binding domain as described herein does not comprise an extracellular domain of an Fc receptor, and may not bind to the Fc portion of an immunoglobulin. An extracellular domain that does not bind to an Fc fragment means that the binding activity between the two is not detectable using a conventional assay or only background or biologically insignificant binding activity is detected using the conventional assay.

In some instances, the extracellular antigen binding domain of any CAR polypeptides described herein is a peptide or polypeptide capable of binding to a cell surface antigen (e.g., a tumor antigen), or an antigen (or a fragment thereof) that is complex with a major histocompatibility complex and be presented on the cell surface of an antigen-presenting cell. Such an extracellular antigen binding domain may be a single-chain antibody fragment (scFv), which may be derived from an antibody that binds the target cell surface antigen with a high binding affinity. Table 1 below lists exemplary cell-surface target antigens and exemplary antibodies binding to such.

TABLE 3
Exemplary Cell Surface Target Antigen and Exemplary Antibodies Binding to Such

| Exemplary | | Exemplary | Exemplary |
|---|---|---|---|
| Target | Exemplary | Target | Antibodies and |
| Antigens | Antibodies | Antigens | Fc-fusion Agents |
| CD137 (4-1BB) | utomilumab | CD74 | milatuzumab |
| Trophoblast | naptumomab | HLA-DR | IMMU-114 |
| glycoprotein (5T4) | estafenatox | | |
| Adenosine A2a | anti-A2aR | Hsp70 | mi-TUMEXtx |
| receptor (A2aR) | mAbs | | |
| Alk-1 protein kinase | ascrinvacumab | Hsp 90 | ZSG-102 |
| (ACVRL1) | | | |
| ADAM-10 | 8C7 | ICAM-1 | BI-505 |
| (ADAM10) | | | |
| TACE | MEDI-3622 | Inducible T-cell co- | GSK-3359609 |
| (ADAM17) | | stimulator (ICOS) | |
| ADAM-28 | GFC-201 | Immunoglobulin | KappaMab |
| (ADAM28) | | kappa (Ig kappa) | |
| CD156; | MAB-1031 | Immunoglobulin | LambdaMab |
| Immunoglobulin G1; | | antigen (Ig lambda) | |
| Immunoglobulin G2 | | | |
| (ADAM8) | | | |
| ADAM-9 | AEX-6003 | IL-6 receptor | tocilizumab |
| (ADAM9) | | (IL-6R) | |
| Anterior gradient | agtuzumab | IL-7 receptor | anti-IL7R |
| protein 2 homolog | | (IL-7R) | mAbs |
| (AGR2) | | | |
| Anaplastic lymphoma | KTN-0125 | IL-13 receptor alpha 1 | ASLAN-004 |
| kinase (ALK) | | subunit (IL13RA1) | |
| Angiopoietin ligand- | vanucizumab | IL-13 receptor alpha 2 | anti-IL13RA2 |
| 2 (Ang-2); Vascular | | subunit (IL13RA2) | mAbs |
| endothelial growth | | | |
| factor-A (VEGF-A) | | | |
| Lactadherin (Anti- | TriAb (11D10) | IL-1 receptor | CAN-04 |
| idiotype) | | accessory protein | |
| | | (IL1RAP) | |
| Tumor necrosis | BION-1301 | IL-2 receptor beta | Mikbeta1 |
| factor ligand 13 | | (IL2R beta) | |
| (APRIL) | | | |
| Aspartate beta- | PAN-622 | Immunoglobulin like | BAY-1905254 |
| hydroxylase (ASPH) | | domain receptor 2 | |
| | | (ILDR2) | |
| Axl tyrosine kinase | BA-3011 | Integrin alpha-X/beta- | anti-Integrin a10b1 |
| (AXL) | | 1 (Integrin a10b1) | mAbs |
| CD276 antigen | BVD m276; | Integrin alpha-3/beta- | BCMab-1 |
| (B7-H3) | hu8H9 | 1 (Integrin a3b1) | |
| V-set domain- | FPA-150 | Integrin alpha-6/beta- | 90Y-ITGA6B4 |
| containing T-cell | | 4 (Integrin a6b4) | |
| activation inhibitor 1 | | | |
| (VTCN1; also | | | |
| B7-H4) | | | |
| B-cell activating | blisibimod | Integrin alpha-9 | GND-001 |
| factor; (BAFF; also | | (Integrin a9) | |
| TNFSF13B and | | | |
| CD257) | | | |
| B-cell activating | VAY736 | CD49b (Integrin | Vatelizumab |
| factor receptor; | | alpha 2) | |
| (BAFF-R; also | | | |
| TNFSF13C and | | | |
| CD268) | | | |
| BAG molecular | anti-BAG3 | CD49c (Integrin | anti-CD49c |
| chaperone regulator 3 | mAbs | alpha 3) | mAbs |
| (BAG3) | | | |
| Basigin (BSG; | cHAb18 | CD49d; (Integrin | anti-CD49d |
| CD147) | | alpha 4) | mAbs |
| B-cell maturation | SEA-BCMA | CD51 | abituzumab |
| antigen (BCMA; also | | | |
| TNFRSF17) | | | |
| ADP ribosyl cyclase- | OX-001 | CD29 (integrin | OS-2966 |
| 2 (BST1) | | beta 1) | |
| B and T lymphocyte | 40E4 | CD61 (Integrin | anti-CD61 |
| attenuator (BTLA) | | beta 3) | mAbs |
| Complement C5a | neutrazumab | Jagged-1 | anti-Jagged-1 |
| receptor (C5aR) | | | mAbs |
| CACNA2D1 calcium | anti-CACNA2D1 | Kidney-associated | AB-3A4 |
| channel subunit | mAbs | antigen 1 (KAAG1) | |
| (CACNA2D1) | | | |
| Carbonic anhydrase- | G250 | Potassium channel | Y-4 |
| IX (CAIX) | | subfamily K member | |
| | | 9 (KCNK9) | |
| Calreticulin (CALR) | Anti-CALR mAbs | KIR2DL1/2L3 | lirilumab |
| Caveolin 1 (CAV1) | anti-CAV1 mAbs | tyrosine-protein | CDX-0158 |
| | | kinase kit (KIT) | |
| Carbonic anhydrase- | 177Lu-6A10-Fab; | L1CAM | anti-L1CAM |
| XII (CAXII) | anti-CAXII mAbs | | mAbs |
| CCR2 chemokine | plozalizumab | Death receptor 5 | APOMAB |
| receptor (CCR2) | | (DR5) | |
| CCR3 chemokine | anti-CCR3 | CD223 (LAG3) | relatlimab |
| receptor (CCR3) | mAbs | | |
| CCR4 chemokine | mogamulizumab | Lewis Y | hu3S193; MB311 |
| receptor (CCR4) | | | |
| CCR5 chemokine | PRO 140; | Zinc transporter | SGN-LIV1 |
| receptor (CCR5) | CCR5mAb004 | SLC39A6 (LIV1) | |
| CCR7 chemokine | anti-CCR7 | Lysyl oxidase-like | AB-0023 |
| receptor (CCR7) | mAbs | protein 2 (LOXL2) | |
| CCR9 chemokine | anti-CCR9 | Leucine rich repeat- | ABBV-085 |
| receptor (CCR9) | mAbs | containing protein 15 | |
| | | (LRRC15) | |
| Interleukin-3 | CSL362; | Leucine rich repeat- | ARGX-115 |
| receptor alpha | KHK2823 | containing protein 32 | |
| (IL3RA; CD123) | | (LRRC32) | |
| Aminopeptidase N | MI-130110 | Lymphocyte antigen | MEN-1309 |
| (CD13) | | 75 (LY75) | |
| Prominin 1 (CD133) | anti-CD133 | Ly6/PLAUR domain- | BAY-1129980 |
| | mAbs | containing protein 3 | |
| | | (LYPD3) | |
| Syndecan-1 (CD138) | indatuximab | Melanoma associated | LxC-002 |
| | ravtansine | antigen (MAGE | |
| | | peptide presented | |
| | | in MHC) | |
| CD160 | ELB-021 | Matriptase (ST14) | anti-ST14 mAbs |
| Activated leukocyte | CX-2009 | MICA/B | IPH4301 |
| cell adhesion | | | |
| molecule (CD166) | | | |
| B-lymphocyte | MOR208 | MIF/HLA-A2 (MIF | RL21A |
| antigen CD19 | | peptide presented | |
| | | in MHC) | |
| B-lymphocyte | rituximab; | Anti-mullerian | GM-102 |
| antigen CD20 | obinituzumab; | hormone II (MHR2) | |
| | ocaratuzumab | | |
| Membrane | samalizumab | MMP1/HLA | Anti-MMP1/HLA |
| glycoprotein OX2 | | (MMP1 peptide | mAbs |
| CD200 | | presented in MHC1) | |
| CD22 | epratuzumab | Metalloprotease-9 | andecaliximab |
| | | (MMP9) | |
| Immunoglobulin | lumiliximab | Mesothelin (MSLN) | MORAb-009 |
| epsilon Fc receptor II | | | |
| (CD23) | | | |
| Signal transducer | anti-CD24 mAbs | Mucin 1 (MUC1) | PankoMab-GEX |
| CD24 | | | |
| IL-2 receptor alpha | 90Y-daclizumab | Mucin 13 (MUC13) | anti-MUC13 mAbs |
| subunit CD25 | | | |
| CD27 | varilumab | Endomucin (MUC14) | anti-MUC14 mAbs |
| CD28 | theralizumab | Mucin 16 (MUC16) | sofituzumab |
| CD3 | Muromonab-CD3 | Cell surface | AA98 |
| | (OKT3) | glycoprotein MUC18 | |
| | | (CD146) | |
| CD30 | brentuximab | Mucin 5AC | ensituximab |
| | vedotin | (MUC5AC) | |
| Immunoglobulin | BI-1206 | N-glycolyl GM3 | 99mTc-labeled |
| gamma Fc receptor | | (NeuGcGM3) | 14F7 |
| IIB (CD32B) | | | |
| CD33 | lintuzumab | Sodium-dependent | XMT-1536 |
| | | phosphate transport | |
| | | protein 2B | |
| | | (SLC34A2) | |
| CD37 | otlertuzumab | Nucleolin (NCL) | anti-nucleolin |
| | | | mAbs |
| ADP ribosyl cyclase- | daratumumab | Nectin-4 | enfortumab vedotin |
| 1 (CD38) | | | |
| CD39 | OREG-103 | Neurofibromin (NF1) | anti-neurofibromin |
| | | | mAbs |
| CD4 | IT-1208 | NGcGM3 ganglioside | racotumomab |
| CD40 | lucatumumab | NKG2A | monalizumab |
| CD43 | leukotuximab | non-POU domain- | PAT-LM1 |
| | | containing octamer- | |
| | | binding protein | |
| | | (NONO) | |
| CD44 | RG7356 | Notch-1 | brontictuzumab |
| CD45 | 131I-BC8 | CD73 | oleclumab |
| Membrane cofactor | AugmAb | Netrin-1 (NTN1) | NP-137 |
| protein (CD46) | | | |
| CD47 | Hu5F9-G4 | OX-40 | PF-04518600 |
| CD52 | alemtuzumab | P2X purinoceptor 7 | BIL-010t |
| | | (P2RX7) | |
| CD55 | PAT-SC1 | FGF receptor | MM-161 |
| | | (pan FGFR) | |
| Neural cell adhesion | IMGN-901 | Integrin | NOD201 |
| molecule 1; (CD56) | | (Pan integrin) | |
| T-cell | itolizumab | P-cadherin, | PCA-062 |
| differentiation | | also cadherin-3 | |
| antigen CD6 | | (CDH3) | |
| CD70 | SGN-70 | Programmed cell | pembrolizumab |
| | | death protein 1 (PD-1) | |
| CD79b | polatuzumab | Programmed cell | avelumab; Euchloe |
| | vedotin | death ligand 1 | H12 |
| | | (PD-L1) | |
| CD8 | anti-CD8 | Programmed cell | rHIgM12B7 |
| | mAbs | death ligand 2 | |
| | | (PD-L2) | |
| CD80 | galiximab | PDGF receptor | olaratumumab |
| | | alpha (PDGFRA) | |
| CD98 | IGN-523 | Placenta specific | anti-PLAC1 |
| | | protein 1 (PLAC1) | mAbs |
| CD99 | NV-103 | PR1/HLA (PR1 | anti-PR1/HLA |
| | | peptide in MHC) | mAbs |
| Cadherin-1 | anti-CDH1 | Prolactin receptor | ABBV-176 |
| (CDH1) | mAbs | PRLR | |
| Cadherin-17 | anti-CDH17 | Phosphatidylserine | anti- |
| (CDH17) | mAbs | | phosphatidylserine |
| | | | mAbs |
| Cadherin 19 | anti-CDH19 | Prostate stem cell | anti-PSCA |
| (CDH19) | mAbs | antigen (PSCA) | mAbs |
| Cadherin-6 | HKT-288 | Glutamate | ATL-101 |
| (CDH6) | | carboxypeptidase II | |
| | | (PSMA) | |
| CD66a | CM-24 | Parathyroid hormone- | CAL |
| (CEACAM1) | | related protein | |
| | | (PTH-rP) | |
| CD66e | IMMU-130 | Tyrosine-protein | cofetuzumab |
| (CEACAM5) | | kinase-like 7 (PTK7) | pelidotin |
| CD66c; CD66e | NEO-201 | Protein tyrosine | PRL3-zumab |
| (CEACAM5/6) | | phosphatase IVA3 | |
| | | (PTP4A3) | |
| Claudin 18 | IMAB362 | Poliovirus receptor | COM-701 |
| (Claudin 18.2) | | related | |
| | | immunoglobulin | |
| | | domain containing | |
| | | (PVRIG) | |
| Claudin 6 | IMAB027 | Receptor activator of | denosumab |
| | | nuclear factor kappa- | |
| | | B ligand (RANKL) | |
| SLAM family | elotuzumab | Recepteur d'origine | anti-RON mAbs |
| member 7 (CS1) | | nantais | |
| | | (RON) | |
| colony stimulating | cabiralizumab | Tyrosine-protein | cirmtuzumab |
| factor-1 receptor | | kinase transmembrane | |
| (CSF1R) | | receptor ROR1 | |
| | | (ROR1); also | |
| | | NTRKR1 | |
| Cytotoxic T- | ipilumumab | Tyrosine-protein | BA-3021 |
| lymphocyte | | kinase transmembrane | |
| protein-4 | | receptor ROR2 | |
| (CTLA4) | | (ROR2); also | |
| | | NTRKR2 | |
| Coxsackievirus and | anti-CXADR | R-spondin-3 (RSPO3) | rosmantuzumab |
| adenovirus receptor | mAbs | | |
| (CXADR) | | | |
| CXCR2 chemokine | anti-CXCR2 | Sphingosine-1- | EDD7H9 |
| receptor | mAbs | phosphate receptor 3 | |
| | | (S1PR3) | |
| CXCR3 chemokine | anti-CXCR3 | Surface Antigen In | IGN-786 |
| receptor | mAbs | Leukemia (SAIL) | |
| CXCR4 chemokine | ulocuplumab | Semaphorin-4D | VX-15 |
| receptor | | (SEMA4D) | |
| CXCR5 chemokine | STI-B030X | carbohydrate antigen | MVT-1075 |
| receptor | | 19-9 (CA 19-9) | |
| CXCR7 chemokine | anti-CXCR7 | Sialyl Thomsen | anti-STn |
| receptor | mAbs | nouveau antigen | mAbs |
| | | (STn) | |
| DCLK1 | anti-DCLK1 | Sialic acid-binding Ig- | AK-002 |
| | mAbs | like lectin 8 (Siglec-8) | |
| Dickkopf-related | BHQ-880 | Sialic acid-binding Ig- | anti-Siglec-9 |
| protein 1 (DKK1) | | like lectin 9 (Siglec-9) | mAbs |
| DLK1 | ADCT-701 | Signal Regulatory | OSE-172 |
| | | Protein Alpha | |
| | | (SIRPA) | |
| Delta-like protein | SC16LD6.5 | CD48; also SLAM | SGN-CD48A |
| ligand 3 (DLL3) | | family member 2 | |
| | | (SLAMF2) | |
| Delta-like protein | navicixizumab | CD352; SLAM family | SGN-CD352A |
| ligand 4 (DLL4); | | member 6 (SLAMF6) | |
| VEGF (VEGF) | | | |
| Dipeptidyl peptidase- | YSCMA | Neutral amino acid | KM-8094 |
| 4 (DPP4), (also | | transporter B0 | |
| CD26) | | (SLC1A5) | |
| Death receptor-3 | PTX-35 | Somatostatin 2 | XmAb-18087 |
| (DR3) | | receptor (SSTR2) | |
| TRAIL-1 receptor | HuYON007 | Stabilin 1 (STAB1) | FP-1305 |
| (DR4) | MultYbody | | |
| TRAIL-1 receptor; | DR4/DR5 | Metalloreductase | 89Zr-DFO- |
| TRAIL-2 receptor | Surrobody | (STEAP1) | MSTP2109A |
| (DR4/DR5) | | | |
| TRAIL-2 receptor | DS-8273 | Survivin | anti-survivin |
| (DR5) | | | mAbs |
| EGF-like protein 6 | anti-EGFL6 | TAG-72 | 90Y-IDEC-159 |
| (EGFL6) | mAbs | | |
| Epidermal growth | cetuximab; | T cell | anti-TCR |
| factor receptor | Sym004; | receptor (TCR) | mAbs |
| (EGFR) | nimotuzumab | | |
| Epidermal growth | ABT-806 | Endosialin (TEM1) | ontuxizumab |
| factor receptor vIII | | | |
| (EGFRvIII) | | | |
| Epithelial membrane | ONCR-201 | Anthrax toxin | anti-TEM8 |
| protein 2 (EMP2) | | receptor 1 | mAbs |
| | | (ANTXR1); also | |
| | | TEM8 | |
| Endoglin | carotuximab | Tissue factor (TF) | MORAb-066 |
| Ectonucleotide | AGS-16C3F | Transforming growth | anti-TGFBR2 |
| pyrophosphatase/ | | factor, beta receptor II | mAbs |
| phosphodiesterase | | TGF-beta type II | |
| family member 3 | | (TGFBR2) | |
| (ENPP3) | | | |
| Prostaglandin | anti-PTGER2 | Thomsen- | JAA-F11 |
| E₂ receptor 2 | mAbs | Friedenreich | |
| (PTGER2) | | Antigen | |
| Prostaglandin | anti-PTGER4 | T cell | BMS-986207 |
| E₂ receptor 4 | mAbs | immunoreceptor with | |
| (PTGER4) | | Ig and ITIM domains | |
| | | (TIGIT) | |
| EpCAM | oportuzumab | Hepatitis A virus | CDX-014 |
| | monatox | cellular receptor 1 | |
| | | (HAVCR1); also | |
| | | TIM-1 | |
| Ephrin type-A | MEDI-547 | Hepatitis A virus | MBG453 |
| receptor 2 | | cellular receptor 2 | |
| (EphA2) | | (HAVCR2); also | |
| | | TIM-3 | |
| Ephrin type-A | KB004 | Toll-like receptor 2 | OPN-305 |
| receptor 3 (EphA3) | | (TLR-2) | |
| Fibroblast activation | F19 | Toll-like receptor 4 | anti-TLR4 |
| protein (FAP) | | (TLR-4) | mAbs |
| CD95 (FAS) | asunercept | Transmembrane 4 L6 | anti-TM4SF1 |
| | | family member 1 | mAbs |
| | | (TM4SF1) | |
| Fc receptor like | RG-6160 | Tumor necrosis factor | anti-TNFR2 |
| protein 5 (FCRL5) | | receptor 2 (TNFR2) | mAbs |
| FGF receptor 1 | FP-1039 | CD71 | anti-CD71 |
| (FGFR1) | | | mAbs |
| FGF receptor 2b | FPA-144 | Triggering receptor | anti-TREM1 |
| (FGFR2b) | | expressed on myeloid | mAbs |
| | | cells 1 (TREM1) | |
| FGF receptor 3 | B-701 | Tumor-associated | DS-1062 |
| (FGFR3) | | calcium signal | |
| | | transducer 2 (Trop-2) | |
| fms-like tyrosine | Flysyn | TWEAK Receptor | MRT-101 |
| kinase 3 (FLT3) | | (TWEAKR) | |
| Folate receptor | farletuzumab; | Tyrosine-protein | ELB-031 |
| alpha | IMGN853; | kinase receptor | |
| (FOLR1) | KHK2805 | TYRO3 (TYRO3) | |
| Folate receptor | anti-FOLR | Urokinase | MNPR-101 |
| beta | beta mAbs | receptor | |
| (FOLR2) | | (uPAR) | |
| Frizzled-1; Frizzled- | vantictumab | VEGF-2 | ramucirumab |
| 2; Frizzled-5; | | (VEGFR2) | |
| Frizzled-7; Frizzled- | | | |
| 8; (FZD1, 2, 5, 7, 8) | | | |
| Follistatin-like | anti-FSTL1 | Vimentin | pritumumab |
| protein 1 (FSTL1) | mAbs | | |
| Fucosyl-GM1 | BMS-986012 | V-domain Ig | JNJ-61610588 |
| | | suppressor of T cell | |
| | | activation (VISTA) | |
| Frizzled-10 (FZD10) | OTSA-101 | Integrin alpha- | natalizumab |
| | | 4/beta-1 | |
| GCSF-R (Also, | CSL324 | Immunoglobulin iota | anti-VPREB1 |
| CD114 and CSFR3) | | chain (VPREB1) | mAbs |
| Galectin 3 binding | MP-1959 | Wilms tumor protein | ESK1 |
| protein (LGALS3) | | (WT1/HLA); WT1 | |
| | | peptide presented in | |
| | | MHC | |
| Guanylate cyclase 2C | TAK-164 | Glypican-3 (GPC3) | codrituzumab |
| (GUCY2C) | | | |
| GD2 | dinutuximab | Transmembrane | CDX-011 |
| | | glycoprotein NMB | |
| | | (GPNMB) | |
| GD3 | PF-06688992 | Leucine-rich repeat- | BNC-101 |
| | | containing G-protein | |
| | | coupled receptor 5 | |
| | | (LGR5) | |
| glucocorticoid- | BMS-986156 | G-protein coupled | JNJ-64407564 |
| induced TNFR- | | receptor family C | |
| related protein | | group 5 member D | |
| (GITR) | | (GPRC5D) | |
| glucocorticoid- | EU-102 | Ferritin | Ferritarg P |
| induced TNFR- | | | |
| related protein | | | |
| ligand | | | |
| (GITRL) | | | |
| premelanocyte | anti-PMEL | Erbb2 tyrosine | trastuzumab; |
| protein (PMEL) | mAbs | kinase | pertuzumab; |
| | | (HER2) | margetuximab |
| Cell surface A33 | Anti-GPA33 | Erbb3 tyrosine | patritumab |
| antigen (GPA33) | mAbs | kinase | |
| | | (HER3) | |
| Glypican-1 (GPC1) | MIL-38 | Globo H | OBI-888 |

The extracellular antigen binding domain may comprise an antigen binding fragment (e.g., a scFv) derived from any of the antibodies listed in Table 1 depending upon the target antigen of interest.

In other embodiments, the extracellular antigen binding domain of any of the CAR polypeptides described herein may be specific to a pathogenic antigen, such as a bacterial antigen, a viral antigen, or a fungal antigen. Some examples are provided below: influenza virus neuraminidase, hemagglutinin, or M2 protein, human respiratory syncytial virus (RSV) F glycoprotein or G glycoprotein, herpes simplex virus glycoprotein gB, gC, gD, or gE, Chlamydia MOMP or PorB protein, Dengue virus core protein, matrix protein, or glycoprotein E, measles virus hemagglutinin, herpes simplex virus type 2 glycoprotein gB, poliovirus I VP1, envelope glycoproteins of HIV 1, hepatitis B core antigen or surface antigen, diptheria toxin, Streptococcus 24M epitope, Gonococcal pilin, pseudorabies virus g50 (gpD), pseudorabies virus II (gpB), pseudorabies virus III (gpC), pseudorabies virus glycoprotein H, pseudorabies virus glycoprotein E, transmissible gastroenteritis glycoprotein 195, transmissible gastroenteritis matrix protein, or human hepatitis C virus glycoprotein E1 or E2.

In addition, the extracellular antigen binding domain of the CAR polypeptide described herein may be specific to a tag conjugated to a therapeutic agent, which targets an antigen associated with a disease or disorder (e.g., a tumor antigen or a pathogenic antigen as described herein). In some instances, the tag conjugated to the therapeutic agent can be antigenic and the extracellular antigen binding domain of the CAR polypeptide can be an antigen-binding fragment (e.g., scFv) of an antibody having high binding affinity and/or specificity to the antigenic tag. Exemplary antigenic tags include, but are not limited to, biotin, avidin, a fluorescent molecule (e.g., GFP, YRP, luciferase, or RFP), Myc, Flag, His (e.g., poly His such as 6×His), HA (hemagglutinin), GST, MBP (maltose binding protein), KLH (keyhole limpet hemocyanins), trx, T7, HSV, VSV (e.g., VSV-G), Glu-Glu, V5, e-tag, S-tag, KT3, E2, Au1, Au5, and/or thioredoxin.

In other instances, the tag conjugated to the therapeutic agent is a member of a ligand-receptor pair and the extracellular antigen binding domain comprises the other member of the ligand-receptor pair or a fragment thereof that binds the tag. For example, the tag conjugated to the therapeutic agent can be biotin and the extracellular antigen binding domain of the CAR polypeptide can comprise a biotin-binding fragment of avidin. Sce, e.g., Urbanska et al., 2012, Lohmueller et al., 2018. Other examples include anti-Tag CAR, in which the extracellular antigen binding domain is a scFv fragment specific to a protein tag, such as FITC (Tamada et al., 2012, Kim et al., 2015; Cao et al., 2016; and Ma et al., 2016), PNE (Rodgers et al., 2016), La—SS—B (Cartellieri et al., 2016), Biotin (Lohmullular et al., 2017), and Leucine-Zipper (Cho et al., 2018). Selection of the antigen binding domain for use in the CAR polypeptides described herein will be apparent to one of skill in the art. For example, it may depend on factors such as the type of target antigen and the desired affinity of the binding interaction.

The extracellular antigen binding domain of any of the CAR polypeptides described herein may have suitable binding affinity for a target antigen (e.g., any one of the targets described herein) or antigenic epitopes thereof. As used herein, “binding affinity” refers to the apparent association constant or K_{A}. The K_{A} is the reciprocal of the dissociation constant (K_{D}). The extracellular antigen binding domain for use in the CAR polypeptides described herein may have a binding affinity (K_{D}) of at least 10⁻⁵, 10⁻⁶, 10⁻⁷, 10⁻⁸, 10⁻⁹, 10⁻¹⁰ M, or lower for the target antigen or antigenic epitope. An increased binding affinity corresponds to a decreased K_{D}. Higher affinity binding of an extracellular antigen binding domain for a first antigen relative to a second antigen can be indicated by a higher K_{A} (or a smaller numerical value K_{D}) for binding the first antigen than the K_{A} (or numerical value K_{D}) for binding the second antigen. In such cases, the extracellular antigen binding domain has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 105 fold.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

$$[ Bound ] = [ Free ] / ( Kd + [ Free ] )$$

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

### B. Transmembrane Domain

The transmembrane domain of the chimeric receptor polypeptides (e.g., ACTR polypeptides or CAR polypeptides) described herein can be in any form known in the art. As used herein, a “transmembrane domain” refers to any protein structure that is thermodynamically stable in a cell membrane, preferably a eukaryotic cell membrane. A transmembrane domain compatible for use in the chimeric receptor polypeptides used herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times).

Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini.

In some embodiments, the transmembrane domain of the chimeric receptor polypeptide described herein is derived from a Type I single-pass membrane protein.

Single-pass membrane proteins include, but are not limited to, CD8α, CD8β, 4-1BB/CD137, CD27, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B. In some embodiments, the transmembrane domain is from a membrane protein selected from the following: CD8α, CD8β, 4-1BB/CD137, CD28, CD34, CD4, FcεRIγ, CD16, OX40/CD134, CD3ζ, CD3ε, CD3γ, CD3ε, TCRα, CD32, CD64, VEGFR2, FAS, and FGFR2B. In some examples, the transmembrane domain is of CD8 (e.g., the transmembrane domain is of CD8α). In some examples, the transmembrane domain is of 4-1BB/CD137. In other examples, the transmembrane domain is of CD28. In some cases, the chimeric receptor polypeptide described herein may be free of a hinge domain from any non-CD16A receptor. In some instances, such a chimeric receptor polypeptide may be free of any hinge domain. Alternatively or in addition, such a chimeric receptor polypeptide may comprise two or more co-stimulatory regions as described herein. In other examples, the transmembrane domain is of CD34. In yet other examples, the transmembrane domain is not derived from human CD8α. In some embodiments, the transmembrane domain of the chimeric receptor polypeptide is a single-pass alpha helix.

Transmembrane domains from multi-pass membrane proteins may also be compatible for use in the chimeric receptor polypeptides described herein. Multi-pass membrane proteins may comprise a complex alpha helical structure (e.g., at least 2, 3, 4, 5, 6, 7 or more alpha helices) or a beta sheet structure. Preferably, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side. Either one or multiple helix passes from a multi-pass membrane protein can be used for constructing the chimeric receptor polypeptide described herein.

Transmembrane domains for use in the chimeric receptor polypeptides described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 B1 and PCT Publication No. WO 2000/032776 A2, the relevant disclosures of each of which are incorporated by reference herein.

In some embodiments, the amino acid sequence of the transmembrane domain does not comprise cysteine residues. In some embodiments, the amino acid sequence of the transmembrane domain comprises one cysteine residue. In some embodiments, the amino acid sequence of the transmembrane domain comprises two cysteine residues. In some embodiments, the amino acid sequence of the transmembrane domain comprises more than two cysteine residues (e.g., 3, 4, 5, or more).

The transmembrane domain may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence.

The hydropathy, hydrophobic or hydrophilic characteristics of a protein or protein segment, can be assessed by any method known in the art including, for example, the Kyte and Doolittle hydropathy analysis.

### C. Co-Stimulatory Signaling Domains

Many immune cells require co-stimulation, in addition to stimulation of an antigen-specific signal, to promote cell proliferation, differentiation and survival, as well as to activate effector functions of the cell. In some embodiments, the chimeric receptor polypeptides, such as ACTR or CAR polypeptides, described herein comprise at least one co-stimulatory signaling domain. In certain embodiments, the chimeric receptor polypeptides may contain a CD28 co-stimulatory signaling domain or a 4-1BB (CD137) co-stimulatory signaling domain. The term “co-stimulatory signaling domain,” as used herein, refers to at least a fragment of a co-stimulatory signaling protein that mediates signal transduction within a cell to induce an immune response such as an effector function (a secondary signal). As known in the art, activation of immune cells such as T cells often requires two signals: (1) the antigen specific signal (primary signal) triggered by the engagement of T cell receptor (TCR) and antigenic peptide/MHC complexes presented by antigen presenting cells, which typically is driven by CD3ζ as a component of the TCR complex; and (ii) a co-stimulatory signal (secondary signal) triggered by the interaction between a co-stimulatory receptor and its ligand. A co-stimulatory receptor transduces a co-stimulatory signal (secondary signal) as an addition to the TCR-triggered signaling and modulates responses mediated by immune cells, such as T cells, NK cells, macrophages, neutrophils, or cosinophils.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the chimeric receptor polypeptides described herein. The type(s) of co-stimulatory signaling domain is selected based on factors such as the type of the immune cells in which the chimeric receptor polypeptides would be expressed (e.g., T cells, NK cells, macrophages, neutrophils, or cosinophils) and the desired immune effector function (e.g. ADCC). Examples of co-stimulatory signaling domains for use in the chimeric receptor polypeptides may be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFRSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TLIA/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thyl, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCLIA, TCLIB, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the co-stimulatory signaling domain is of 4-1BB, CD28, OX40, ICOS, CD27, GITR, HVEM, TIM1, LFA1(CD11a) or CD2, or any variant thereof.

Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue mutations (e.g., 1, 2, 3, 4, 5, or 8) such as amino acid substitutions, deletions, or additions as compared to a wild-type counterpart. Such co-stimulatory signaling domains comprising one or more amino acid variations (e.g., amino acid substitutions, deletions, or additions) may be referred to as variants.

Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation. For example, mutation of residues 186 and 187 of the native CD28 amino acid sequence may result in an increase in co-stimulatory activity and induction of immune responses by the co-stimulatory domain of the chimeric receptor polypeptide. In some embodiments, the mutations are substitution of a lysine at each of positions 186 and 187 with a glycine residue of the CD28 co-stimulatory domain, referred to as a CD28_{LL→GG} variant. Additional mutations that can be made in co-stimulatory signaling domains that may enhance or reduce co-stimulatory activity of the domain will be evident to one of ordinary skill in the art. In some embodiments, the co-stimulatory signaling domain is of 4-1BB, CD28, OX40, or CD28_{LL→GG} variant.

In some embodiments, the chimeric receptor polypeptides may contain a single co-stimulatory domain such as, for example, a CD27 co-stimulatory domain, a CD28 co-stimulatory domain, a 4-1BB co-stimulatory domain, an ICOS co-stimulatory domain, or an OX40 co-stimulatory domain.

In some embodiments, the chimeric receptor polypeptides may comprise more than one co-stimulatory signaling domain (e.g., 2, 3, or more). In some embodiments, the chimeric receptor polypeptide comprises two or more of the same co-stimulatory signaling domains, for example, two copies of the co-stimulatory signaling domain of CD28. In some embodiments, the chimeric receptor polypeptide comprises two or more co-stimulatory signaling domains from different co-stimulatory proteins, such as any two or more co-stimulatory proteins described herein. Selection of the type(s) of co-stimulatory signaling domains may be based on factors such as the type of host cells to be used with the chimeric receptor polypeptides (e.g., T cells or NK cells) and the desired immune effector function. In some embodiments, the chimeric receptor polypeptide comprises two co-stimulatory signaling domains, for example, two copies of the co-stimulatory signaling domain of CD28. In some embodiments, the chimeric receptor polypeptide may comprise two or more co-stimulatory signaling domains from different co-stimulatory receptors, such as any two or more co-stimulatory receptors described herein, for example, CD28 and 4-1BB, CD28 and CD27, CD28 and ICOS, CD28_{LL→GG} variant and 4-1BB, CD28 and OX40, or CD28_{LL→GG} variant and OX40. In some embodiments, the two co-stimulatory signaling domains are CD28 and 4-1BB. In some embodiments, the two co-stimulatory signaling domains are CD28_{LL→GG} variant and 4-1BB. In some embodiments, the two co-stimulatory signaling domains are CD28 and OX40. In some embodiments, the two co-stimulatory signaling domains are CD28_{LL→GG} variant and OX40. In some embodiments, the chimeric receptor polypeptides described herein may contain a combination of a CD28 and ICOSL. In some embodiments, the chimeric receptor polypeptide described herein may contain a combination of CD28 and CD27. In certain embodiments, the 4-1BB co-stimulatory domain is located N-terminal to the CD28 or CD28_{LL→GG} variant co-stimulatory signaling domain.

In some embodiments, the chimeric receptor polypeptides described herein do not comprise a co-stimulatory signaling domain.

### D. Cytoplasmic Signaling Domain

Any cytoplasmic signaling domain can be used to create the chimeric receptor polypeptides described herein (e.g., ACTR polypeptides or CAR polypeptides). Such a cytoplasmic domain may be any signaling domain involved in triggering cell signaling (primary signaling) that leads to immune cell proliferation and/or activation. The cytoplasmic signaling domain as described herein is not a co-stimulatory signaling domain, which, as known in the art, relays a co-stimulatory or secondary signal for fully activating immune cells.

The cytoplasmic domain described herein may comprise an immunoreceptor tyrosine-based activation motif (ITAM) domain (e.g., at least one ITAM domain, at least two ITAM domains, or at least three ITAM domains) or may be ITAM free. An “ITAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix (6-8) YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways.

In some examples, the cytoplasmic signaling domain is of CD3ζ or FcαRIγ. In other examples, cytoplasmic signaling domain is not derived from human CD3ζ. In yet other examples, the cytoplasmic signaling domain is not derived from an Fc receptor, when the extracellular Fc-binding domain of the same chimeric receptor polypeptide is derived from CD16A.

In one specific embodiment, several signaling domains can be fused together for additive or synergistic effect. Non-limiting examples of useful additional signaling domains include part or all of one or more of TCR Zeta chain, CD28, OX40/CD134, 4-1BB/CD137, FcεRIy, ICOS/CD278, IL2R-beta/CD122, IL-2R-gamma/CD132, and CD40.

In other embodiments, the cytoplasmic signaling domain described herein is free of the ITAM motif. Examples include, but are not limited to, the cytoplasmic signaling domain of Jak/STAT, Toll-interleukin receptor (TIR), and tyrosine kinase.

### E. Hinge Domain

In some embodiments, the chimeric receptor polypeptides such as ACTR polypeptides or CAR polypeptides described herein further comprise a hinge domain that is located between the extracellular ligand-binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular ligand-binding domain relative to the transmembrane domain of the chimeric receptor polypeptide can be used.

Hinge domains of any protein known in the art to comprise a hinge domain are compatible for use in the chimeric receptor polypeptides described herein. In some embodiments, the hinge domain is at least a portion of a hinge domain of a naturally occurring protein and confers flexibility to the chimeric receptor polypeptide. In some embodiments, the hinge domain is of CD8. In some embodiments, the hinge domain is a portion of the hinge domain of CD8, e.g., a fragment containing at least 15 (e.g., 20, 25, 30, 35, or 40) consecutive amino acids of the hinge domain of CD8. In some embodiments, the hinge domain is of CD28. In some embodiments, the hinge domain is a portion of the hinge domain of CD28, e.g., a fragment containing at least 15 (e.g., 20, 25, 30, 35, or 40) consecutive amino acids of the hinge domain of CD28. The hinge domain and/or the transmembrane domain may be linked to additional amino acids (e.g., 15 aa, 10-aa, 8-aa, 6-aa, or 4-aa) at the N-terminal portion, at the C-terminal portion, or both. Examples can be found, e.g., in Ying et al., Nature Medicine, 25 (6): 947-953 (2019).

In some embodiments, the hinge domain is of CD16A receptor, for example, the whole hinge domain of a CD16A receptor or a portion thereof, which may consists of up to 40 consecutive amino acid residues of the CD16A receptor (e.g., 20, 25, 30, 35, or 40). Such a chimeric receptor polypeptide (e.g., an ACTR polypeptide) may contain no hinge domain from a different receptor (a non-CD16A receptor).

Hinge domains of antibodies, such as an IgG, IgA, IgM, IgE, or IgD antibodies, are also compatible for use in the chimeric receptor polypeptides described herein. In some embodiments, the hinge domain is the hinge domain that joins the constant domains CHI and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the chimeric receptor polypeptides described herein. In some embodiments, the hinge domain between the C-terminus of the extracellular target-binding domain and the N-terminus of the transmembrane domain is a peptide linker, such as a (GlyₓSer)ₙ linker, wherein x and n, independently can be an integer between 3 and 12, including 3, 4, 5, 6, 7, 8, 9, 10, 11, 12, or more. In some embodiments, the hinge domain is (Gly₄Ser)ₙ (SEQ ID NO:95), wherein n can be an integer between 3 and 60, including 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60. In certain embodiments, n can be an integer greater than 60. In some embodiments, the hinge domain is (Gly₄Ser)₃ (SEQ ID NO: 96). In some embodiments, the hinge domain is (Gly₄Ser)₆ (SEQ ID NO: 97). In some embodiments, the hinge domain is (Gly₄Ser)₉ (SEQ ID NO: 98). In some embodiments, the hinge domain is (Gly₄Ser)₁₂ (SEQ ID NO: 99). In some embodiments, the hinge domain is (Gly₄Ser)₁₅ (SEQ ID NO: 100). In some embodiments, the hinge domain is (Gly₄Ser)₃₀ (SEQ ID NO: 101). In some embodiments, the hinge domain is (Gly₄Ser)₄₅ (SEQ ID NO: 102). In some embodiments, the hinge domain is (Gly₄Ser)₆₀ (SEQ ID NO: 103).

In other embodiments, the hinge domain is an extended recombinant polypeptide (XTEN), which is an unstructured polypeptide consisting of hydrophilic residues of varying lengths (e.g., 10-80 amino acid residues). Amino acid sequences of XTEN peptides will be evident to one of skill in the art and can be found, for example, in U.S. Pat. No. 8,673,860, the relevant disclosures of which are incorporated by reference herein. In some embodiments, the hinge domain is an XTEN peptide and comprises 60 amino acids. In some embodiments, the hinge domain is an XTEN peptide and comprises 30 amino acids. In some embodiments, the hinge domain is an XTEN peptide and comprises 45 amino acids. In some embodiments, the hinge domain is an XTEN peptide and comprises 15 amino acids.

Any of the hinge domains used for making the chimeric receptor polypeptide as described herein may contain up to 250 amino acid residues. In some instances, the chimeric receptor polypeptide may contain a relatively long hinge domain, for example, containing 150-250 amino acid residues (e.g., 150-180 amino acid residues, 180-200 amino acid residues, or 200-250 amino acid residues). In other instances, the chimeric receptor polypeptide may contain a medium sized hinge domain, which may contain 60-150 amino acid residues (e.g., 60-80, 80-100, 100-120, or 120-150 amino acid residues). Alternatively, the chimeric receptor polypeptide may contain a short hinge domain, which may contain less than 60 amino acid residues (e.g., 1-30 amino acids or 31-60 amino acids). In some embodiments, a chimeric receptor polypeptide (e.g., an ACTR polypeptide) described herein contains no hinge domain or no hinge domain from a non-CD16A receptor.

### F. Signal Peptide

In some embodiments, the chimeric receptor polypeptide (e.g., ACTR polypeptide or CAR polypeptide) may also comprise a signal peptide (also known as a signal sequence) at the N-terminus of the polypeptide. In general, signal sequences are peptide sequences that target a polypeptide to the desired site in a cell. In some embodiments, the signal sequence targets the chimeric receptor polypeptide to the secretory pathway of the cell and will allow for integration and anchoring of the chimeric receptor polypeptide into the lipid bilayer. Signal sequences including signal sequences of naturally occurring proteins or synthetic, non-naturally occurring signal sequences that are compatible for use in the chimeric receptor polypeptides described herein will be evident to one of skill in the art. In some embodiments, the signal sequence from CD8α. In some embodiments, the signal sequence is from CD28. In other embodiments, the signal sequence is from the murine kappa chain. In yet other embodiments, the signal sequence is from CD16.

### G. Examples of ACTR Polypeptides

Exemplary ACTR constructs for use with the methods and compositions described herein may be found, for example, in the instant description and figures or may be found in PCT Patent Publication No.: WO2016040441A1, WO2017/161333, and PCT Application No.: PCT/US2018/015999, each of which is incorporated by reference herein for this purpose. The ACTR polypeptides described herein may comprise a CD16A extracellular domain with binding affinity and specificity for the Fc portion of an IgG molecule, a transmembrane domain, and a CD3ζ cytoplasmic signaling domain. In some embodiments, the ACTR polypeptides may further include one or more co-stimulatory signaling domains, one of which may be a CD28 co-stimulatory signaling domain or a 4-1BB co-stimulatory signaling domain. The ACTR polypeptides are configured such that, when expressed on a host cell, the extracellular ligand-binding domain is located extracellularly for binding to a target molecule and the CD3ζ cytoplasmic signaling domain. The co-stimulatory signaling domain may be located in the cytoplasm for triggering activation and/or effector signaling.

In some embodiments, an ACTR polypeptide as described herein may comprise, from N-terminus to C-terminus, the Fc binding domain such as a CD16A extracellular domain, the transmembrane domain, the optional one or more co-stimulatory domains (e.g., a CD28 co-stimulatory domain, a 4-1BB co-stimulatory signaling domain, an OX40 co-stimulatory signaling domain, a CD27 co-stimulatory signaling domain, or an ICOS co-stimulatory signaling domain), and the CD3ζ cytoplasmic signaling domain.

Alternatively or in addition, the ACTR polypeptides described herein may contain two or more co-stimulatory signaling domains, which may link to each other or be separated by the cytoplasmic signaling domain. The extracellular Fc binder, transmembrane domain, optional co-stimulatory signaling domain(s), and cytoplasmic signaling domain in an ACTR polypeptide may be linked to each other directly, or via a peptide linker. In some embodiments, any of the ACTR polypeptides described herein may comprise a signal sequence at the N-terminus.

Table 4 provides exemplary ACTR polypeptides described herein. These exemplary constructs have, from N-terminus to C-terminus in order, the signal sequence, the Fc binding domain (e.g., an extracellular domain of an Fc receptor), the hinge domain, and the transmembrane, while the positions of the optional co-stimulatory domain and the cytoplasmic signaling domain can be switched.

TABLE 4
Exemplary Components of ACTR polypeptides.

| Exemplary | | Extracellular | | Trans- | Co- | Cytoplasmic |
|---|---|---|---|---|---|---|
| AA Sequence | Signal | domain of Fc | Hinge | membrane | stimulatory | Signaling |
| (SEQ ID NO) | Sequence | receptor | domain | domain | domain | domain |
| 1 | CD8α | CD16A-V158 | CD8α | CD8α | 4-1BB | CD3ζ |
| | | | | | (CD137) | |
| 2 | CD8α | CD16A-V158 | CD8α | 4-1BB | 4-1BB | CD3ζ |
| | | | | (CD137) | (CD137) | |
| 3 | CD8α | CD16A-V158 | CD8α | CD28 | 4-1BB | CD3ζ |
| | | | | | (CD137) | |
| 4 | CD8α | CD16A-V158 | CD8α | CD34 | 4-1BB | CD3ζ |
| | | | | | (CD137) | |
| 5 | CD8α | CD16A-V158 | CD8α | Designed | 4-1BB | CD3ζ |
| | | | | hydrophobic | (CD137) | |
| | | | | TM domain | | |
| 6 | CD8α | CD32A | CD8α | CD8α | 4-1BB | CD3ζ |
| | | | | | (CD137) | |
| 7 | CD8α | CD16A-V158 | CD8α | CD8α | CD28 | CD3ζ |
| 8 | CD8α | CD16A-V158 | CD8α | CD8α | OX40 | CD3ζ |
| | | | | | (CD134) | |
| 9 | CD8α | CD16A-V158 | CD8α | CD8α | CD28 + | CD3ζ |
| | | | | | 4-1BB | |
| 10 | CD8α | CD16A-V158 | None | CD8α | 4-1BB | CD3ζ |
| | | | | | (CD137) | |
| 11 | CD8α | CD16A-V158 | XTEN | CD8α | 4-1BB | CD3ζ |
| | | | | | (CD137) | |
| 12 | CD8α | CD16A-V158 | CD8α | CD8α | CD28 LL to | CD3ζ |
| | | | | | GG mutant | |
| 13 | CD8α | CD16A-V158 | CD8α | CD8α | CD28 LL to | CD3ζ |
| | | | | | GG mutant + | |
| | | | | | 4-1BB | |
| 14 | CD8α | CD16A-V158 | CD8α | CD4 | 4-1BB | CD3ζ |
| | | | | | (CD137) | |
| 15 | CD8α | CD16A-V158 | CD8α | CD4 | CD28 LL to | CD3ζ |
| | | | | | GG mutant + | |
| | | | | | 4-1BB | |
| 16 | CD8α | CD16A-V158 | CD8α | FcεRIγ | 4-1BB | CD3ζ |
| | | | | | (CD137) | |
| 17 | CD8α | CD16A-V158 | CD8α | Designed | 4-1BB | CD3ζ |
| | | | | hydrophobic | (CD137) | |
| | | | | TM domain, | | |
| | | | | predicted | | |
| | | | | dimerization | | |
| 18 | CD8α | CD16A-V158 | CD8α | CD8β | 4-1BB | CD3ζ |
| | | | | | (CD137) | |
| 19 | CD8α | CD16A-V158 | CD8α | C16α | 4-1BB | CD3ζ |
| | | | | | (CD137) | |
| 20 | CD8α | CD16A-V158 | CD8α | OX40 | 4-1BB | CD3ζ |
| | | | | (CD134) | (CD137) | |
| 21 | CD8α | CD16A-V158 | CD8α | CD3ζ | 4-1BB | CD3ζ |
| | | | | | (CD137) | |
| 22 | CD8α | CD16A-V158 | CD8α | CD3ε | 4-1BB | CD3ζ |
| | | | | | (CD137) | |
| 23 | CD8α | CD16A-V158 | CD8α | CD3γ | 4-1BB | CD3ζ |
| | | | | | (CD137) | |
| 24 | CD8α | CD16A-V158 | CD8α | CD3δ | 4-1BB | CD3ζ |
| | | | | | (CD137) | |
| 25 | CD8α | CD16A-V158 | CD8α | TCR-α | 4-1BB | CD3ζ |
| | | | | | (CD137) | |
| 26 | CD8α | CD16A-V158 | CD8α | CD32 | 4-1BB | CD3ζ |
| | | | | | (CD137) | |
| 27 | CD8α | CD16A-V158 | CD8α | CD64 | 4-1BB | CD3ζ |
| | | | | | (CD137) | |
| 28 | CD8α | CD16A-V158 | CD8α | VEGFR2 | 4-1BB | CD3ζ |
| | | | | | (CD137) | |
| 29 | CD8α | CD16A-V158 | CD8α | FAS | 4-1BB | CD3ζ |
| | | | | | (CD137) | |
| 30 | CD8α | CD16A-V158 | CD8α | FGFR2B | 4-1BB | CD3ζ |
| | | | | | (CD137) | |
| 31 | CD8α | CD16A-F158 | CD8α | CD8α | 4-1BB | CD3ζ |
| | | | | | (CD137) | |
| 32 | CD8α | CD64A | CD8α | CD8α | 4-1BB | CD3ζ |
| | | | | | (CD137) | |
| 33 | CD8α | CD16A-V158 | IgG1 | CD8α | 4-1BB | CD3ζ |
| | | | (hinge- | | (CD137) | |
| | | | CH2-CH3) | | | |
| 34 | CD8α | CD16A-V158 | IgG1 | CD8α | 4-1BB | CD3ζ |
| | | | (hinge- | | (CD137) | |
| | | | CH3) | | | |
| 35 | CD8α | CD16A-V158 | IgG1 | CD8α | 4-1BB | CD3ζ |
| | | | (hinge) | | (CD137) | |
| 36 | CD8α | CD16A-V158 | CD8- | CD8α | 4-1BB | CD3ζ |
| | | | alpha | | (CD137) | |
| | | | fragment 1 | | | |
| | | | (30 amino | | | |
| | | | acids) | | | |
| 37 | CD8α | CD16A-V158 | CD8- | CD8α | 4-1BB | CD3ζ |
| | | | alpha | | (CD137) | |
| | | | fragment 2 | | | |
| | | | (15 amino | | | |
| | | | acids) | | | |
| 38 | CD8α | CD16A-V158 | (Gly4Ser) × 3 | CD8α | 4-1BB | CD3ζ |
| | | | (60 amino | | (CD137) | |
| | | | acids) | | | |
| 39 | CD8α | CD16A-V158 | (Gly4Ser) × 6 | CD8α | 4-1BB | CD3ζ |
| | | | (45 amino | | (CD137) | |
| | | | acids) | | | |
| 40 | CD8α | CD16A-V158 | (Gly4Ser) × 9 | CD8α | 4-1BB | CD3ζ |
| | | | (30 amino | | (CD137) | |
| | | | acids) | | | |
| 41 | CD8α | CD16A-V158 | (Gly4Ser) × 12 | CD8α | 4-1BB | CD3ζ |
| | | | (15 amino | | (CD137) | |
| | | | acids) | | | |
| 42 | CD8α | CD16A-V158 | XTEN | CD8α | 4-1BB | CD3ζ |
| | | | (60 amino | | (CD137) | |
| | | | acids) | | | |
| 43 | CD8α | CD16A-V158 | XTEN | CD8α | 4-1BB | CD3ζ |
| | | | (30 amino | | (CD137) | |
| | | | acids) | | | |
| 44 | CD8α | CD16A-V158 | XTEN | CD8α | 4-1BB | CD3ζ |
| | | | (15 amino | | (CD137) | |
| | | | acids) | | | |
| 45 | CD28 | CD16A-V158 | CD8α | CD8α | 4-1BB | CD3ζ |
| | | | | | (CD137) | |
| 46 | Murine | CD16A-V158 | CD8α | CD8α | 4-1BB | CD3ζ |
| | kappa | | | | (CD137) | |
| | chain | | | | | |
| 47 | CD16 | CD16A-V158 | CD8α | CD8α | 4-1BB | CD3ζ |
| | | | | | (CD137) | |
| 48 | CD8α | CD16A-V158 | CD8α | CD8α | ICOS | CD3ζ |
| 49 | CD8α | CD16A-V158 | CD8α | CD8α | CD27 | CD3ζ |
| 50 | CD8α | CD16A-V158 | CD8α | CD8α | GITR | CD3ζ |
| 51 | CD8α | CD16A-V158 | CD8α | CD8α | HVEM | CD3ζ |
| 52 | CD8α | CD16A-V158 | CD8α | CD8α | TIM1 | CD3ζ |
| 53 | CD8α | CD16A-V158 | CD8α | CD8α | LFA1 | CD3ζ |
| | | | | | (CD11a) | |
| 54 | CD8α | CD16A-V158 | CD8α | CD8α | CD2 | CD3ζ |
| 55 | CD8α | CD16A-V158 | CD8α | FcεR1γ | 4-1BB | FcεR1γ |
| | | | | | (CD137) | |
| 56 | CD8α | CD16A-V158 | CD8α | CD8α | 4-1BB | FcεR1γ |
| | | | | | (CD137) | |
| 57 | CD8α | CD16A-V158 | CD28 | CD28 | CD28 | CD3ζ |
| | | | (e.g., | | | |
| | | | 39aa) | | | |
| 58 | CD8α | CD16A-V158 | none | CD8 | CD28 | CD3ζ |
| 59 | CD8α | CD16A-V158 | CD8 | CD8 | CD28 + | CD3ζ |
| | | | | | CD27 | |
| 60 | CD8α | CD16A-V158 | CD8 | CD8 | CD28 + | CD3ζ |
| | | | | | OX40 | |
| 61 | CD8α | CD16A-V158 | CD8 | CD8 | 4-1BB + | CD3ζ |
| | | | | | CD28 | |
| 62 | CD8α | CD16A-V158 | CD28 | CD28 | CD28 + | CD3ζ |
| | | | | | 4-1BB | |
| 63 | CD8α | CD16A-V158 | CD28 | CD28 | 4-1BB | CD3ζ |
| 64 | CD8α | CD16A-V158 | CD8 | CD8 | CD27 | CD3ζ |
| 65 | CD8α | CD16A-V158 | CD8 | CD8 | CD28 | CD3ζ |
| 66 | CD8α | CD16A-V158 | CD8 | CD8 | ICOS | CD3ζ |
| 67 | CD8α | CD16A-V158 | CD8 | CD8 | OX40 | CD3ζ |
| 68 | CD8α | CD16A-V158 | CD8 | CD8 | CD28 and | CD3ζ |
| | | | | | ICOS | |
| 69 | CD8α | CD16A-V158 | none | CD8 | 4-1BB | CD3ζ |
| 70 | CD8α | CD16A-V158 | none | CD8 | CD27 | CD3ζ |
| 71 | CD8α | CD16A-V158 | none | CD8 | ICOS | CD3ζ |
| 72 | CD8α | CD16A-V158 | none | CD8 | OX40 | CD3ζ |
| 73 | CD8α | CD16A-V158 | none | CD8 + 4aa | 4-1BB | CD3ζ |
| 74 | CD8α | CD16A-V158 | none | CD8 + 4aa | CD28 | CD3ζ |
| 75 | CD8α | CD16A-V158 | CD8 | CD28 | CD28 | CD3ζ |
| 76 | CD8α | CD16A-V158 | CD28 | CD28 | CD28 | CD3ζ |
| | | | (26aa) | | | |
| 77 | CD8α | CD16A-V158 | CD28 | CD28 | CD28 | CD3ζ |
| | | | (16aa) | | | |
| 78 | CD8α | CD16A-V158 | none | CD28 | CD28 | CD3ζ |
| 79 | CD8α | CD16A-V158 | CD8 | CD8 | 41BB | CD3ζ |
| 80 | CD8α | CD16A-V158 | CD28 | CD8 | CD28 | CD3ζ |
| | | | (39 aa) | | | |

Amino acid sequences of the example ACTR polypeptides are provided below (signal sequence italicized).

SEQ ID NO: 1:
MALPVTALLLPLALLLHAARPGMRTEDLPKAVVFLEPQWYRVLEKDSVTLKCQGAYSPEDNSTQWFHNE
SLISSQASSYFIDAATVDDSGEYRCQTNLSTLSDPVQLEVHIGWLLLQAPRWVFKEEDPIHLRCHSWKN
TALHKVTYLQNGKGRKYFHHNSDFYIPKATLKDSGSYFCRGLVGSKNVSSETVNITITQGLAVSTISSFF
PPGYQTTTPAPRPPTPAPTIASQPLSLRPEACRPAAGGAVHTRGLDFACDIYIWAPLAGTCGVLLLSLVIT
LYCKRGRKKLLYIFKQPFMRPVQTTQEEDGCSCRFPEEEEGGCELRVKFSRSADAPAYQQGQNQLYNE
LNLGRREEYDVLDKRRGRDPEMGGKPRRKNPQEGLYNELQKDKMAEAYSEIGMKGERRRGKGHDGL
YQGLSTATKDTYDALHMQALPPR
SEQ ID NO: 2:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIISFFLALTST
ALLFLLFFLTLRFSVVKRGKRGRKKLLYIFKQPFMRPVQTTQEEDGCSCRFPEEEEGGCELRVKESRS
ADAPAYQQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
SEQ ID NO: 3:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDFWVLVVVGG
VLACYSLLVTVAFIIFWVRSKKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR
SEQ ID NO: 4:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDLIALVTSGAL
LAVLGITGYFLMNRKRGRKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 5:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDLLAALLALLA
ALLALLAALLARSKKRGRKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 6:
MALPVTALLLPLALLLHAARPQAAAPPKAVLKLEPPWINVLQEDSVTLTCQGARSPESDSIQWFHNGN
LIPTHTQPSYRFKANNNDSGEYTCQTGQTSLSDPVHLTVLSEWLVLQTPHLEFQEGETIMLRCHSWK
DKPLVKVTFFQNGKSQKFSHLDPTFSIPQANHSHSGDYHCTGNIGYTLFSSKPVTITVQVPSMGSSSP
MGTTTPAPRPPTPAPTIASQPLSLRPEACRPAAGGAVHTRGLDFACDIYIWAPLAGTCGVLLLSLVIT
LYCKRGRKKLLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGMKGERRRGKG
HDGLYQGLSTATKDTYDALHMQALPPR
SEQ ID NO: 7:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCRSKRSRLLHSDYMNMTPRRPGPTRKHYQPYAPPRDFAAYRSRVKFSRSADAP
AYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 8:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCALYLLRRDQRLPPDAHKPPGGGSFRTPIQEEQADAHSTLAKIRVKFSRSADAPA
YQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 9:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCRSKRSRLLHSDYMNMTPRRPGPTRKHYQPYAPPRDFAAYRSKRGRKKLLYIF
KQPFMRPVQTTQEEDGCSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRGKGHDGLYQGLSTATK
DTYDALHMQALPPR
SEQ ID NO: 10:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQIYIWAPLAGTCGVLLLSLVITLYCKRGRKKLLYIFKQPFMRPVQTTQEEDGCSC
RFPEEEEGGCELRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR
SEQ ID NO: 11:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQGGSPAGSPTSTEEGTSESATPESGPGTSTEPSEGSAPGSPAGSPTIYIWAPLAGTC
GVLLLSLVITLYCKRGRKKLLYIFKQPFMRPVQTTQEEDGCSCRFPEEEEGGCELRVKFSRSADAPA
YQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 12:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCRSKRSRGGHSDYMNMTPRRPGPTRKHYQPYAPPRDFAAYRSRVKFSRSADAP
AYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 13:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCRSKRSRGGHSDYMNMTPRRPGPTRKHYQPYAPPRDFAAYRSKRGRKKLLYIF
KQPFMRPVQTTQEEDGCSCRFPEEEEGGCELRVKFSRSADAPAYQQGQNQLYNELNLGRREEYDV
LDKRRGRDPEMGGKPRRKNPQEGLYNELQKDKMAEAYSEIGMKGERRRGKGHDGLYQGLSTATK
DTYDALHMQALPPR
SEQ ID NO: 14:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDMALIVLGGV
AGLLLFIGLGIFFCVRKRGRKKLLYIFKQPFMRPVQTTQEEDGCCRFPEEEEGGCELRVKFSRSADA
PAYQQGQNQLYNELNLGRREEYDVLDKRRGRDPEMGGKPRRKNPQEGLYNELQKDKMAEAYSEI
GMKGERRRGKGHDGLYQGLSTATKDTYDALHMQALPPR
SEQ ID NO: 15:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDMALIVLGGV
AGLLLFIGLGIFFCVRRSKRSRGGHSDYMNMTPRRPGPTRKHYQPYAPPRDFAAYRSRVKFSRSAD
APAYQQGQNQLYNELNLGRREEYDVLDKRRGRDPEMGGKPRRKNPQEGLYNELQKDKMAEAYS
EIGMKGERRRGKGHDGLYQGLSTATKDTYDALHMQALPPR
SEQ ID NO: 16:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDLCYILDAILFL
YGIVLTLLYCRLKKRGRKKLLYIFKQPFMRPVQTTQEEDGCSCRFPEEEEGGCELRVKFSRSADAPA
YQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 17:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDLLLILLGVLA
GVLATLAALLARSKKRGRKKLLYIFKQPFMRPVQTTQEEDGCSCRFPEEEEGGCELRVKFSRSADA
PAYQQGQNQLYNELNLGRREEYDVLDKRRGRDPEMGGKPRRKNPQEGLYNELQKDKMAEAYSEI
GMKGERRRGKGHDGLYQGLSTATKDTYDALHMQALPPR
SEQ ID NO: 18:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDITLGLLVAGV
LVLLVSLGVAIHLCKRGRKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 19:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDVSFCLVMVL
LFAVDTGLYFSVKTNKRGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLYNELQKDKMAEAYS
EIGMKGERRRGKGHDGLYQGLSTATKDTYDALHMQALPPR
SEQ ID NO: 20:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDVAAILGLGLV
LGLLGPLAILLALYKRGRKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 21:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDLCYLLDGILFI
YGVILTALFLRVKKRGRKKLLYIFKQPFMRPVQTTQEEDGCSCRFPEEEEGGCELRVKFSRSADAPA
YQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 22:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDVMSVATIVIV
DICITGGLLLLVYYWSKNRKRGRKKLLYIFKQPFMRPVQTTQEEDGCSCRFPEEEEGGCELRVKFSR
SADAPAYQQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR
SEQ ID NO: 23:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDGFLFAEIVSIF
VLAVGVYFIAGQDKRGRKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 24:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDGIIVTDVIATL
LLALGVFCFAGHETKRGRKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 25:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDVIGFRILLLK
VAGFNLLMTLRLWKRGRKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 26:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIIVAVVIATA
VAAIVAAVVALIYCRKKRGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLYNELQKDKMAEAYS
EIGMKGERRRGKGHDGLYQGLSTATKDTYDALHMQALPPR
SEQ ID NO: 27:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDVLFYLAVGI
MFLVNTVLWVTIRKEKRGRKKLLYIFKQPFMRPVQTTQEEDGCSCRFPEEEEGGCELRVKFSRSAD
APAYQQGQNQLYNELNLGRREEYDVLDKRRGRDPEMGGKPRRKNPQEGLYNELQKDKMAEAYS
EIGMKGERRRGKGHDGLYQGLSTATKDTYDALHMQALPPR
SEQ ID NO: 28:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIIILVGTAVIA
MFFWLLLVIILRTKRGRKKLLYIFKQPFMRPVQTTQEEDGCSCRFPEEEEGGCELRVKFSRSADAPA
YQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 29:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDLGWLCLLLLP
IPLIVWVKRKKRGRKKLLYIFKQPFMRPVQTTQEEDGCSCRFPEEEEGGCELRVKFSRSADAPAYQQ
GQNQLYNELNLGRREEYDVLDKRRGRDPEMGGKPRRKNPQEGLYNELQKDKMAEAYSEIGMKGE
RRRGKGHDGLYQGLSTATKDTYDALHMQALPPR
SEQ ID NO: 30:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIAIYCIGVFLI
ACMVVTVILCRMKKRGRKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 31:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLFGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCKRGRKKLLYIFKQPFMRPVQTTQEEDGCCRFPEEEEGGCELRVKFSRSADAP
AYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 32:
MALPVTALLLPLALLLHAARPQVDTTKAVITLQPPWVSVFQEETVTLHCEVLHLPGSSSTQWFLNGTA
TQTSTPSYRITSASVNDSGEYRCQRGLSGRSDPIQLEIHRGWLLLQVSSRVFTEGEPLALRCHAWKD
KLVYNVLYYRNGKAFKFFHWNSNLTILKTNISHNGTYHCSGMGKHRYTSAGISVTVKELFPAPVLN
ASVTSPLLEGNLVTLSCETKLLLQRPGLQLYFSFYMGSKTLRGRNTSSEYQILTARREDSGLYWCEA
ATEDGNVLKRSPELELQVLGLQLPTPVWFHIYIWAPLAGTCGVLLLSLVITLYCKRGRKKLLYIFKQ
PFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDT
YDALHMQALPPR
SEQ ID NO: 33:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGKIYIWAPLAGTCGVLLLSLVIT
LYCKRGRKKLLYIFKQPFMRPVQTTQEEDGCSCRFPEEEEGGCELRVKFSRSADAPAYQQGQNQLY
NELNLGRREEYDVLDKRRGRDPEMGGKPRRKNPQEGLYNELQKDKMAEAYSEIGMKGERRRGKG
HDGLYQGLSTATKDTYDALHMQALPPR
SEQ ID NO: 34:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQEPKSCDKTHTCPGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNHYTQKSLSLSPGKI
YIWAPLAGTCGVLLLSLVITLYCKRGRKKLLYIFKQPFMRPVQTTQEEDGCSCRFPEEEEGGCELRV
KFSRSADAPAYQQGQNQLYNELNLGRREEYDVLDKRRGRDPEMGGKPRRKNPQEGLYNELQKDK
MAEAYSEIGMKGERRRGKGHDGLYQGLSTATKDTYDALHMQALPPR
SEQ ID NO: 35:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQEPKSCDKTHTCPIYIWAPLAGTCGVLLLSLVITLYCKRGRKKLLYIFKQPFMRPV
QTTQEEDGCSCRFPEEEEGGCELRVKFSRSADAPAYQQGQNQLYNELNLGRREEYDVLDKRRGRD
PEMGGKPRRKNPQEGLYNELQKDKMAEAYSEIGMKGERRRGKGHDGLYQGLSTATKDTYDALH
MQALPPR
SEQ ID NO: 36:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEAFACDIYIWAPLAGTCGVLLLSLVITLYCKR
GRKKLLYIFKQPFMRPVQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGMKGERRRGKGHDGL
YQGLSTATKDTYDALHMQALPPR
SEQ ID NO: 37:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPFACDIYIWAPLAGTCGVLLLSLVITLYCKRGRKKLLYIFKQPFM
RPVQTTQEEDGCCRFPEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDGLYQGLSTATKDTYD
ALHMQALPPR
SEQ ID NO: 38:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQGGGGSGGGGSGGGGSIYIWAPLAGTCGVLLLSLVITLYCKRGRKKLLYIFKQPF
MRPVQTTQEEDGCSCRFPEEEEGGCELRVKFSRSADAPAYQQGQNQLYNELNLGRREEYDVLDKR
RGRDPEMGGKPRRKNPQEGLYNELQKDKMAEAYSEIGMKGERRRGKGHDGLYQGLSTATKDTYD
ALHMQALPPR
SEQ ID NO: 39:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQGGGGSGGGGSGGGGSGGGGSGGGGSGGGGSIYIWAPLAGTCGVLLLSLVITLY
CKRGRKKLLYIFKQPFMRPVQTTQEEDGCSCRFPEEEEGGCELRVKFSRSADAPAYQQGQNQLYNE
LNLGRREEYDVLDKRRGRDPEMGGKPRRKNPQEGLYNELQKDKMAEAYSEIGMKGERRRGKGHD
GLYQGLSTATKDTYDALHMQALPPR
SEQ ID NO: 40:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQGGGGSGGGGSGGGGSGGGGSGGGGSGGGGSGGGGSGGGGSGGGGSIYIWAPL
AGTCGVLLLSLVITLYCKRGRKKLLYIFKQPFMRPVQTTQEEDGCSCRFPEEEEGGCELRVKFSRSA
DAPAYQQGQNQLYNELNLGRREEYDVLDKRRGRDPEMGGKPRRKNPQEGLYNELQKDKMAEAY
SEIGMKGERRRGKGHDGLYQGLSTATKDTYDALHMQALPPR
SEQ ID NO: 41:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQGGGGSGGGGSGGGGSGGGGSGGGGSGGGGSGGGGSGGGGSGGGGSGGGGSG
GGGSGGGGSIYIWAPLAGTCGVLLLSLVITLYCKRGRKKLLYIFKQPFMRPVQTTQEEDGCSCRFPE
EEEGGCELRVKFSRSADAPAYQQGQNQLYNELNLGRREEYDVLDKRRGRDPEMGGKPRRKNPQE
GLYNELQKDKMAEAYSEIGMKGERRRGKGHDGLYQGLSTATKDTYDALHMQALPPR
SEQ ID NO: 42:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQGGSPAGSPTSTEEGTSESATPESGPGTSTEPSEGSAPGSPAGSPTSTEEGTSTEPSE
GSAIYIWAPLAGTCGVLLLSLVITLYCKRGRKKLLYIFKQPFMRPVQTTQEEDGCSCRFPEEEEGGCE
LRVKFSRSADAPAYQQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR
SEQ ID NO: 43:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQGGSPAGSPTSTEEGTSESATPESGPGTSTEIYIWAPLAGTCGVLLLSLVITLYCKR
GRKKLLYIFKQPFMRPVQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGMKGERRRGKGHDGL
YQGLSTATKDTYDALHMQALPPR
SEQ ID NO: 44:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQGGSPAGSPTSTEEGTIYIWAPLAGTCGVLLLSLVITLYCKRGRKKLLYIFKQPFM
RPVQTTQEEDGCSCRFPEEEEGGCELRVKFSRSADAPAYQQGQNQLYNELNLGRREEYDVLDKRR
GRDPEMGGKPRRKNPQEGLYNELQKDKMAEAYSEIGMKGERRRGKGHDGLYQGLSTATKDTYD
ALHMQALPPR
SEQ ID NO: 45:
MLRLLLALNLFPSIQVTGGMRTEDLPKAVVFLEPQWYRVLEKDSVTLKCQGAYSPEDNSTQWFHNE
SLISSQASSYFIDAATVDDSGEYRCQTNLSTLSDPVQLEVHIGWLLLQAPRWVFKEEDPIHLRCHSW
KNTALHKVTYLQNGKGRKYFHHNSDFYIPKATLKDSGSYFCRGLVGSKNVSSETVNITITQGLAVS
TISSFFPPGYQTTTPAPRPPTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGGCELRVKFSRSADAPAYQ
QGQNQLYNELNLGRREEYDVLDKRRGRDPEMGGKPRRKNPQEGLYNELQKDKMAEAYSEIGMK
GERRRGKGHDGLYQGLSTATKDTYDALHMQALPPR
SEQ ID NO: 46:
METDTLLLWVLLLWVPGSTGDGMRTEDLPKAVVFLEPQWYRVLEKDSVTLKCQGAYSPEDNSTQW
FHNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 47:
MWQLLLPTALLLLVSAGMRTEDLPKAVVFLEPQWYRVLEKDSVTLKCQGAYSPEDNSTQWFHNESL
ISSQASSYFIDAATVDDSGEYRCQTNLSTLSDPVQLEVHIGWLLLQAPRWVFKEEDPIHLRCHSWKN
TALHKVTYLQNGKGRKYFHHNSDFYIPKATLKDSGSYFCRGLVGSKNVSSETVNITITQGLAVSTIS
SFFPPGYQTTTPAPRPPTPAPTIASQPLSLRPEACRPAAGGAVHTRGLDFACDIYIWAPLAGTCGVLL
LSLVITLYCKRGRKKLLYIFKQPFMRPVQTTQEEDGCSCRFPEEEEGGCELRVKFSRSADAPAYQQG
QNQLYNELNLGRREEYDVLDKRRGRDPEMGGKPRRKNPQEGLYNELQKDKMAEAYSEIGMKGER
RRGKGHDGLYQGLSTATKDTYDALHMQALPPR
SEQ ID NO: 48:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCCWLTKKKYSSSVHDPNGEYMFMRAVNTAKKSRLTDVTLRVKFSRSADAPAY
QQGQNQLYNELNLGRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 49:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCQRRKYRSNKGESPVEPAEPCRYSCPREEEGSTIPIQEDYRKPEPACSPRVKFSRS
ADAPAYQQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
SEQ ID NO: 50:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCQLGLHIWQLRSQCMWPRETQLLLEVPPSTEDARSCQFPEEERGERSAEEKGRL
GDLWVRVKFSRSADAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR
SEQ ID NO: 51:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCCVKRRKPRGDVVKVIVSVQRKRQEAEGEATVIEALQAPPDVTTVAVEETIPSF
TGRSPNHRVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
SEQ ID NO: 52:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCKKYFFKKEVQQLSVSFSSLQIKALQNAVEKEVQAEDNIYIENSLYATDRVKFS
RSADAPAYQQGQNQLYNELNLGRREEYDVLDKRRGRDPEMGGKPRRKNPQEGLYNELQKDKMA
EAYSEIGMKGERRRGKGHDGLYQGLSTATKDTYDALHMQALPPR
SEQ ID NO: 53:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCYKVGFFKRNLKEKMEAGRGVPNGIPAEDSEQLASGQEAGDPGCLKPLHEKDS
ESGGGKDRVKFSRSADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQALPPR
SEQ ID NO: 54:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCKRKKQRSRRNDEELETRAHRVATEERGRKPHQIPASTPQNPATSQHPPPPPGH
RSQAPSHRPPPPGHRVQHQPQKRPPAPSGTQVHQQKGPPLPRPRVQPKPPHGAAENSLSPSSNRVKF
SRSADAPAYQQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR
SEQ ID NO: 55:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDPQLCYILDAI
LFLYGIVLTLLYCRLKIQVRKAAITSYEKSDGVYTGLSTRNQETYETLKHEKPPQKRGRKKLLYIFK
QPFMRPVQTTQEEDGCSCRFPEEEEGGCEL
SEQ ID NO: 56:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRLKIQVRKAAIT
SYEKSDGVYTGLSTRNQETYETLKHEKPPQ
SEQ ID NO: 57:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQIEVMYPPPYLDNEKSNGTIIHVKGKHLCPSPLFPGPSKPFWVLVVVGGVLACYS
LLVTVAFIIFWVRSKRSRLLHSDYMNMTPRRPGPTRKHYQPYAPPRDFAAYRSRVKFSRSADAPAY
QQGQNQLYNELNLGRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 58:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQIYIWAPLAGTCGVLLLSLVITLYCRSKRSRLLHSDYMNMTPRRPGPTRKHYQPY
APPRDFAAYRSRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR
SEQ ID NO: 59:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCRSKRSRLLHSDYMNMTPRRPGPTRKHYQPYAPPRDFAAYRSQRRKYRSNKGE
SPVEPAEPCHYSCPREEEGSTIPIQEDYRKPEPACSPRVKFSRSADAPAYQQGQNQLYNELNLGRREE
YDVLDKRRGRDPEMGGKPRRKNPQEGLYNELQKDKMAEAYSEIGMKGERRRGKGHDGLYQGLS
TATKDTYDALHMQALPPR
SEQ ID NO: 60:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCRSKRSRLLHSDYMNMTPRRPGPTRKHYQPYAPPRDFAAYRSRRDQRLPPDAH
KPPGGGSFRTPIQEEQADAHSTLAKIRVKFSRSADAPAYQQGQNQLYNELNLGRREEYDVLDKRRG
RDPEMGGKPRRKNPQEGLYNELQKDKMAEAYSEIGMKGERRRGKGHDGLYQGLSTATKDTYDAL
HMQALPPR
SEQ ID NO: 61:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRSKRSRLLHSD
YMNMTPRRPGPTRKHYQPYAPPRDFAAYRSRVKFSRSADAPAYQQGQNQLYNELNLGRREEYDV
LDKRRGRDPEMGGKPRRKNPQEGLYNELQKDKMAEAYSEIGMKGERRRGKGHDGLYQGLSTATK
DTYDALHMQALPPR
SEQ ID NO: 62:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQIEVMYPPPYLDNEKSNGTIIHVKGKHLCPSPLFPGPSKPFWVLVVVGGVLACYS
LLVTVAFIIFWVRSKRSRLLHSDYMNMTPRRPGPTRKHYQPYAPPRDFAAYRSKRGRKKLLYIFKQ
PFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDT
YDALHMQALPPR
SEQ ID NO: 63:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQIEVMYPPPYLDNEKSNGTIIHVKGKHLCPSPLFPGPSKPFWVLVVVGGVLACYS
LLVTVAFIIFWVKRGRKKLLYIFKQPFMRPVQTTQEEDGCSCRFPEEEEGGCELRVKFSRSADAPAY
QQGQNQLYNELNLGRREEYDVLDKRRGRDPEMGGKPRRKNPQEGLYNELQKDKMAEAYSEIGM
KGERRRGKGHDGLYQGLSTATKDTYDALHMQALPPR
SEQ ID NO: 64:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCQRRKYRSNKGESPVEPAEPCHYSCPREEEGSTIPIQEDYRKPEPACSPRVKFSRS
ADAPAYQQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
SEQ ID NO: 65:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCRSKRSRLLHSDYMNMTPRRPGPTRKHYQPYAPPRDFAAYRSRVKFSRSADAP
AYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 66:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCKKKYSSSVHDPNGEYMFMRAVNTAKKSRLTDVTLRVKFSRSADAPAYQQGQ
NQLYNELNLGRREEYDVLDKRRGRDPEMGGKPRRKNPQEGLYNELQKDKMAEAYSEIGMKGERR
RGKGHDGLYQGLSTATKDTYDALHMQALPPR
SEQ ID NO: 67:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCRRDQRLPPDAHKPPGGGSFRTPIQEEQADAHSTLAKIRVKFSRSADAPAYQQG
QNQLYNELNLGRREEYDVLDKRRGRDPEMGGKPRRKNPQEGLYNELQKDKMAEAYSEIGMKGER
RRGKGHDGLYQGLSTATKDTYDALHMQALPPR
SEQ ID NO: 68:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCRSKRSRLLHSDYMNMTPRRPGPTRKHYQPYAPPRDFAAYRSKKKYSSSVHDP
NGEYMFMRAVNTAKKSRLTDVTLRVKFSRSADAPAYQQGQNQLYNELNLGRREEYDVLDKRRGR
DPEMGGKPRRKNPQEGLYNELQKDKMAEAYSEIGMKGERRRGKGHDGLYQGLSTATKDTYDALH
MQALPPR
SEQ ID NO: 69:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQIYIWAPLAGTCGVLLLSLVITLYCKRGRKKLLYIFKQPFMRPVQTTQEEDGCSC
RFPEEEEGGCELRVKFSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR
SEQ ID NO: 70:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQIYIWAPLAGTCGVLLLSLVITLYCQRRKYRSNKGESPVEPAEPCHYSCPREEEGS
TIPIQEDYRKPEPACSPRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQALPPR
SEQ ID NO: 71:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQIYIWAPLAGTCGVLLLSLVITLYCKKKYSSSVHDPNGEYMFMRAVNTAKKSRL
TDVTLRVKFSRSADAPAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQALPPR
SEQ ID NO: 72:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQIYIWAPLAGTCGVLLLSLVITLYCRRDQRLPPDAHKPPGGGSFRTPIQEEQADAH
STLAKIRVKFSRSADAPAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQALPPR
SEQ ID NO: 73:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQFACDIYIWAPLAGTCGVLLLSLVITLYCKRGRKKLLYIFKQPFMRPVQTTQEED
GCSCRFPEEEEGGCELRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQALPPR
SEQ ID NO: 74:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQFACDIYIWAPLAGTCGVLLLSLVITLYCRSKRSRLLHSDYMNMTPRRPGPTRKH
YQPYAPPRDFAAYRSRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQALPPR
SEQ ID NO: 75:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDFWVLVVVGG
VLACYSLLVTVAFIIFWVRSKRSRLLHSDYMNMTPRRPGPTRKHYQPYAPPRDFAAYRSRVKFSRS
ADAPAYQQGQNQLYNELNLGRREEYDVLDKRRGRDPEMGGKPRRKNPQEGLYNELQKDKMAEA
YSEIGMKGERRRGKGHDGLYQGLSTATKDTYDALHMQALPPR
SEQ ID NO: 76:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQKSNGTIIHVKGKHLCPSPLFPGPSKPFWVLVVVGGVLACYSLLVTVAFIIFWVRS
KRSRLLHSDYMNMTPRRPGPTRKHYQPYAPPRDFAAYRSRVKFSRSADAPAYQQGQNQLYNELNL
GRREEYDVLDKRRGRDPEMGGKPRRKNPQEGLYNELQKDKMAEAYSEIGMKGERRRGKGHDGL
YQGLSTATKDTYDALHMQALPPR
SEQ ID NO: 77:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQGKHLCPSPLFPGPSKPFWVLVVVGGVLACYSLLVTVAFIIFWVRSKRSRLLHSD
YMNMTPRRPGPTRKHYQPYAPPRDFAAYRSRVKFSRSADAPAYQQGQNQLYNELNLGRREEYDV
LDKRRGRDPEMGGKPRRKNPQEGLYNELQKDKMAEAYSEIGMKGERRRGKGHDGLYQGLSTATK
DTYDALHMQALPPR
SEQ ID NO: 78:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQFWVLVVVGGVLACYSLLVTVAFIIFWVRSKRSRLLHSDYMNMTPRRPGPTRKH
YQPYAPPRDFAAYRSRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKDTYDALHMQALPPR
SEQ ID NO: 79:
MALPVTALLLPLALLLHAARPGMRTEDLPKAVVFLEPQWYRVLEKDSVTLKCQGAYSPEDNSTQWF
HNESLISSQASSYFIDAATVDDSGEYRCQTNLSTLSDPVQLEVHIGWLLLQAPRWVFKEEDPIHLRC
HSWKNTALHKVTYLQNGKGRKYFHHNSDFYIPKATLKDSGSYFCRGLVGSKNVSSETVNITITQGL
AVSTISSFFPPGYQTTTPAPRPPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEEEGGCELRVKFSRSADAP
AYQQGQNQLYNELNLGRREEYDVLDKRRGRDPEMGGKPRRKNPQEGLYNELQKDKMAEAYSEIG
MKGERRRGKGHDGLYQGLSTATKDTYDALHMQALPPR
SEQ ID NO: 80:
MALPVTALLLPLALLLHAARPGMRTEDLPKAVVFLEPQWYRVLEKDSVTLKCQGAYSPEDNSTQWFHNE
SLISSQASSYFIDAATVDDSGEYRCQTNLSTLSDPVQLEVHIGWLLLQAPRWVFKEEDPIHLRCHSWKN
TALHKVTYLQNGKGRKYFHHNSDFYIPKATLKDSGSYFCRGLVGSKNVSSETVNITITQGLAVSTISSFF
PPGYQIEVMYPPPYLDNEKSNGTIIHVKGKHLCPSPLFPGPSKPIYIWAPLAGTCGVLLLSLVITLYCRSK
RSRLLHSDYMNMTPRRPGPTRKHYQPYAPPRDFAAYRSRVKFSRSADAPAYQQGQNQLYNELNLGRR
EEYDVLDKRRGRDPEMGGKPRRKNPQEGLYNELQKDKMAEAYSEIGMKGERRRGKGHDGLYQGLST
ATKDTYDALHMQALPPR

### H. Examples of CAR Polypeptides

Exemplary CAR polypeptides for use with the methods and compositions described herein may be found, for example, in the instant description and figures or as those known in the art. The CAR polypeptides described herein may comprise an extracellular domain comprising a single-chain antibody fragment (scFv) with binding affinity and specificity for an antigen of interest (e.g., those listed in Table 3 above), a transmembrane domain, and a CD3ζ cytoplasmic signaling domain. In some embodiments, the CAR polypeptides may further include one or more co-stimulatory signaling domains, one of which may be a CD28 co-stimulatory signaling domain or a 4-1BB co-stimulatory signaling domain. The CAR polypeptides are configured such that, when expressed on a host cell, the extracellular antigen-binding domain is located extracellularly for binding to a target molecule and the CD3ζ cytoplasmic signaling domain. The co-stimulatory signaling domain may be located in the cytoplasm for triggering activation and/or effector signaling.

In some embodiments, a CAR polypeptide as described herein may comprise, from N-terminus to C-terminus, the extracellular antigen binding domain, the transmembrane domain, the optional one or more co-stimulatory domains (e.g., a CD28 co-stimulatory domain, a 4-1BB co-stimulatory signaling domain, an OX40 co-stimulatory signaling domain, a CD27 co-stimulatory signaling domain, or an ICOS co-stimulatory signaling domain), and the CD3ζ cytoplasmic signaling domain.

Alternatively or in addition, the CAR polypeptides described herein may contain two or more co-stimulatory signaling domains, which may link to each other or be separated by the cytoplasmic signaling domain. The extracellular antigen binding domain, transmembrane domain, optional co-stimulatory signaling domain(s), and cytoplasmic signaling domain in a CAR polypeptide may be linked to each other directly, or via a peptide linker. In some embodiments, any of the CAR polypeptides described herein may comprise a signal sequence at the N-terminus.

Table 5 provides exemplary CAR polypeptides described herein. These exemplary constructs have, from N-terminus to C-terminus in order, the signal sequence, the antigen binding domain (e.g., a scFv fragment targeting an antigen such as a tumor antigen or a pathogenic antigen), the hinge domain, and the transmembrane, while the positions of the optional co-stimulatory domain and the cytoplasmic signaling domain can be switched.

TABLE 5
Exemplary Components of CAR polypeptides.

| | Extracellular | | | | Cyto- |
|---|---|---|---|---|---|
| | domain | | Trans- | Co- | plasmic |
| Signal | (antigen | Hinge | membrane | stimulatory | Signaling |
| Sequence | binding) | domain | domain | domain | domain |
| CD8α | scFv (e.g., | CD8 | CD8 | 4-1BB | CD3ζ |
| | anti-GPC3 | | | | |
| | scFv) | | | | |
| CD8α | scFv (e.g., | CD28 | CD28 | CD28 | CD3ζ |
| | anti-GPC3 | | | | |
| | scFv) | | | | |

Amino acid sequences of the example CAR polypeptides are provided below (signal sequence italicized).

SEQ ID NO: 104:
MALPVTALLLPLALLLHAARPDVVMTQSPLSLPVTPGEPASISCRSSQSLVHSNRNTYLHWYLQKPGQSPQ
LLIYKVSNRFSGVPDRFSGSGSGTDFTLKISRVEAEDVGVYYCSQNTHVPPTFGQGTKLEIKRGGGGSG
GGGSGGGGSQVQLVQSGAEVKKPGASVKVSCKASGYTFTDYEMHWVRQAPGQGLEWMGALDPKTG
DTAYSQKFKGRVTLTADKSTSTAYMELSSLTSEDTAVYYCTRFYSYTYWGQGTLVTVSSTTTPAPRPP
TPAPTIASQPLSLRPEACRPAAGGAVHTRGLDFACDIYIWAPLAGTCGVLLLSLVITLYCKRGRKKLLYI
FKQPFMRPVQTTQEEDGCSCRFPEEEEGGCELRVKFSRSADAPAYQQGQNQLYNELNLGRREEYDVLD
KRRGRDPEMGGKPRRKNPQEGLYNELQKDKMAEAYSEIGMKGERRRGKGHDGLYQGLSTATKDTYD
ALHMQALPPR
SEQ ID NO: 105:
MALPVTALLLPLALLLHAARPDVVMTQSPLSLPVTPGEPASISCRSSQSLVHSNRNTYLHWYLQKPGQSPQ
LLIYKVSNRFSGVPDRFSGSGSGTDFTLKISRVEAEDVGVYYCSQNTHVPPTFGQGTKLEIKRGGGGSG
GGGSGGGGSQVQLVQSGAEVKKPGASVKVSCKASGYTFTDYEMHWVRQAPGQGLEWMGALDPKTG
DTAYSQKFKGRVTLTADKSTSTAYMELSSLTSEDTAVYYCTRFYSYTYWGQGTLVTVSSIEVMYPPPY
LDNEKSNGTIIHVKGKHLCPSPLFPGPSKPFWVLVVVGGVLACYSLLVTVAFIIFWVRSKRSRLLHSDY
MNMTPRRPGPTRKHYQPYAPPRDFAAYRSRVKFSRSADAPAYQQGQNQLYNELNLGRREEYDVLDK
RRGRDPEMGGKPRRKNPQEGLYNELQKDKMAEAYSEIGMKGERRRGKGHDGLYQGLSTATKDTYDA
LHMQALPPR

### III. Hematopoietic Cells Expressing Krebs Cycle Modulating Polypeptides and Optionally Chimeric Receptor Polypeptides

Provided herein are genetically engineered host cells (e.g., hematopoietic cells such as HSCs and immune cells, e.g., T cells or NK cells) expressing one or more of the Krebs cycle modulating polypeptides as described herein. The genetically engineered host cells may further express a chimeric receptor polypeptide (e.g., ACTR-expressing cells, e.g., ACTR Tcells or CAR-expressing cells, e.g., CAR T cells) as also described herein. In some embodiments, the host cells are hematopoietic cells or a progeny thereof. In some embodiments, the hematopoietic cells can be hematopoietic stem cells. In other embodiments, the host cells are immune cells, such as T cells or NK cells. In some embodiments, the immune cells are T cells. In some embodiments, the immune cells are NK cells. In other embodiments, the immune cells can be established cell lines, for example, NK-92 cells.

In some embodiments, the genetically engineered hematopoietic cells such as HSCs or immune cells (e.g., T cells or NK cells) may co-express any of the CAR constructs such as those disclosed herein with any of the Krebs cycle modulating agents, such as a Krebs cycle modulating polypeptide (e.g., GOT1 or GOT2). In some embodiments, the CAR construct may comprise a co-stimulatory domain from 4-1BB or CD28 and the Krebs cycle modulating polypeptide is GOT1 or GOT2. The CAR construct may further comprise a hinge and transmembrane domain from CD8 or CD28.

In other embodiments, the genetically engineered hematopoietic cells such as HSCs or immune cells (e.g., T cells or NK cells) may co-express any of the ACTR constructs such as those disclosed herein with any of the Krebs cycle modulating agents, such as a Krebs cycle modulating polypeptide (e.g., GOT1 or GOT2). In some embodiments, the ACTR construct may comprise a co-stimulatory domain from 4-1BB or CD28 and the Krebs cycle modulating polypeptide is GOT1 or GOT2. The ACTR constructs may further comprise a hinge and transmembrane domain from CD8 or CD28.

Alternatively, the genetically engineered host cells disclosed herein may not express any chimeric receptor polypeptides. In some embodiments, the genetically engineered immune cells, which may overly express one or more Krebs cycle modulating polypeptides as disclosed herein, may be derived from tumor-infiltrating lymphocytes (TILs). Overexpression of the Krebs cycle modulating polypeptides may enhance the anti-tumor activity or the TILs in tumor microenvironment. Alternatively or in addition, the genetically engineered immune cells may be T cells, which may further have genetically engineered T cell receptors. The TILs and/or genetically modified TCRs may target peptide-MHC complex, in which the peptide may be derived from a pathogen, a tumor antigen, or an auto-antigen. Some examples are provided in Table 6 below.

Any of the CAR constructs disclosed herein or an antibody to be co-used with ACTR T cells may also target any of the peptide in such peptide/MHC complex.

TABLE 6
Exemplary Peptide-MHC Targets

| Targets | Indications |
|---|---|
| NY-ESO-1 | Sarcoma, MM |
| MAGE-A10 | NSCLC, Bladder, HNSCC |
| MAGE-A4 | Sarcomas, others |
| PMEL | Melanoma |
| WT-1 | Ovarian |
| AFP | HCC |
| HPV-16 E6 | Cervical |
| HPV-16 E7 | Cervical |

In some embodiments, the host cells are immune cells, such as T cells or NK cells. In some embodiments, the immune cells are T cells. For example, the T cells can be CD4+ helper cells or CD8+ cytotoxic cells, or a combination thereof. Alternatively or in addition, the T cells can be suppressive T cells such as Treg cells. In some embodiments, the immune cells are NK cells. In other embodiments, the immune cells can be established cell lines, for example, NK-92 cells. In some examples, the immune cells can be a mixture of different types of T cells and/or NK cells as known in the art. For example, the immune cells can be a population of immune cells isolated from a suitable donor (e.g., a human patient). Sec disclosures below.

In some instances, the Krebs cycle modulating polypeptide to be introduced into the host cells is identical to an endogenous protein of the host cell. Introducing additional copies of the coding sequences of the Krebs cycle modulating polypeptide into the host cell would enhance the expression level of the polypeptide (i.e., overly expressed) as relative to the native counterpart. In some instances, the Krebs cycle modulating polypeptide to be introduced into the host cells is heterologous to the host cell, i.e., does not exist or is not expressed in the host cell. Such a heterologous Krebs cycle modulating polypeptide may be a naturally-occurring protein not expressed in the host cell in nature (e.g., from a different species). Alternatively, the heterologous Krebs cycle modulating polypeptide may be a variant of a native protein, such as those described herein. In some examples, the exogenous (i.e., not native to the host cells) copy of the coding nucleic acid may exist extrachromosomally. In other examples, the exogenous copy of the coding sequence may be integrated into the chromosome of the host cell, and may be located at a site that is different from the native loci of the endogenous gene.

Such genetically engineered host cells have the capacity to have an enhanced rate of glycolysis and may, for example, have an enhanced capacity of taking glucose from the environment. Thus, these genetically engineered host cells may exhibit better growth and/or bioactivities under low glucose, low amino acid, low pH, and/or hypoxic conditions, for example in a tumor microenvironment. The genetically engineered cells, when expressing a chimeric receptor polypeptide as disclosed herein, can recognize and inhibit target cells, cither directly (e.g., by CAR-expressing immune cells) or via an Fc-containing therapeutic agents such as an anti-tumor antibodies (e.g., by ACTR-expressing immune cells). Given their expected high proliferation rate, bioactivity, and/or survival rate in low glucose, low amino acid, low pH, and/or hypoxic environments (e.g., in a tumor microenvironment), the genetically engineered cells such as T cell and NK cells would be expected to have higher therapeutic efficacy relative to chimeric receptor polypeptide T cells that do not express or express a lower level or less active form of the Krebs cycle modulating polypeptide.

The population of immune cells can be obtained from any source, such as peripheral blood mononuclear cells (PBMCs), bone marrow, or tissues such as spleen, lymph node, thymus, stem cells, or tumor tissue. Alternatively, the immune cell population may be derived from stem cells, for example, hematopoietic stem cells and induced pluripotent stem cells (iPSCs). A source suitable for obtaining the type of host cells desired would be evident to one of skill in the art. In some embodiments, the population of immune cells is derived from PBMCs, which may be obtained from a patient (e.g., a human patient) who needs the treatment described herein. The type of host cells desired (e.g., T cells, NK cells, or T cells and NK cells) may be expanded within the population of cells obtained by co-incubating the cells with stimulatory molecules. As a non-limiting example, anti-CD3 and anti-CD28 antibodies may be used for expansion of T cells.

To construct the immune cells that express any of the Krebs cycle modulating polypeptides and optionally the chimeric receptor polypeptide described herein, expression vectors for stable or transient expression of the Krebs cycle modulating polypeptides and/or the chimeric receptor polypeptide may be created via conventional methods as described herein and introduced into immune host cells. For example, nucleic acids encoding the Krebs cycle modulating polypeptides and/or the chimeric receptor polypeptides may be cloned into one or two suitable expression vectors, such as a viral vector or a non-viral vector in operable linkage to a suitable promoter. In some instances, each of the coding sequences for the chimeric receptor polypeptide and the Krebs cycle modulating polypeptide are on two separate nucleic acid molecules and can be cloned into two separate vectors, which may be introduced into suitable host cells simultaneously or sequentially. Alternatively, the coding sequences for the chimeric receptor polypeptide and the Krebs cycle modulating polypeptide are on one nucleic acid molecule and can be cloned into one vector. The coding sequences of the chimeric receptor polypeptide and the Krebs cycle modulating polypeptide may be in operable linkage to two distinct promoters such that the expression of the two polypeptides is controlled by different promoters. Alternatively, the coding sequences of the chimeric receptor polypeptide and the Krebs cycle modulating polypeptide may be in operably linkage to one promoter such that the expression of the two polypeptides is controlled by a single promoter. Suitable sequences may be inserted between the coding sequences of the two polypeptides so that two separate polypeptides can be translated from a single mRNA molecule. Such sequences, for example, IRES or ribosomal skipping site, are well known in the art. Additional descriptions are provided below.

The nucleic acids and the vector(s) may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the Krebs cycle modulating polypeptides and/or the chimeric receptor polypeptides. The synthetic linkers may contain nucleic acid sequences that correspond to a particular restriction site in the vector. The selection of expression vectors/plasmids/viral vectors would depend on the type of host cells for expression of the Krebs cycle modulating polypeptides and/or the chimeric receptor polypeptides, but should be suitable for integration and replication in eukaryotic cells.

A variety of promoters can be used for expression of the Krebs cycle modulating polypeptides and/or the chimeric receptor polypeptides described herein, including, without limitation, cytomegalovirus (CMV) intermediate early promoter, a viral LTR such as the Rous sarcoma virus LTR, HIV-LTR, HTLV-1 LTR, the simian virus 40 (SV40) early promoter, the human EF1-alpha promoter, or herpes simplex tk virus promoter. Additional promoters for expression of the Krebs cycle modulating polypeptides and/or the chimeric receptor polypeptides include any constitutively active promoter in an immune cell. Alternatively, any regulatable promoter may be used, such that its expression can be modulated within an immune cell.

Additionally, the vector may contain, for example, some or all of the following: a selectable marker gene, such as the neomycin gene or the kanamycin gene for selection of stable or transient transfectants in host cells; enhancer/promoter sequences from the immediate early gene of human CMV for high levels of transcription; intron sequences from the human EF1-alpha gene, transcription termination and RNA processing signals from SV40 for mRNA stability; SV40 polyomavirus origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase or an inducible caspase such as iCasp9), and reporter gene for assessing expression of the Krebs cycle modulating polypeptides and/or the chimeric receptor polypeptide.

In one specific embodiment, such vectors also include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine deaminase, purine nucleoside phosphorylase, nitroreductase, and caspases such as caspase 8.

Suitable vectors and methods for producing vectors containing transgenes are well known and available in the art. Examples of the preparation of vectors for expression of Krebs cycle modulating polypeptides and/or chimeric receptor polypeptides can be found, for example, in US2014/0106449, herein incorporated in its entirety by reference.

Any of the vectors comprising a nucleic acid sequence that encodes a Krebs cycle modulating polypeptide and/or a chimeric receptor polypeptide described herein is also within the scope of the present disclosure. Such a vector, or the sequence encoding a Krebs cycle modulating polypeptide and/or a chimeric receptor polypeptide contained therein, may be delivered into host cells such as host immune cells by any suitable method. Methods of delivering vectors to immune cells are well known in the art and may include DNA electroporation, RNA electroporation, transfection using reagents such as liposomes, or viral transduction (e.g., retroviral transduction such as lentiviral transduction).

In some embodiments, the vectors for expression of the Krebs cycle modulating polypeptides and/or the chimeric receptor polypeptides are delivered to host cells by viral transduction (e.g., retroviral transduction such as lentiviral or gamma-retroviral transduction). Exemplary viral methods for delivery include, but are not limited to, recombinant retroviruses (see, e.g., PCT Publication Nos. WO 90/07936; WO 94/03622; WO 93/25698; WO 93/25234; WO 93/11230; WO 93/10218; and WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191; WO 94/28938; WO 95/11984; and WO 95/00655). In some embodiments, the vectors for expression of the Krebs cycle modulating polypeptides and/or the chimeric receptor polypeptides are retroviruses. In some embodiments, the vectors for expression of the Krebs cycle modulating polypeptides and/or the chimeric receptor polypeptides are lentiviruses.

Examples of references describing retroviral transduction include Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993). International Patent Publication No. WO 95/07358 describes high efficiency transduction of primary B lymphocytes. See also WO 2016/040441A1, which is incorporated by reference herein for the purpose and subject matter referenced herein.

In examples in which the vectors encoding Krebs cycle modulating polypeptides and/or chimeric receptor polypeptides are introduced to the host cells using a viral vector, viral particles that are capable of infecting the immune cells and carry the vector may be produced by any method known in the art and can be found, for example in PCT Application No. WO 1991/002805A2, WO 1998/009271 A1, and U.S. Pat. No. 6,194,191. The viral particles are harvested from the cell culture supernatant and may be isolated and/or purified prior to contacting the viral particles with the immune cells.

In some embodiments, RNA molecules encoding any of the Krebs cycle modulating polypeptides and/or the chimeric receptor polypeptides as described herein may be prepared by a conventional method (e.g., in vitro transcription) and then introduced into suitable host cells, e.g., those described herein, via known methods, e.g., Rabinovich et al., Human Gene Therapy 17:1027-1035.

In some instances, the nucleic acid encoding a Krebs cycle modulating polypeptide and the nucleic acid encoding a suitable chimeric receptor polypeptide may be cloned into separate expression vectors, which may be introduced into suitable host cells concurrently or sequentially. For example, an expression vector (or an RNA molecule) for expressing the Krebs cycle modulating polypeptide may be introduced into host cells first and transfected host cells expressing the Krebs cycle modulating polypeptide may be isolated and cultured in vitro. An expression vector (or an RNA molecule) for expressing a suitable chimeric receptor polypeptide can then introduced into the host cells that express the Krebs cycle modulating polypeptide and transfected cells expressing both polypeptides can be isolated. In another example, expression vectors (or RNA molecules) each for expressing the Krebs cycle modulating polypeptide and the chimeric receptor polypeptide can be introduced into host cells simultaneously and transfected host cells expressing both polypeptides can be isolated via routine methodology.

In other instances, the nucleic acid encoding the Krebs cycle modulating polypeptide and the nucleic acid encoding the chimeric receptor polypeptide may be cloned into the same expression vector. Polynucleotides (including vectors in which such polynucleotides are operably linked to at least one regulatory element) for expression of the chimeric receptor polypeptide and Krebs cycle modulating polypeptide are also within the scope of the present disclosure. Non-limiting examples of useful vectors of the disclosure include viral vectors such as, e.g., retroviral vectors including gamma retroviral vectors and lentiviral vectors, and adeno-associated virus vectors (AAV vectors).

In some instances, the nucleic acid(s) encoding the Krebs cycle modulating polypeptide and/or the chimeric receptor polypeptide may be delivered into host cells via transposon. In some instances, the encoding nucleic acid(s) may be delivered into host cells via gene editing, for example, by CRISPR, TALEN, zinc-finger nuclease (ZFN), or meganucleases.

In some instances, the nucleic acid described herein may comprise two coding sequences, one encoding a chimeric receptor polypeptide as described herein, and the other encoding a polypeptide capable of modulating the Krebs cycle (i.e., a Krebs cycle modulating polypeptide). The nucleic acid comprising the two coding sequences described herein may be configured such that the polypeptides encoded by the two coding sequences can be expressed as independent (and physically separate) polypeptides. To achieve this goal, the nucleic acid described herein may contain a third nucleotide sequence located between the first and second coding sequences. This third nucleotide sequence may, for example, encode a ribosomal skipping site. A ribosomal skipping site is a sequence that impairs normal peptide bond formation. This mechanism results in the translation of additional open reading frames from one messenger RNA. This third nucleotide sequence may, for example, encode a P2A, T2A, or F2A peptide (see, for example, Kim et al., PLOS One. 2011; 6 (4): e18556). As a non-limiting example, an exemplary P2A peptide may have the amino acid sequence of ATNFSLLKQAGDVEENPGP SEQ ID NO.: 106.

In another embodiment, the third nucleotide sequence may encode an internal ribosome entry site (IRES). An IRES is an RNA element that allows translation initiation in an end-independent manner, also permitting the translation of additional open reading frames from one messenger RNA. Alternatively, the third nucleotide sequence may encode a second promoter controlling the expression of the second polypeptide. The third nucleotide sequence may also encode more than one ribosomal skipping sequence, IRES sequence, additional promoter sequence, or a combination thereof.

The nucleic acid may also include additional coding sequences (including, but not limited to, fourth and fifth coding sequences) and may be configured such that the polypeptides encoded by the additional coding sequences are expressed as further independent and physically separate polypeptides. To this end, the additional coding sequences may be separated from other coding sequences by one or more nucleotide sequences encoding one or more ribosomal skipping sequences, IRES sequences, or additional promoter sequences.

In some examples, the nucleic acid (e.g., an expression vector or an RNA molecule as described herein) may comprise coding sequences for both the Krebs cycle modulating polypeptide (e.g., those described herein) and a suitable chimeric receptor polypeptide, the two coding sequences, in any order, being separated by a third nucleotide sequence coding for a P2A peptide (e.g., ATNFSLLKQAGDVEENPGP; SEQ ID NO: 106). As a result, two separate polypeptides, the Krebs cycle modulating polypeptide and the chimeric receptor, can be produced from such a nucleic acid, wherein the P2A portion ATNFSLLKQAGDVEENPG (SEQ ID NO: 107) is linked to the upstream polypeptide (encoded by the upstream coding sequence) and residue P from the P2A peptide is linked to the downstream polypeptide (encoded by the downstream coding sequence). In some examples, the chimeric receptor polypeptide is the upstream one and the Krebs cycle modulating polypeptide is the downstream one. In other examples, the Krebs cycle modulating polypeptide is the upstream one and the chimeric receptor polypeptide is the downstream one.

In some examples, the nucleic acid (e.g., an expression vector or an RNA molecule as described herein) may comprise coding sequences for both the Krebs cycle modulating polypeptide (e.g., those described herein) and a suitable ACTR polypeptide, the two coding sequences, in any order, being separated by a third nucleotide sequence coding for a P2A peptide (e.g., ATNFSLLKQAGDVEENPGP; SEQ ID NO:106). As a result, two separate polypeptides, the Krebs cycle modulating polypeptide and the ACTR) can be produced from such a nucleic acid, wherein the P2A portion ATNFSLLKQAGDVEENPG (SEQ ID NO: 107) is linked to the upstream polypeptide (encoded by the upstream coding sequence) and residue P from the P2A peptide is linked to the downstream polypeptide (encoded by the downstream coding sequence). In some examples, the ACTR polypeptide is the upstream one and the Krebs cycle modulating polypeptide is the downstream one. In other examples, the Krebs cycle modulating polypeptide is the upstream one and the ACTR polypeptide is the downstream one.

In some examples, the nucleic acid described above may further encode a linker (e.g., a GSG linker) between two segments of the encoded sequences, for example, between the upstream polypeptide and the P2A peptide.

In specific examples, the nucleic acid described herein is configured such that it expresses two separate polypeptides in the host cell to which the nucleic acid is transfected: (i) the first polypeptide that contains, from the N-terminus to the C-terminus, a suitable CAR (e.g., SEQ ID NO: 104 or SEQ ID NO: 105), a peptide linker (e.g., the GSG linker), and the ATNFSLLKQAGDVEENPG (SEQ ID NO:107) segment derived from the P2A peptide; and (ii) a second polypeptide that contains, from the N-terminus to the C-terminus, the P residue derived from the P2A peptide and the Krebs cycle modulating polypeptide (e.g., any of SEQ ID NOs: 81-92).

In specific examples, the nucleic acid described herein is configured such that it expresses two separate polypeptides in the host cell to which the nucleic acid is transfected: (i) the first polypeptide that contains, from the N-terminus to the C-terminus, a suitable ACTR (e.g., any of SEQ ID NOs: 1-80 described herein, for example, SEQ ID NO:1 or SEQ ID NO: 57), a peptide linker (e.g., the GSG linker), and the ATNFSLLKQAGDVEENPG (SEQ ID NO: 107) segment derived from the P2A peptide; and (ii) a second polypeptide that contains, from the N-terminus to the C-terminus, the P residue derived from the P2A peptide and the Krebs cycle modulating polypeptide (e.g., any of SEQ ID NOs: 81-92).

In some instances, additional polypeptides of interest may also be introduced into the host immune cells.

Following introduction into the host cells a vector encoding any of the Krebs cycle modulating polypeptides and/or the chimeric receptor polypeptides provided herein, or the nucleic acid encoding the chimeric receptor polypeptide and/or Krebs cycle modulating polypeptide (e.g., an RNA molecule), the cells may be cultured under conditions that allow for expression of the Krebs cycle modulating polypeptides and/or the chimeric receptor polypeptide. In examples in which the nucleic acid encoding the Krebs cycle modulating polypeptides and/or the chimeric receptor polypeptide is regulated by a regulatable promoter, the host cells may be cultured in conditions wherein the regulatable promoter is activated. In some embodiments, the promoter is an inducible promoter and the immune cells are cultured in the presence of the inducing molecule or in conditions in which the inducing molecule is produced. Determining whether the Krebs cycle modulating polypeptide and/or the chimeric receptor polypeptide is expressed will be evident to one of skill in the art and may be assessed by any known method, for example, detection of the Krebs cycle modulating polypeptide and/or the chimeric receptor polypeptide-encoding mRNA by quantitative reverse transcriptase PCR (qRT-PCR) or detection of the Krebs cycle modulating polypeptide and/or the chimeric receptor polypeptide protein by methods including Western blotting, fluorescence microscopy, and flow cytometry.

Alternatively, expression of the chimeric receptor polypeptide may take place in vivo after the immune cells are administered to a subject. As used herein, the term “subject” refers to any mammal such as a human, monkey, mouse, rabbit, or domestic mammal. For example, the subject may be a primate. In a preferred embodiment, the subject is human.

Alternatively, expression of a Krebs cycle modulating polypeptide and/or a chimeric receptor polypeptide in any of the immune cells disclosed herein can be achieved by introducing RNA molecules encoding the Krebs cycle modulating polypeptides and/or the chimeric receptor polypeptides. Such RNA molecules can be prepared by in vitro transcription or by chemical synthesis. The RNA molecules can then be introduced into suitable host cells such as immune cells (e.g., T cells, NK cells, or both T cells and NK cells) by, e.g., electroporation. For example, RNA molecules can be synthesized and introduced into host immune cells following the methods described in Rabinovich et al., Human Gene Therapy, 17:1027-1035 and WO 2013/040557.

In certain embodiments, a vector(s) or RNA molecule(s) comprising the Krebs cycle modulating polypeptide and/or the chimeric receptor polypeptide may be introduced to the host cells or immune cells in vivo. As a non-limiting example, this may be accomplished by administering a vector or RNA molecule encoding one or more Krebs cycle modulating polypeptides and/or one or more chimeric receptor polypeptides described herein directly to the subject (e.g., through intravenous administration), producing host cells comprising Krebs cycle modulating polypeptides and/or chimeric receptor polypeptides in vivo.

Methods for preparing host cells expressing any of the Krebs cycle modulating polypeptides and/or the chimeric receptor polypeptides described herein may also comprise activating the host cells ex vivo. Activating a host cell means stimulating a host cell into an activated state in which the cell may be able to perform effector functions. Methods of activating a host cell will depend on the type of host cell used for expression of the Krebs cycle modulating polypeptides and/or chimeric receptor polypeptides. For example, T cells may be activated ex vivo in the presence of one or more molecules including, but not limited to: an anti-CD3 antibody, an anti-CD28 antibody, IL-2, phytohemoagglutinin, engineered artificial stimulatory cells or particles, or a combination thereof. The engineered artificial stimulatory cells may be artificial antigen-presenting cells as known in the art. Sec, e.g., Neal et al., J. Immunol. Res. Ther. 2017, 2 (1): 68-79 and Turtle et al., Cancer J. 2010, 16 (4): 374-381, the relevant disclosures of each of which are hereby incorporated by reference for the purpose and subject matter referenced herein.

In other examples, NK cells may be activated ex vivo in the presence of one or more molecules such as a 4-1BB ligand, an anti-4-1BB antibody, IL-15, an anti-IL-15 receptor antibody, IL-2, IL12, IL-21, K562 cells, and/or engineered artificial stimulatory cells or particles. In some embodiments, the host cells expressing any of the Krebs cycle modulating polypeptides and/or the chimeric receptor polypeptides (ACTR-/CAR- and/or Krebs cycle modulating polypeptide-expressing cells) described herein are activated ex vivo prior to administration to a subject. Determining whether a host cell is activated will be evident to one of skill in the art and may include assessing expression of one or more cell surface markers associated with cell activation, expression or secretion of cytokines, and cell morphology.

Methods for preparing host cells expressing any of the Krebs cycle modulating polypeptides and/or the chimeric receptor polypeptides described herein may comprise expanding the host cells ex vivo. Expanding host cells may involve any method that results in an increase in the number of cells expressing Krebs cycle modulating polypeptides and/or chimeric receptor polypeptides, for example, allowing the host cells to proliferate or stimulating the host cells to proliferate. Methods for stimulating expansion of host cells will depend on the type of host cell used for expression of the Krebs cycle modulating polypeptides and/or the chimeric receptor polypeptides and will be evident to one of skill in the art. In some embodiments, the host cells expressing any of the Krebs cycle modulating polypeptides and/or the chimeric receptor polypeptides described herein are expanded ex vivo prior to administration to a subject.

In some embodiments, the host cells expressing the Krebs cycle modulating polypeptides and/or the chimeric receptor polypeptides are expanded and activated ex vivo prior to administration of the cells to the subject. Host cell activation and expansion may be used to allow integration of a viral vector into the genome and expression of the gene encoding a Krebs cycle modulating polypeptide and/or a chimeric receptor polypeptide as described herein. If mRNA electroporation is used, no activation and/or expansion may be required, although electroporation may be more effective when performed on activated cells. In some instances, a Krebs cycle modulating polypeptide and/or a chimeric receptor polypeptide is transiently expressed in a suitable host cell (e.g., for 3-5 days). Transient expression may be advantageous if there is a potential toxicity and should be helpful in initial phases of clinical testing for possible side effects.

Any of the host cells expressing the Krebs cycle modulating polypeptides and/or the chimeric receptor polypeptides may be mixed with a pharmaceutically acceptable carrier to form a pharmaceutical composition, which is also within the scope of the present disclosure.

The phrase “pharmaceutically acceptable”, as used in connection with compositions of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. Sec, e.g. Remington: The Science and Practice of Pharmacy 20th Ed. (2000) Lippincott Williams and Wilkins, Ed. K. E. Hoover.

The pharmaceutical compositions of the disclosure may also contain one or more additional active compounds as necessary for the particular indication being treated, preferably those with complementary activities that do not adversely affect each other. Non-limiting examples of possible additional active compounds include, e.g., IL-2 as well as various agents known in the field and listed in the discussion of combination treatments, below.

### IV. Immunotherapy Using the Genetically Engineered Hematopoietic Cells Described Herein

The genetically-engineered hematopoietic cells (e.g., hematopoietic stem cells, immune cells, such as NK cells or T cells) disclosed herein may be used in immunotherapy against various disorders, for example, cancer, infectious diseases, and autoimmune diseases.

### (a) Combined Immunotherapy of Genetically Engineered Hematopoietic Cells Expressing ACTR Polypeptides and Fc-Containing Therapeutic Agents

The exemplary ACTR polypeptides of the present disclosure confer antibody-dependent cell cytotoxicity (ADCC) capacity to T lymphocytes and enhance ADCC in NK cells. When the receptor is engaged by an antibody bound to cells, it triggers T-cell activation, sustained proliferation and specific cytotoxicity against the bound cells.

The degree of affinity of CD16 for the Fc portion of Ig is a critical determinant of ADCC and thus to clinical responses to antibody immunotherapy. The CD16 with the V158 polymorphism which has a higher binding affinity for Ig and mediates superior ADCC relative to CD16 with the F158 polymorphism was selected as an example. Although the F158 receptor has lower potency than the V158 receptor in induction of T cell proliferation and ADCC, the F158 receptor may have lower in vivo toxicity than the V158 receptor making it useful in some clinical contexts.

The Krebs cycle modulating polypeptides to be co-expressed with an ACTR polypeptides in immune cells would facilitate cell-based immune therapy such as T-cell therapy or NK-cell therapy by allowing the cells to grow and/or function effectively in a low glucose, low amino acid, low pH, and/or hypoxic environment. Antibody-directed cytotoxicity could be stopped whenever required by simple withdrawal of antibody administration. Clinical safety can be further enhanced by using mRNA electroporation to express the Krebs cycle modulating polypeptides and/or the ACTR polypeptides transiently, to limit any potential autoimmune reactivity.

Thus, in one embodiment, the disclosure provides a method for enhancing efficacy of an antibody-based immunotherapy of a cancer in a subject in need thereof, which subject is being treated with an Fc-containing therapeutic agent such as a therapeutic antibody, which can bind to antigen-expressing cells. The Fc-containing therapeutic agent contains an Fc portion, for example, a human or humanized Fc portion, which can be recognized and bound by the Fc-binding portion (e.g., the extracellular domain of human CD16A) of the ACTR expressed on the engineered immune cells.

The methods described herein may comprise introducing into the subject a therapeutically effective amount an antibody and a therapeutically effective amount of the genetically engineered host cells such as hematopoietic cells, for example, immune cells (e.g., T lymphocytes or NK cells), which co-express a Krebs cycle modulating polypeptide and an ACTR polypeptide of the disclosure. The subject (e.g., a human patient such as a human cancer patient) has been treated or is being treating with an Fc-containing therapeutic agent specific to a target antigen. A target antigen may be any molecule that is associated with a disease or condition, including, but are not limited to, tumor antigens, pathogenic antigens (e.g., bacterial or viral), or antigens present on diseased cells, such as those described herein.

In the context of the present disclosure insofar as it relates to any of the disease conditions recited herein, the terms “treat”, “treatment”, and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. For example, in connection with cancer the term “treat” may mean eliminate or reduce a patient's tumor burden, or prevent, delay or inhibit metastasis, etc.

As used herein the term “therapeutically effective” applied to dose or amount refers to that quantity of a compound or pharmaceutical composition that is sufficient to result in a desired activity upon administration to a subject in need thereof. Note that when a combination of active ingredients is administered (e.g., a first pharmaceutical composition comprising an antibody, and a second pharmaceutical composition comprising a population of T lymphocytes or NK cells that express a Krebs cycle modulating polypeptide and/or an antibody-coupled T-cell receptor (ACTR) construct), the effective amount of the combination may or may not include amounts of each ingredient that would have been effective if administered individually. Within the context of the present disclosure, the term “therapeutically effective” refers to that quantity of a compound or pharmaceutical composition that is sufficient to delay the manifestation, arrest the progression, relieve or alleviate at least one symptom of a disorder treated by the methods of the present disclosure.

Host cells (e.g., hematopoietic cells, for example, immune cells such as T cells and NK cells) expressing Krebs cycle modulating polypeptides and ACTR polypeptides described herein are useful for enhancing ADCC in a subject and/or for enhancing the efficacy of an antibody-based immunotherapy and/or for enhancing growth and/or proliferation of immune cells in a low-glucose environment. In some embodiments, the subject is a mammal, such as a human, monkey, mouse, rabbit, or domestic mammal. In some embodiments, the subject is a human. In some embodiments, the subject is a human cancer patient. In some embodiments, the subject has been treated or is being treated with any of the therapeutic antibodies described herein.

To practice the method described herein, an effective amount of the host cells, for example, immune cells (e.g., NK cells and/or T lymphocytes) expressing any of the Krebs cycle modulating polypeptides and the ACTR polypeptides described herein and an effective amount of an antibody, or compositions thereof may be administered to a subject in need of the treatment via a suitable route, such as intravenous administration. As used herein, an effective amount refers to the amount of the respective agent (e.g., the NK cells and/or T lymphocytes expressing Krebs cycle modulating polypeptides, ACTR polypeptides, antibodies, or compositions thereof) that upon administration confers a therapeutic effect on the subject. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender, sex, and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human. In some embodiments, the subject in need of treatment is a human cancer patient. In some embodiments, the subject in need of treatment suffers from one or more pathogenic infections (e.g., viral, bacterial, and/or fungal infections).

The methods of the disclosure may be used for treatment of any cancer or any pathogen. Specific non-limiting examples of cancers which can be treated by the methods of the disclosure include, for example, lymphoma, breast cancer, gastric cancer, neuroblastoma, osteosarcoma, lung cancer, skin cancer, prostate cancer, colorectal cancer, renal cell carcinoma, ovarian cancer, rhabdomyosarcoma, leukemia, mesothelioma, pancreatic cancer, head and neck cancer, retinoblastoma, glioma, glioblastoma, thyroid cancer, hepatocellular cancer, esophageal cancer, and cervical cancer. In certain embodiments, the cancer may be a solid tumor.

The methods of this disclosure may also be used for treating infectious diseases, which may be caused by bacterial infection, viral infection, or fungus infection. In such instances, the genetically engineered immune cells can be co-used with an Fc-containing therapeutic agent (e.g., an antibody) that targets a pathogenic antigen (e.g., an antigen associated with the bacterium, virus, or fungus that causes the infection). Specific non-limiting examples of pathogenic antigens include, but are not limited to, bacterial, viral, and/or fungal antigens. Some examples are provided below: influenza virus neuraminidase, hemagglutinin, or M2 protein, human respiratory syncytial virus (RSV) F glycoprotein or G glycoprotein, herpes simplex virus glycoprotein gB, gC, gD, or gE, Chlamydia MOMP or PorB protein, Dengue virus core protein, matrix protein, or glycoprotein E, measles virus hemagglutinin, herpes simplex virus type 2 glycoprotein gB, poliovirus I VP1, envelope glycoproteins of HIV 1, hepatitis B core antigen or surface antigen, diptheria toxin, Streptococcus 24M epitope, Gonococcal pilin, pseudorabies virus g50 (gpD), pseudorabies virus II (gpB), pseudorabies virus III (gpC), pseudorabies virus glycoprotein H, pseudorabies virus glycoprotein E, transmissible gastroenteritis glycoprotein 195, transmissible gastroenteritis matrix protein, or human hepatitis C virus glycoprotein El or E2.

In some embodiments, the immune cells are administered to a subject in an amount effective in enhancing ADCC activity by least 20% and/or by at least 2-fold, e.g., enhancing ADCC by 50%, 80%, 100%, 2-fold, 5-fold, 10-fold, 20-fold, 50-fold, 100-fold, or more.

The immune cells are co-administered with an Fc-containing therapeutic agent such as a therapeutic antibody in order to target cells expressing the antigen to which the Fc-containing therapeutic agent binds. In some embodiments, more than one Fc-containing therapeutic agents, such as more than one antibodies can be co-used with the immune cells. Antibody-based immunotherapy may be used to treat, alleviate, or reduce the symptoms of any disease or disorder for which the immunotherapy is considered useful in a subject.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof which comprise an Fc region, mutants thereof, fusion proteins comprising an antibody portion, humanized antibodies, chimeric antibodies, diabodies, single domain antibodies (e.g., nanobodies), linear antibodies, multispecific antibodies (e.g., bispecific antibodies) and any other modified configuration of the immunoglobulin molecule that comprises an antigen recognition site of the required specificity and an Fc region, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. The antibody for use in the present disclosure contains an Fc region recognizable by the co-used ACTR- and/or Krebs cycle modulating enhancing polypeptide-expressing immune cells. The Fc region may be a human or humanized Fc region.

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In one example, the antibody used in the methods described herein is a humanized antibody. Humanized antibodies refer to forms of non-human (e.g. murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. The antibodies used herein may be glycosylated (e.g., fucosylated) or afucoslylated. Other forms of humanized antibodies have one or more CDRs (one, two, three, four, five, six) which are altered with respect to the original antibody, which are also termed one or more CDRs “derived from” one or more CDRs from the original antibody. Humanized antibodies may also involve affinity maturation.

In another example, the antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as a human. In some embodiments, amino acid modifications can be made in the variable region and/or the constant region.

The hematopoietic cells, for example, immune cells (e.g., T lymphocytes and/or NK cells) or HSCs expressing any of the Krebs cycle modulating polypeptides and/or the ACTR polypeptides disclosed herein may be administered to a subject who has been treated or is being treated with an Fc-containing antibody. For example, the immune cells may be administered to a human subject simultaneously with an antibody. Alternatively, the immune cells may be administered to a human subject during the course of an antibody-based immunotherapy. In some examples, the immune cells and an antibody can be administered to a human subject at least 4 hours apart, e.g., at least 12 hours apart, at least 1 day apart, at least 3 days apart, at least one week apart, at least two weeks apart, or at least one month apart.

In some embodiments, the antibodies described herein specifically bind to the corresponding target antigen or an epitope thereof. An antibody that “specifically binds” to an antigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to an antigen or an antigenic epitope therein is an antibody that binds this target antigen with greater affinity, avidity, more readily, and/or with greater duration than it binds to other antigens or other epitopes in the same antigen. It is also understood with this definition that, for example, an antibody that specifically binds to a first target antigen may or may not specifically or preferentially bind to a second target antigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen.

In some embodiments, an antibody as described herein has a suitable binding affinity for the target antigen (e.g., any one of the targets described herein) or antigenic epitopes thereof. The antibodies for use in the immune therapy methods described herein may bind to (e.g., specifically bind to) a target antigen of interest, or a specific region or an antigenic epitope therein. Table 3 above lists exemplary target antigens of interest and exemplary antibodies specific to such.

### (b) Immunotherapy of Genetically Engineered Hematopoietic Cells Expressing CAR Polypeptides

The genetically engineered hematopoietic cells (e.g., hematopoietic stem cells, immune cells, such as T cells or natural killer cells) described herein, co-expressing a Krebs cycle modulating polypeptide and a CAR polypeptide can be used in immune therapy such as T-cell therapy or NK-cell therapy for inhibiting diseased cells expressing an antigen to which the CAR polypeptide targets, directly or indirectly (e.g., via a therapeutic agent conjugated to a tag to which the CAR polypeptide binds). The Krebs cycle modulating polypeptide co-expressed with a CAR polypeptide in immune cells would facilitate the cell-based immune therapy by allowing the cells to grow and/or function effectively in a low glucose, low amino acid, low pH, and/or a hypoxic environment, for example, in a tumor microenvironment. Clinical safety may be further enhanced by using mRNA electroporation to express the Krebs cycle modulating polypeptides and/or the CAR polypeptides transiently, to limit any potential non-tumor specific reactivity.

The methods described herein may comprise introducing into the subject a therapeutically effective amount of genetically engineered host cells such as hematopoietic cells, for example, immune cells (e.g., T lymphocytes or NK cells), which co-express a Krebs cycle modulating polypeptide and a CAR polypeptide of the disclosure. The subject (e.g., a human patient such as a human cancer patient) may additionally have been treated or is being treated with an anti-cancer or anti-infection therapy including, but not limited to, an anti-cancer therapeutic agent or anti-infection agent. The CAR has an antigen-binding domain that may bind any target antigen. Such a target antigen may be any molecule that is associated with a disease or condition, including, but are not limited to, tumor antigens, pathogenic antigens (e.g., bacterial, fungal, or viral), or antigens present on diseased cells, such as those described herein.

Host cells (e.g., hematopoietic cells, for example, immune cells such as T cells and NK cells) expressing Krebs cycle modulating polypeptides and CAR polypeptides described herein are useful for inhibiting cells expressing a target antigen and/or for enhancing growth and/or proliferation of immune cells in a low-glucose environment, a low amino acid environment, a low pH environment, and/or a hypoxic environment, for example, in a tumor microenvironment. In some embodiments, the subject is a mammal, such as a human, monkey, mouse, rabbit, or domestic mammal. In some embodiments, the subject is a human. In some embodiments, the subject is a human cancer patient. In some embodiments, the subject has additionally been treated or is being treated with any of the therapeutic antibodies described herein.

To practice the method described herein, an effective amount of the hematopoietic cells, for example, immune cells (NK cells and/or T lymphocytes) expressing any of the Krebs cycle modulating polypeptides and the CAR polypeptides described herein, or compositions thereof may be administered to a subject in need of the treatment via a suitable route, such as intravenous administration. As used herein, an effective amount refers to the amount of the respective agent (e.g., the NK cells and/or T lymphocytes expressing Krebs cycle modulating polypeptides, CAR polypeptides, or compositions thereof) that upon administration confers a therapeutic effect on the subject. Determination of whether an amount of the cells or compositions described herein achieved the therapeutic effect would be evident to one of skill in the art. Effective amounts vary, as recognized by those skilled in the art, depending on the particular condition being treated, the severity of the condition, the individual patient parameters including age, physical condition, size, gender, sex, and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human. In some embodiments, the subject in need of treatment is a human cancer patient. In some embodiments, the subject in need of treatment suffers from one or more pathogenic infections (e.g., viral, bacterial, and/or fungal infections).

The methods of the disclosure may be used for treatment of any cancer or any pathogen. Specific non-limiting examples of cancers which can be treated by the methods of the disclosure include, for example, lymphoma, breast cancer, gastric cancer, neuroblastoma, osteosarcoma, lung cancer, skin cancer, prostate cancer, colorectal cancer, renal cell carcinoma, ovarian cancer, rhabdomyosarcoma, leukemia, mesothelioma, pancreatic cancer, head and neck cancer, retinoblastoma, glioma, glioblastoma, thyroid cancer, hepatocellular cancer, esophageal cancer, and cervical cancer. In certain embodiments, the cancer may be a solid tumor.

The methods of this disclosure may also be used for treating infectious diseases, which may be caused by bacterial infection, viral infection, or fungus infection. In such instances, genetically engineered immune cells expressing a CAR polypeptide specific to a pathogenic antigen, (e.g., an antigen associated with the bacterium, virus, or fungus that causes the infection) can be used to eliminate infected cells. Specific non-limiting examples of pathogenic antigens include, but are not limited to, bacterial, viral, and/or fungal antigens.

In some embodiments, the immune cells are administered to a subject in an amount effective in inhibiting cells expressing the target antigen by least 20% and/or by at least 2-fold, e.g., inhibiting cells expressing the target antigen by 50%, 80%, 100%, 2-fold, 5-fold, 10-fold, 20-fold, 50-fold, 100-fold, or more.

Additional therapeutic agents (e.g., antibody-based immunotherapeutic agents) may be used to treat, alleviate, or reduce the symptoms of any disease or disorder for which the therapeutic agent is considered useful in a subject.

The efficacy of the cell-based immunotherapy as described herein may be assessed by any method known in the art and would be evident to a skilled medical professional. For example, the efficacy of the cell-based immunotherapy may be assessed by survival of the subject or tumor or cancer burden in the subject or tissue or sample thereof. In some embodiments, the immune cells are administered to a subject in need of the treatment in an amount effective in enhancing the efficacy of an cell-based immunotherapy by at least 20% and/or by at least 2-fold, e.g., enhancing the efficacy of an antibody-based immunotherapy by 50%, 80%, 100%, 2-fold, 5-fold, 10-fold, 20-fold, 50-fold, 100-fold or more, as compared to the efficacy in the absence of the immune cells expressing the Krebs cycle modulating polypeptide and/or the CAR polypeptide.

In any of the compositions or methods described herein, the immune cells (e.g., NK and/or T cells) may be autologous to the subject, i.e., the immune cells may be obtained from the subject in need of the treatment, genetically engineered for expression of the Krebs cycle modulating polypeptides and/or the CAR polypeptides, and then administered to the same subject. In one specific embodiment, prior to re-introduction into the subject, the autologous immune cells (e.g., T lymphocytes or NK cells) are activated and/or expanded ex vivo. Administration of autologous cells to a subject may result in reduced rejection of the host cells as compared to administration of non-autologous cells.

Alternatively, the host cells are allogeneic cells, i.e., the cells are obtained from a first subject, genetically engineered for expression of the Krebs cycle modulating polypeptide and/or the chimeric receptor polypeptide (e.g., ACTR polypeptide or CAR polypeptide), and administered to a second subject that is different from the first subject but of the same species. For example, allogeneic immune cells may be derived from a human donor and administered to a human recipient who is different from the donor. In a specific embodiment, the T lymphocytes are allogeneic T lymphocytes in which the expression of the endogenous T cell receptor has been inhibited or eliminated. In one specific embodiment, prior to introduction into the subject, the allogeneic T lymphocytes are activated and/or expanded ex vivo. T lymphocytes can be activated by any method known in the art, e.g., in the presence of anti-CD3/CD28, IL-2, phytohemoagglutinin, engineered artificial stimulatory cells or particles, or a combination thereof.

NK cells can be activated by any method known in the art, e.g., in the presence of one or more agents selected from the group consisting of CD137 ligand protein, CD137 antibody, IL-15 protein, IL-15 receptor antibody, IL-2 protein, IL-12 protein, IL-21 protein, and K562 cell line, and/or engineered artificial stimulatory cells or particles. Sec, e.g., U.S. Pat. Nos. 7,435,596 and 8,026,097 for the description of useful methods for expanding NK cells. For example, NK cells used in the compositions or methods of the disclosure may be preferentially expanded by exposure to cells that lack or poorly express major histocompatibility complex I and/or II molecules and which have been genetically modified to express membrane bound IL-15 and 4-1BB ligand (CDI37L). Such cell lines include, but are not necessarily limited to, K562 [ATCC, CCL 243; Lozzio et al., Blood 45 (3): 321-334 (1975); Klein et al., Int. J. Cancer 18:421-431 (1976)], and the Wilms tumor cell line HFWT (Fchniger et al., Int Rev Immunol 20 (3-4): 503-534 (2001); Harada H, et al., Exp Hematol 32 (7): 614-621 (2004)), the uterine endometrium tumor cell line HHUA, the melanoma cell line HMV-II, the hepatoblastoma cell line HuH-6, the lung small cell carcinoma cell lines Lu-130 and Lu-134-A, the neuroblastoma cell lines NB 19 and N1369, the embryonal carcinoma cell line from testis NEC 14, the cervix carcinoma cell line TCO-2, and the bone marrow-metastasized neuroblastoma cell line TNB 1 [Harada, et al., Jpn. J. Cancer Res 93:313-319 (2002)]. Preferably the cell line used lacks or poorly expresses both MHC I and II molecules, such as the K562 and HFWT cell lines. A solid support may be used instead of a cell line. Such support should preferably have attached on its surface at least one molecule capable of binding to NK cells and inducing a primary activation event and/or a proliferative response or capable of binding a molecule having such an affect thereby acting as a scaffold. The support may have attached to its surface the CD137 ligand protein, a CD137 antibody, the IL-15 protein or an IL-15 receptor antibody. Preferably, the support will have IL-15 receptor antibody and CD137 antibody bound on its surface.

In one embodiment of the described compositions or methods, introduction (or re-introduction) of T lymphocytes, NK cells, or T lymphocytes and NK cells to the subject is followed by administering to the subject a therapeutically effective amount of IL-2.

In accordance with the present disclosure, patients can be treated by infusing therapeutically effective doses of immune cells such as T lymphocytes or NK cells comprising a Krebs cycle modulating polypeptide and/or a CAR polypeptide of the disclosure in the range of about 10⁵ to 10¹⁰ or more cells per kilogram of body weight (cells/Kg). The infusion can be repeated as often and as many times as the patient can tolerate until the desired response is achieved. The appropriate infusion dose and schedule will vary from patient to patient, but can be determined by the treating physician for a particular patient. Typically, initial doses of approximately 10⁶ cells/Kg will be infused, escalating to 10⁸ or more cells/Kg. IL-2 can be co-administered to expand infused cells. The amount of IL-2 can about 1-5×10⁶ international units per square meter of body surface.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The efficacy of the compositions or methods described herein may be assessed by any method known in the art and would be evident to a skilled medical professional. For example, the efficacy of the compositions or methods described herein may be assessed by survival of the subject or cancer or pathogen burden in the subject or tissue or sample thereof. In some embodiments, the compositions and methods described herein may be assessed based on the safety or toxicity of the therapy (e.g., administration of the immune cells expressing the Krebs cycle modulating polypeptides and the CAR polypeptides) in the subject, for example, by the overall health of the subject and/or the presence of adverse events or severe adverse events.

### (c) Other Immunotherapies

In some embodiments, the genetically-engineered immune cells, expressing one or more of the Krebs cycle modulating polypeptides (e.g., GOT such as GOT1 or GOT2), may be derived from natural immune cells specific to diseased cells (e.g., cancer cells or pathogen infected cells). Such genetically-engineered immune cells (e.g., tumor-infiltrating lymphocytes or TILs) may not co-express any chimeric receptor polypeptide and can be used to destroy the target disease cells, e.g., cancer cells. The genetically-engineered TILs, expressing one or more Krebs cycle modulating polypeptides but not chimeric receptors, may be co-used with a bispecific antibody capable of binding to the target tumor cells and the TILs (BiTE).

In some embodiments, the genetically-engineered immune cells, expressing one or more of the Krebs cycle modulating polypeptides (e.g., GOT such as GOT1 or GOT2), may be Treg cells. Such Treg cells may co-express an chimeric receptor polypeptide as disclosed herein. Alternatively, the Treg cells may not co-express any chimeric receptor polypeptide and can be used for the intended therapy.

### V. Combination Treatments

The compositions and methods described in the present disclosure may be utilized in conjunction with other types of therapy for cancer, such as chemotherapy, surgery, radiation, gene therapy, and so forth, or anti-infection therapy. Such therapies can be administered simultaneously or sequentially (in any order) with the immunotherapy according to the present disclosure. When co-administered with an additional therapeutic agent, suitable therapeutically effective dosages for each agent may be lowered due to the additive action or synergy.

In some instances, the immune cells (e.g., T lymphocytes and/or NK cells) expressing any of the Krebs cycle modulating polypeptides and/or the chimeric receptor polypeptides disclosed herein may be administered to a subject who has been treated or is being treated with an additional therapeutic agent (e.g., an additional anti-cancer therapeutic agent). For example, the immune cells may be administered to a human subject simultaneously with the additional therapeutic agent. Alternatively, the immune cells may be administered to a human subject before the additional therapeutic agent. Alternatively, the immune cells may be administered to a human subject after the additional therapeutic agent.

Genetically engineered immune cells (e.g., T cells or NK cells) that co-express a Krebs cycle modulating polypeptide and a CAR polypeptide specific to a tag can be co-used with a therapeutic agent conjugated to the tag. Via the therapeutic agent, which is capable of binding to an antigen associated with diseased cells such as tumor cells, such genetically engineered immune cells can be engaged with the diseased cells and inhibit their growth. Any of the antibodies listed in Table 1 above, or others specific to the same target antigen also listed in Table I can be conjugated to a suitable tag (e.g., those described herein) and be co-used with immune cells co-expressing the Krebs cycle modulating polypeptide and a CAR polypeptide specific to the tag.

The treatments of the disclosure can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.).

Non-limiting examples of other therapeutic agents useful for combination with the immunotherapy of the disclosure include: (i) anti-angiogenic agents (e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000)); (ii) a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof; and (iii) chemotherapeutic compounds such as, e.g., pyrimidine analogs (5-fluorouracil, floxuridine, capecitabine, gemcitabine and cytarabine), purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones, and navelbine, epidipodophyllotoxins (etoposide and teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamine oxaliplatin, iphosphamide, melphalan, merchlorchtamine, mitomycin, mitoxantrone, nitrosourca, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycin, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (brefeldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); AKT inhibitors (such as MK-2206 2HCl, Perifosinc (KRX-0401), GSK690693, Ipatasertib (GDC-0068), AZD5363, uprosertib, afuresertib, or triciribine); mTOR inhibitors, topoisomerase inhibitors (doxorubicin (adriamycin), amsacrine, camptothecin, daunorubicin, dactinomycin, eniposide, epirubicin, etoposide, idarubicin, mitoxantrone, topotecan, and irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; and chromatin disruptors.

For examples of additional useful agents see also Physician's Desk Reference, 59.sup.th edition, (2005), Thomson P D R, Montvale N.J.; Gennaro et al., Eds. Remington's The Science and Practice of Pharmacy 20th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

The administration of an additional therapeutic agent can be performed by any suitable route, including systemic administration as well as administration directly to the site of the disease (e.g., to a tumor).

In some embodiments, the method involves administering the additional therapeutic agent (e.g., an antibody) to the subject in one dose. In some embodiments, the method involves administering the additional therapeutic agent (e.g., an antibody) to the subject in multiple doses (e.g., at least 2, 3, 4, 5, 6, 7, or 8 doses). In some embodiments, the additional therapeutic agent (e.g., an antibody) is administered to the subject in multiple doses, with the first dose of the additional therapeutic agent (e.g., an antibody) administered to the subject about 1, 2, 3, 4, 5, 6, or 7 days prior to administration of the immune cells expressing the Krebs cycle modulating polypeptide and/or the CAR polypeptide. In some embodiments, the first dose of the additional therapeutic agent (e.g., an antibody) is administered to the subject between about 24-48 hours prior to the administration of the immune cells expressing the Krebs cycle modulating polypeptide and/or the CAR polypeptide. In some instances, the additional therapeutic agent can be an antibody specific to a target antigen of interest, for example, those listed in Table 1 and others that are specific to the same target.

In some embodiments, the additional therapeutic agent (e.g., an antibody) is administered to the subject prior to administration of the immune cells expressing the Krebs cycle modulating polypeptide and/or the CAR polypeptide and then subsequently about every two weeks. In some embodiments, the first two doses of the additional therapeutic agent (e.g., an antibody) are administered about one week (e.g., about 6, 7, 8, or 9 days) apart. In certain embodiments, the third and following doses are administered about every two weeks.

In any of the embodiments described herein, the timing of the administration of the additional therapeutic agent (e.g., an antibody) is approximate and includes three days prior to and three days following the indicated day (e.g., administration every three weeks encompasses administration on day 18, day 19, day 20, day 21, day 22, day 23, or day 24).

The efficacy of the methods described herein may be assessed by any method known in the art and would be evident to a skilled medical professional and/or those described herein. For example, the efficacy of the antibody-based immunotherapy may be assessed by survival of the subject or cancer burden in the subject or tissue or sample thereof. In some embodiments, the antibody-based immunotherapy is assessed based on the safety or toxicity of the therapy in the subject, for example by the overall health of the subject and/or the presence of adverse events or severe adverse events.

### VI. Kits for Therapeutic Use

The present disclosure also provides kits for use of the compositions described herein. For example, the present disclosure also provides kits comprising a population of immune cells (e.g., T lymphocytes or NK cells, constructed in vitro or in vivo) that express a Krebs cycle modulating polypeptide and optionally a chimeric receptor polypeptide for use in inhibiting the growth of diseased cells, e.g., tumor cells and/or enhancing immune cell growth and/or proliferation in a low glucose environment, a low amino acid environment, a low-pH environment, and/or hypoxic environment, for example, in a tumor microenvironment. The kit may further comprise a therapeutic agent or a therapeutic agent conjugated to a tag (e.g., those described herein), to which the chimeric receptor polypeptide expressed on the immune cells bind. Such kits may include one or more containers comprising the population of the genetically engineered immune cells as described herein (e.g., T lymphocytes and/or NK cells), which co-express a Krebs cycle modulating polypeptide and a chimeric receptor polypeptide such as those described herein, and optionally a therapeutic agent or a therapeutic agent conjugated to a tag.

In some embodiments, the kit described herein comprises Krebs cycle modulating polypeptide-expressing and chimeric receptor polypeptide-expressing immune cells, which are expanded in vitro, and an antibody specific to a cell surface antibody that is present on activated T cells, for example, an anti-CD5 antibody, an anti-CD38 antibody or an anti-CD7 antibody. The Krebs cycle modulating polypeptide-expressing and chimeric receptor polypeptide-expressing immune cells may express any of the chimeric receptor polypeptide constructs known in the art or disclosed herein.

Alternatively, the kit disclosed herein may comprise a nucleic acid or a nucleic acid set as described herein, which collectively encodes any of the chimeric receptor polypeptides and any of the Krebs cycle modulating polypeptides as also described herein.

In some embodiments, the kit can additionally comprise instructions for use in any of the methods described herein. The included instructions may comprise a description of administration of the first and second pharmaceutical compositions to a subject to achieve the intended activity, e.g., inhibiting target cell growth in a subject, and/or enhancing the growth and/or proliferation of immune cells in a low-glucose environment, a low amino acid (e.g., a low glutamine environment) environment, a low pH environment, and/or a hypoxic environment (e.g., a low glucose, low amino acid, low pH or hyposic tumor microenvironment). The kit may further comprise a description of selecting a subject suitable for treatment based on identifying whether the subject is in need of the treatment. In some embodiments, the instructions comprise a description of administering the population of genetically engineered immune cells and optionally a description of administering the tag-conjugated therapeutic agent.

The instructions relating to the use of the immune cells and optionally the tag-conjugated therapeutic agent as described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated arc packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the second pharmaceutical composition is an antibody as described herein. At least one active agent in the first pharmaceutical composition is a population of immune cells (e.g., T lymphocytes or NK cells) that express a chimeric receptor polypeptide and a Krebs cycle modulating polypeptide as described herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, cd. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Impact of Expressing a Krebs Cycle Modulating Polypeptide on T Cell Function in Lower Glucose Environments

A Krebs cycle modulating polypeptide transgene is co-expressed in the same T cell with an ACTR polypeptide. The transgene is, for example, PHGDG, PCK1, IDH1, IDH1, MDH1, MDH2, GOT1, GOT2, PSAT1, GDH1, GPT1, or GLS (e.g., SEQ ID NOs: 81-92). The T cells are transduced with a virus encoding the ACTR polypeptide and the glucose importation polypeptide separated, for example, by a P2A ribosomal skip sequence. The T cells are mixed at a given effector-to-target (E:T) ratio with tumor target cells, such as IGROV-1 cells, and a tumor-targeting antibody such as an anti-FOLR1 antibody. Reactions are then incubated at 37° C. in a 5% CO₂ incubator for a period of time (e.g., 6-8 days) at different starting concentrations of glucose (e.g., 0-20 mM). T cell function is then evaluated, for example, using cytokine production or T cell proliferation assays or for resistance to chronic stimulation. Cytokine production (e.g., IL-2 and/or IFN-gamma) is measured from the reaction supernatant. For proliferation experiments, co-cultures are harvested and stained with an anti-CD3 antibody and a live-dead cell stain. The number of live, CD3-positive cells is evaluated by flow cytometry as a measure of T cell proliferation. T cells expressing a Krebs cycle modulating polypeptide in addition to the ACTR polypeptide show enhanced T cell function relative to T cells expressing ACTR alone including, for example, enhanced cytokine production or enhanced proliferation. This enhanced function may be more pronounced at lower glucose concentrations. These experiments demonstrate that expressing a Krebs cycle modulating polypeptide in T cells has a positive impact on T cell activity.

### Example 2: Impact of Expressing a Krebs Cycle Modulating Polypeptide Gene on T Cell Function in Environments with Higher Soluble Inhibitor Concentrations

A Krebs cycle modulating polypeptide transgene is co-expressed in the same T cell with an ACTR polypeptide. The transgene is, for example, PHGDG, PCK1, IDH1, IDH1, MDH1, MDH2, GOT1, GOT2, PSAT1, GDH1, GPT1, or GLS (e.g., SEQ ID NOs: 81-92). The T cells are transduced with virus encoding the ACTR polypeptide and the Krebs cycle modulating polypeptide separated, for example, by a P2A ribosomal skip sequence. Transduced T cells are mixed at a given effector-to-target (E:T) ratio with tumor target cells, such as IGROV-1 cells, and a tumor-targeting antibody such as an anti-FOLR1 antibody, in media containing different concentrations of soluble inhibitors that are present in the tumor microenvironment (e.g., TGF-beta, PGE₂, kynurenine, and/or adenosine). Reactions are then incubated at 37° C. in a 5% CO₂ incubator for a period of time (e.g., 6-8 days). T cell function is then evaluated, for example, using cytokine production or T cell proliferation assays or for resistance to chronic stimulation. Cytokine production (e.g., IL-2 and/or IFN-gamma) is measured from the reaction supernatant. For proliferation experiments, co-cultures are harvested and stained with an anti-CD3 antibody and a live-dead cell stain. The number of live, CD3-positive cells is evaluated by flow cytometry as a measure of T cell proliferation. T cells expressing a Krebs cycle modulating polypeptide in addition to the ACTR polypeptide show enhanced T cell function relative to T cells expressing ACTR alone including, for example, enhanced cytokine production or enhanced proliferation. This enhanced function may be achieved at higher soluble inhibitor concentrations. These experiments demonstrate that expressing a Krebs cycle modulating polypeptide in T cells has a positive impact on T cell activity.

### Example 3: Impact of Expressing a Krebs Cycle Modulating Polypeptide on T Cell Function in Environments with Greater Immunosuppressive Cell Presence

A Krebs cycle modulating polypeptide transgene is co-expressed in the same T cell with an ACTR polypeptide. The transgene is, for example, PHGDG, PCK1, IDH1, IDH1, MDH1, MDH2, GOT1, GOT2, PSAT1, GDH1, GPT1, or GLS (e.g., SEQ ID NOs: 81-92). The T cells are transduced with virus encoding the ACTR polypeptide and the Krebs cycle modulating polypeptide separated, for example, by a P2A ribosomal skip sequence. Transduced T cells are mixed at a given effector-to-target (E:T) ratio with tumor target cells, such as IGROV-1 cells, and a tumor-targeting antibody such as an anti-FOLR1 antibody, in the presence of immunosuppressive cells (e.g., myeloid-derived suppressor cells and/or regulatory T cells). Reactions are then incubated at 37° C. in a 5% CO₂ incubator for a period of time (e.g., 3-10 days). T cell function is then evaluated, for example, using cytokine production or T cell proliferation assays or for resistance to chronic stimulation. Cytokine production (e.g., IL-2 and/or IFN-gamma) is measured from the reaction supernatant. For proliferation experiments, co-cultures are harvested and stained with an anti-CD3 antibody and a live-dead cell stain. The number of live, CD3-positive cells is evaluated by flow cytometry as a measure of T cell proliferation. T cells expressing a Krebs cycle modulating polypeptide in addition to the ACTR polypeptide show enhanced T cell function relative to T cells expressing ACTR alone including, for example, enhanced cytokine production or enhanced proliferation. This enhanced function may be achieved in the presence of increased amounts (e.g., greater number or percentage) of immunosuppressive cells. These experiments demonstrate that expressing a Krebs cycle modulating polypeptide in T cells has a positive impact on T cell activity.

### Example 4: Impact of Expressing a Krebs Cycle Modulating Polypeptide on T Cell Function on Tumor Models

A Krebs cycle modulating polypeptide transgene is co-expressed in the same T cell with an ACTR polypeptide. The transgene is, for example, PHGDG, PCK1, IDH1, IDH1, MDH1, MDH2, GOT1, GOT2, PSAT1, GDH1, GPT1, or GLS (e.g., SEQ ID NOs: 81-92). The T cells are transduced with virus encoding the ACTR polypeptide and the Krebs cycle modulating polypeptide separated, for example, by a P2A ribosomal skip sequence. Transduced T cells are evaluated for anti-tumor activity in mouse tumor models. For these experiments, a tumor cell line, for example IGROV-1, is inoculated into NSG™ (NOD scid gamma, NOD.Cg-Prkdc^{scid} IL2rg^{tm1Wjl}/SzJ, Strain 005557) mice. Tumor-bearing mice are subsequently dosed with a tumor-targeting antibody and T cells expressing ACTR alone or ACTR and a Krebs cycle modulating polypeptide. Tumor growth is monitored throughout the course of the experiment. In combination with a tumor-targeting antibody, T cells expressing a Krebs cycle modulating polypeptide in addition to an ACTR polypeptide show enhanced anti-tumor activity relative to T cells expressing an ACTR polypeptide alone. Additionally, in combination with a tumor-targeting antibody, T cells expressing a Krebs cycle modulating polypeptide in addition to an ACTR polypeptide may show enhanced T cell activity including, for example, enhanced proliferation, enhanced T cell persistence, and/or enhanced cytokine production relative to T cells expressing the ACTR polypeptide alone. These experiments demonstrate that expressing a Krebs cycle modulating polypeptide in ACTR-expressing T cells has a positive impact on T cell function in vivo.

### Example 5: Impact of Reduced Glucose Concentrations on T Cell Function

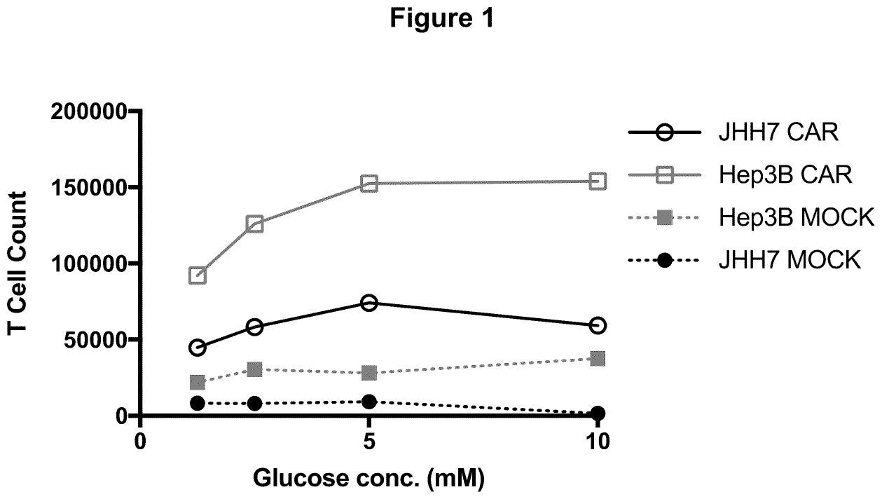

Gamma-retrovirus encoding an exemplary GPC3-targeting CAR expression construct of SEQ ID NO: 104 was generated via recombinant technology and used to infect primary human T-cells for generating cells that express a GPC3-targeting CAR polypeptide on their cell surface. A six-day flow-based proliferation assay was then used to test the functionality of the GPC3-targeting CAR expressing cells. Specifically, 200,000 untransduced mock T-cells or T-cells expressing the GPC3-targeting CAR construct were incubated together at a ratio of 4:1 (effector cells/CAR-expressing T cells to target cells) with either 50,000 GPC3+ hepatocellular carcinoma JHH7 or Hep3B tumor cells. The co-culture was incubated at 37° C. in a 5% CO₂ incubator for six days in the presence of different concentrations of glucose. At the end of six days, co-cultures were harvested and stained with an anti-CD3 antibody. The number of CD3-positive cells was evaluated by flow cytometry as a measure of T cell proliferation. At lower glucose concentrations, less CAR-T proliferation was observed (FIG. 1). These experiments demonstrate that low glucose environments may have a negative impact on CAR-T cell proliferation activity.

### Example 6: Impact of Expressing a Krebs Cycle Modulating Gene on T Cell Function Using a GPC3-Targeting CAR-T Expression Construct

Gamma-retrovirus encoding an exemplary GPC3-targeting CAR polypeptide expression construct (SEQ ID NO: 104) was generated via recombinant technology and used to infect primary human T-cells to generate cells expressing a GPC3-targeting CAR polypeptide on their cell surface. Additionally, gamma-retroviruses encoding an exemplary GPC3-targeting CAR polypeptide and a Krebs cycle modulating polypeptide (GOT1 or GOT2) (SEQ ID NOs: 87 and 88, respectively) were generated via recombinant technology and used to infect primary human T-cells to generate cells that expressed a GPC3-targeting polypeptide and a Krebs cycle modulating polypeptide. In the constructs encoding both the CAR polypeptide and the Krebs cycle modulating polypeptide, the two polypeptides were separated by a P2A ribosomal skip sequence. The variants expressed were anti-GPC3 CAR+GOT1 and anti-GPC3 CAR+GOT2. A six-day flow-based proliferation assay was then used to test the functionality of the GPC3-targeting CAR expressing cells. Specifically, 200,000 untransduced mock T-cells, T-cells expressing a GPC3-targeting CAR polypeptide, or T-cells expressing a GPC3-targeting CAR polypeptide and a Krebs cycle modulating polypeptide were incubated together at a ratio of 4:1 (effector CAR-expressing T cells to target cells) with 50,000 GPC3⁺ hepatocellular carcinoma JHH7 tumor cells. The co-culture was incubated at 37° C. in a 5% CO₂ for six days in the presence of 1.25 mM glucose (tumor-relevant) and 10 mM glucose (approximate peripheral blood levels). At the end of six days, co-cultures were harvested and stained with anti-CD3 antibody.

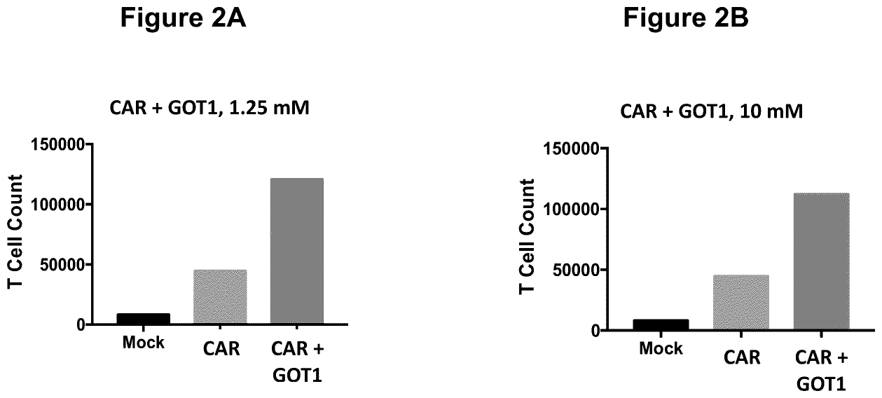

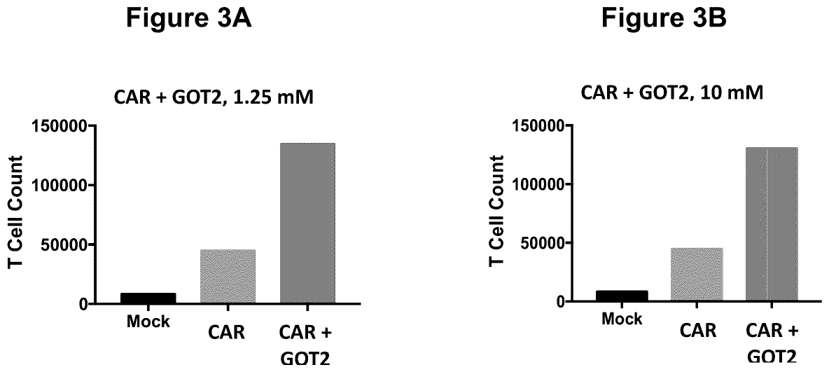

The number of CD3-positive cells was evaluated by flow cytometry as a measure of T cell proliferation. T cells expressing the Krebs cycle modulating polypeptides in addition to the CAR polypeptide demonstrated enhanced T cell proliferation relative to T cells expressing the CAR construct alone (FIGS. 2A, 2B, 3A, and 3B). This enhanced proliferation also occurred at tumor-relevant low glucose concentrations. These experiments demonstrated that expressing Krebs cycle modulating polypeptides in T cells has a positive impact on CAR-T cell proliferation activity.

### Example 7: Impact of Expressing a Krebs Cycle Modulating Gene on T Cell Function in Lower Glucose Environments

A Krebs cycle modulating transgene is co-expressed in the same T cell with a chimeric antigen receptor (CAR) polypeptide. The transgene is, for example, GPT1, GLS, PCK1, GOT1, GOT2, IDH1, IDH2, MDH1, MDH2, PHGDH, PSAT1, or GDH1 (e.g. SEQ ID NOs: 81-92). The T cells are transduced with a virus encoding the CAR polypeptide and the Krebs cycle metabolite polypeptide separated, for example, by a P2A ribosomal skip sequence. The T cells are mixed at a given effector-to-target (E:T) ratio with tumor target cells, such as HepG2 cells. Reactions are then incubated at 37° C. in a 5% CO₂ incubator for a period of time (e.g., 6-8 days) at different starting concentrations of glucose (e.g., 0-20 mM). T cell function is then evaluated, for example, using cytokine production or T cell proliferation assays. Cytokine production (e.g., IL-2 and/or IFN-gamma) is measured from the reaction supernatant. For proliferation experiments, co-cultures are harvested and stained with an anti-CD3 antibody and a live-dead cell stain. The number of live, CD-3 positive cells is evaluated by flow cytometry as a measure of T cell proliferation.

T cells expressing a Krebs cycle modulating polypeptide in addition to the CAR polypeptide are expected to show enhanced T cell function relative to T cells expressing CAR alone including, for example, enhanced cytokine production or enhanced proliferation. This enhanced function may be more pronounced at lower glucose concentrations.

### Example 8: Impact of Expressing a Krebs Cycle Modulating Gene on T Cell Function in Environments with Higher Soluble Inhibitor Concentrations

A Krebs cycle modulating transgene is co-expressed in the same T cell with a chimeric antigen receptor (CAR) polypeptide. The transgene is, for example, GPT1, GLS, PCK1, GOT1, GOT2, IDH1, IDH2, MDH1, MDH2, PHGDH, PSAT1, or GDH1 (e.g., SEQ ID NOs: 81-92). The T cells are transduced with virus encoding the CAR polypeptide and the Krebs cycle modulating polypeptide separated, for example, by a P2A ribosomal skip sequence. Transduced T cells are mixed at a given effector-to-target (E:T) ratio with tumor target cells, such as HepG2 cells, in media containing different concentrations of soluble inhibitors that are present in the tumor microenvironment (e.g., TGF-beta, PGE₂, and/or adenosine). Reactions are then incubated at 37° C. in a 5% CO₂ incubator for a period of time (e.g., 6-8 days). T cell function is then evaluated, for example, using cytokine production or T cell proliferation assays. Cytokine production (e.g., IL-2 and/or IFN-gamma) is measured from the reaction supernatant. For proliferation experiments, co-cultures are harvested and stained with an anti-CD3 antibody and a live-dead cell stain. The number of live, CD3-positive cell is evaluated by flow cytometry as a measure of T cell proliferation.

T cells expressing a Krebs cycle modulating polypeptide in addition to the CAR polypeptide are expected to show enhanced T cell function relative to T cells expressing CAR alone including, for example, enhanced cytokine production or enhanced proliferation. This enhanced function may be achieved at higher soluble inhibitor concentrations.

### Example 9: Impact of Expressing a Krebs Cycle Modulating Gene on T Cell Function in Environments with Greater Immunosuppressive Cell Presence

A Krebs cycle metabolite shunting transgene is co-expressed in the same T cell with a chimeric antigen receptor (CAR) polypeptide. The transgene is, for example, GPT1, GLS, PCK1, GOT1, GOT2, IDH1, IDH2, MDH1, MDH2, PHGDH, PSAT1, or GDH1 (e.g., SEQ ID NOs: 81-92). The T cells are transduced with virus encoding the CAR polypeptide and the Krebs cycle metabolite shunting polypeptide separated, for example, by a P2A ribosomal skip sequence. Transduced T cells are mixed at a given effector-to-target (E:T) ratio with tumor target cells, such as HepG2 cells, in the presence of immunosuppressive cells (e.g, myeloid-derived suppressor cells and/or regulatory T cells) marked with a fluorescent label. Reactions are then incubated at 37° C. in a 5% CO₂ incubator for a period of time (e.g., 3-10 days). T cell function is then evaluated, for example, using cytokine production or T cell proliferation assays. Cytokine production (e.g., IL-2 and/or IFN-gamma) is measured from the reaction supernatant. For proliferation experiments, co-cultures are harvested and stained with an anti-CD3 antibody and a live-dead cell stain. The number of live, CD3-positive, unlabeled cells is evaluated by flow cytometry as a measure of T cell proliferation.

T cells expressing a Krebs cycle modulating polypeptide in addition to the CAR polypeptide are expected to show enhanced T cell function relative to T cells expressing CAR alone including, for example, enhanced cytokine production or enhanced proliferation. This enhanced function may be achieved in the presence of increased amounts (e.g., greater number or percentage) of immunosuppressive cells.

### Example 10: Impact of Expressing a Krebs Cycle Modulating Polypeptide on T Cell Function in a Mouse Tumor Model Using a GPC3-Targeting CAR-T Expression Construct

A Krebs cycle modulating polypeptide (GOT2) (SEQ ID NO: 88) was co-expressed in the same T cells with a GPC3-targeting CAR-T polypeptide (SEQ ID NO:104). Gamma-retrovirus encoding the GPC3-targeting CAR-T expression construct was generated and used to infect primary human T-cells to generate cells that expressed a GPC3-targeting CAR-T on their cell surface. The T cells were also transduced with virus encoding the anti-GPC3 CAR polypeptide and GOT2 separated by a P2A ribosomal skip sequence.

The CAR transduced T cells were evaluated for anti-tumor activity in a mouse tumor model. For these experiments, the hepatocellular carcinoma tumor cell line, JHH7, was inoculated into NSG™ (NOD scid gamma, NOD.Cg-Prkdc^{scid} IL2rg^{tmWj1}/SzJ, Strain 005557) mice. Treatment with anti-GPC3 CAR-expressing T cells was initiated when tumor volumes reached approximately 50 mm³ (day 8 post inoculation). Mice were randomized into treatment groups of 5 mice each based on tumor volume, and treated with T cells expressing the GPC3-targeted CAR at a dose of 5×10⁶ CAR+ T cells on days 8 and 15 post inoculation. The total T cell dose varied based on the CAR transduction efficiency of each construct.

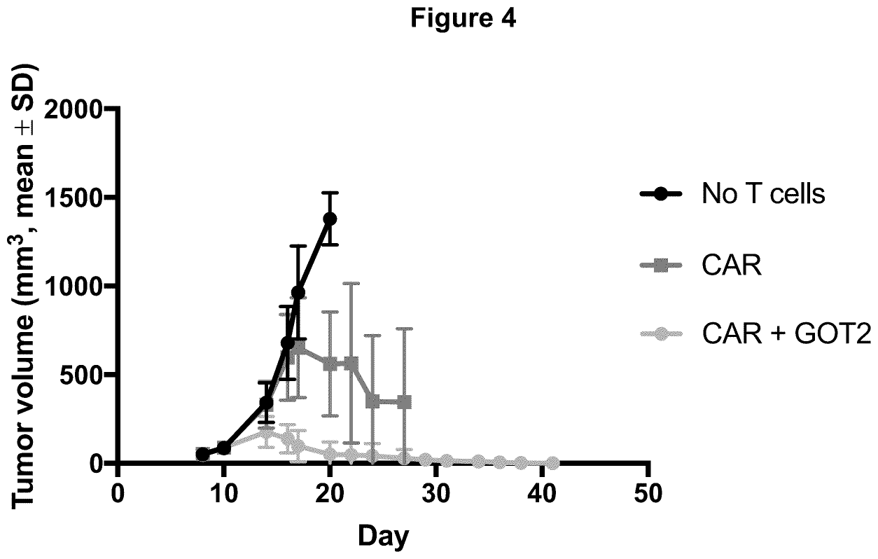

Tumor volume and body weights were measured two-to-three times weekly for the duration of the experiment. CAR-T cells co-expressing GOT2 demonstrated enhanced anti-tumor efficacy relative to the T cells only expressing the GPC3 CAR construct (FIG. 4).

These experiments demonstrated that expressing the Krebs cycle modulating polypeptide in CAR-T cells had a positive impact on CAR-T cell anti-tumor efficacy in a mouse xenograft model of hepatocellular carcinoma.

The CAR transduced T cells were evaluated for anti-tumor activity in a mouse tumor model. For these experiments, the hepatocellular carcinoma tumor cell line, Hep3B, was inoculated into NSG™ (NOD scid gamma, NOD.Cg-Prkdc^{scid} IL2rg^{tmWj1}/SzJ, Strain 005557) mice. Treatment with anti-GPC3 CAR-expressing T cells was initiated when tumor volumes reached approximately 100 mm³ (day 20 post inoculation). Mice were randomized into treatment groups of 5 mice each based on tumor volume, and treated with T cells expressing the GPC3-targeted CAR at a dose of 1×10⁶ CAR+ T cells on days 20 and 27 post inoculation. The total T cell dose varied based on the CAR transduction efficiency of each construct.

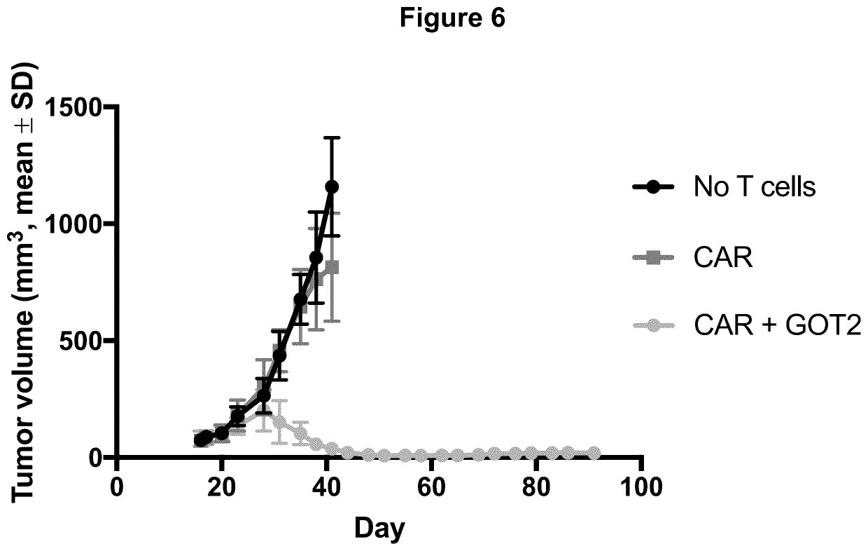

Tumor volume and body weights were measured two-to-three times weekly for the duration of the experiment. CAR-T cells co-expressing GOT2 demonstrated enhanced anti-tumor efficacy relative to the T cells only expressing the GPC3 CAR construct (FIG. 6). These experiments demonstrated that expressing the Krebs cycle modulating polypeptide in CAR-T cells had a positive impact on CAR-T cell anti-tumor efficacy in a mouse xenograft model of hepatocellular carcinoma.

The CAR transduced T cells were evaluated for anti-tumor activity in a mouse tumor model. For these experiments, the small cell lung cancer tumor cell line, NCI-H446, was inoculated into NSG™ (NOD scid gamma, NOD.Cg-Prkdc^{scid} IL2rg^{tmWj1}/SzJ, Strain 005557) mice. Treatment with anti-GPC3 CAR-expressing T cells was initiated when tumor volumes reached approximately 100 mm³ (day 18 post inoculation). Mice were randomized into treatment groups of 5 mice each based on tumor volume, and treated with T cells expressing the GPC3-targeted CAR at a dose of 5×10⁶ CAR+ T cells on days 18 and 25 post inoculation. The total T cell dose varied based on the CAR transduction efficiency of each construct.

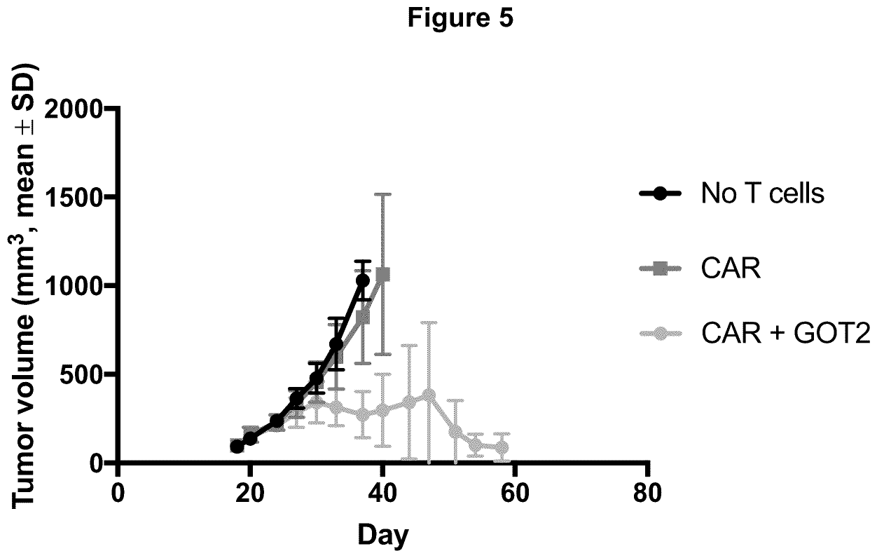

Tumor volume and body weights were measured two-to-three times weekly for the duration of the experiment. CAR-T cells co-expressing GOT2 demonstrated enhanced anti-tumor efficacy relative to the T cells only expressing the GPC3 CAR construct (FIG. 5). These experiments demonstrated that expressing the Krebs cycle modulating polypeptide in CAR-T cells had a positive impact on CAR-T cell anti-tumor efficacy in a mouse xenograft model of small cell lung cancer.

### Example 11: Impact of Expressing a Krebs Cycle Modulating Polypeptide on T Cell Expansion in a Mouse Tumor Model Using a GPC3-Targeting CAR-T Expression Construct

A Krebs cycle modulating polypeptide (GOT2) (SEQ ID NO: 88) was co-expressed in the same T cells with a GPC3-targeting CAR-T polypeptide (SEQ ID NO: 104). Gamma-retrovirus encoding the GPC3-targeting CAR-T expression construct was generated and used to infect primary human T-cells to generate cells that expressed a GPC3-targeting CAR-T on their cell surface. The T cells were also transduced with virus encoding the anti-GPC3 CAR polypeptide and GOT2 separated by a P2A ribosomal skip sequence.

The CAR-transduced T cells were evaluated for expansion and in a mouse tumor model. For these experiments, the hepatocellular carcinoma tumor cell line, Hep3B, was inoculated into NSG™ (NOD scid gamma, NOD.Cg-Prkdc^{scid} IL2rg^{tmWj1}/SzJ, Strain 005557) mice. Treatment with anti-GPC3 CAR-expressing T cells was initiated when tumor volumes reached approximately 100 mm³ (day 20 post inoculation). Mice were randomized into treatment groups of 5 mice each based on tumor volume, and treated with T cells expressing the GPC3-targeted CAR at a dose of 1×10⁶ CAR+ T cells on days 20 and 27 post inoculation. The total T cell dose varied based on the CAR transduction efficiency of each construct.

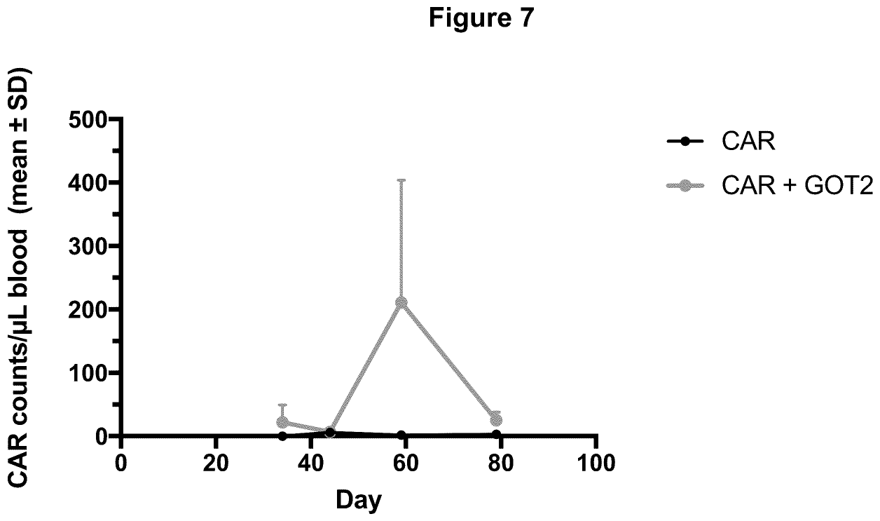

Peripheral blood was collected and stained with an anti-CD3 and an anti-CAR antibody. The number of CD3-positive and CAR-positive cells was evaluated by flow cytometry as a measure of T cell expansion and CAR activity. CAR-T cells co-expressing GOT2 demonstrated enhanced T cell expansion relative to the T cells only expressing the GPC3 CAR construct (FIG. 7). These experiments demonstrated that expressing the Krebs cycle modulating polypeptide in CAR-T cells had a positive impact on CAR-T cell expansion in a mouse xenograft model of hepatocellular carcinoma.

### Example 12: Co-Expression of a Krebs Cycle Modulating Gene (GOT2) and a GPC3-Targeting CAR in T Cells Increases Aspartate Aminotransferase Enzyme Activity

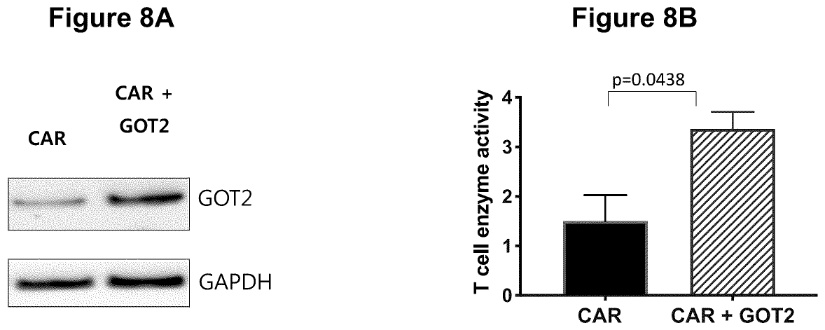

Gamma-retrovirus encoding an exemplary GPC3-targeting CAR polypeptide expression construct (SEQ ID NO: 104) was generated via recombinant technology and used to infect primary human T-cells to generate cells expressing a GPC3-targeting CAR polypeptide on their cell surface. Additionally, gamma-retrovirus encoding an exemplary GPC3-targeting CAR polypeptide and a Krebs cycle modulating polypeptide (GOT2) (SEQ ID NO: 88) was generated via recombinant technology and used to infect primary human T-cells to generate cells that expressed a GPC3-targeting polypeptide and GOT2, with the two polypeptides separated by a P2A ribosomal skip sequence. Expression of GOT2 was confirmed using western blot. CAR transduced T cells were mixed at a 4:1 effector-to-target ratio with GPC3+ hepatocellular carcinoma Hep3B tumor cells and incubated at 37° C., 5% CO₂ for 4 days. Following incubation, cell lysates were prepared from 1×10⁶ cells, cell lysate was combined with Bolt LDS sample buffer (Invitrogen) and 100 nM DTT and run on two separate gels. Proteins from the gels were then transferred onto nitrocellulose membranes (Transblot turbo transfer pack from BioRad). Blots were probed overnight for GOT2 expression using rabbit anti-human GOT2 antibody (Origene) and then probed with HRP-conjugated anti-rabbit antibody or for GAPDH expression using mouse anti-human GAPDH (Biolegend) and then probed with HRP-conjugated anti-mouse IgG secondary antibody. GOT2 expression was greater in T cells transduced with the CAR+GOT2 construct relative to the parental CAR (FIG. 8A)

Transduced T cells were mixed at a 4:1 effector-to-target ratio with GPC3+ hepatocellular carcinoma Hep3B tumor cells and incubated at 37° C., 5% CO₂ for 8 days. Aspartate aminotransferase (AST) enzyme activity was measured using an Aspartate Aminotransferase Activity Assay Kit (Abcam) according to manufacturer's protocol. AST activity is calculated by measuring the amount of glutamate generated per minute at 37° C. In this example, AST activity was calculated at 50 minutes after addition of substrate. T cells expressing the Krebs-cycle-modulating-polypeptide GOT2 in addition to the CAR polypeptide demonstrated increased AST activity following activation with tumor cells. (FIG. 8B). These experiments demonstrate that expressing a Krebs cycle modulating polypeptide in T cells has a positive impact on T cell function.

### Example 13: Co-Expression of a Krebs Cycle Modulating Gene (GOT2) and Either a GPC3-Targeting CAR or ACTR in T Cells Enhances Proliferation

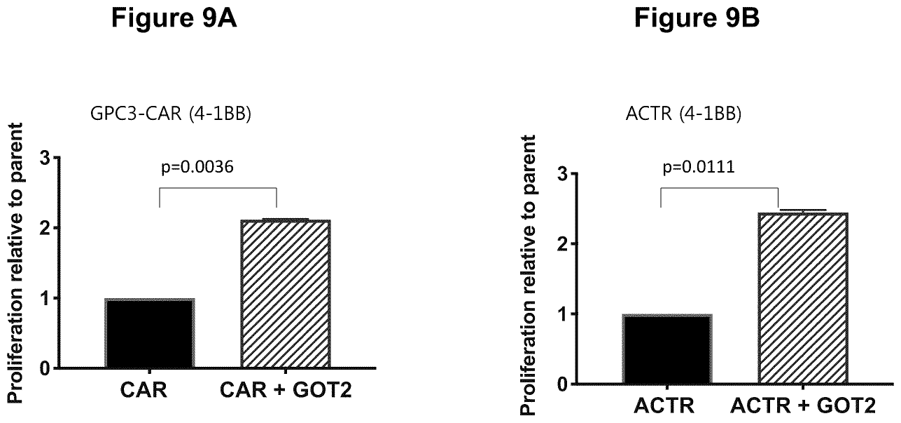

A Krebs cycle modulating polypeptide (GOT2) (SEQ ID NO: 10) was co-expressed in the same T cells with a GPC3-targeting CAR-T polypeptide (SEQ ID NO:1). The T cells were transduced with virus encoding the CAR polypeptide alone or CAR and GOT2 separated by a P2A ribosomal skip sequence. T cells expressing GPC3-targeting CAR and co-expressing GPC3-CAR and GOT2 were labeled with cell trace violet, fluorescently labeling cellular proteins, and then mixed at a 2:1 effector-to-target ratio with GPC3+ hepatocellular carcinoma Hep3B tumor cells. On day 6 after activation, cells were stained with anti-CD3 and cell trace violet dilution was measured by flow cytometry on day 6 after activation to measure proliferation. The inverse of cell trace violet mean fluorescence intensity of CD3+ cells of T cells co-expressing CAR and GOT2 was expressed relative to mean inverse fluorescence intensity of T cells expressing CAR alone. T cells expressing GOT2 in combination with the CAR polypeptide demonstrated increased cell divisions relative to T cells expressing the CAR alone. FIG. 9A

A Krebs cycle modulating polypeptide (GOT2) (SEQ ID NO: 10) was co-expressed in the same T cells with an ACTR polypeptide (SEQ ID NO:1). The T cells were transduced with virus encoding the ACTR polypeptide alone or ACTR and GOT2 separated by a P2A ribosomal skip sequence. T cells expressing ACTR and co-expressing ACTR and GOT2 were mixed at a 2:1 effector-to-target ratio with GPC3+ hepatocellular carcinoma HepG2 tumor cells and 1 μg/mL anti-GPC3 antibody GC33 (see Nakano, K et al. Anticancer Drugs. 2010 November; 21 (10): 907-16). On day 3 after activation, T cell counts were measured by flow cytometry after staining with an anti-CD3 antibody. Total counts of CD3+ cells of T cells co-expressing ACTR and GOT2 was expressed relative to total CD3+ T cell counts of T cells expressing ACTR alone. T cells expressing the GOT2 in addition to the ACTR polypeptide demonstrated increased T cell count. FIG. 9B.

These experiments demonstrate that expressing a Krebs cycle modulating polypeptide in T cells has a positive impact on T cell proliferation.

### Example 14: Co-Expression of a Krebs Cycle Modulating Gene (GOT2) and Either a GPC3-Targeting CAR or ACTR in T Cells Enhances IL-17A Cytokine Production

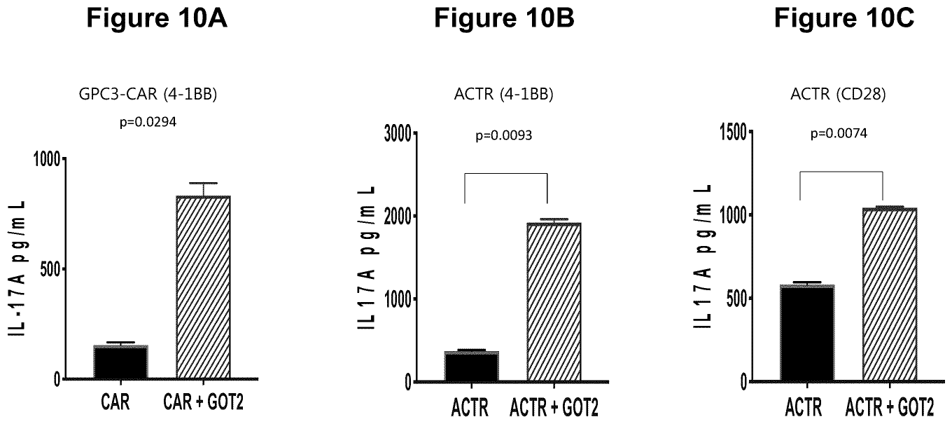

A Krebs cycle modulating polypeptide (GOT2) (SEQ ID NO: 88) was co-expressed in T cells with a GPC3-targeting CAR-T polypeptide (SEQ ID NO: 104) or was co-expressed T cells with an ACTR-4-1BB polypeptide (SEQ ID NO:1) or an ACTR-CD28 polypeptide (SEQ ID NO: 57). The T cells were transduced with virus encoding the CAR or ACTR polypeptide alone or CAR or ACTR and GOT2 separated by a P2A ribosomal skip sequence. Transduced T cells were mixed at a 2:1 effector-to-target ratio with GPC3+ hepatocellular carcinoma HepG2 tumor cells; in experiments with ACTR T cells, the anti-GPC3 antibody GC33 (1 μg/mL) was added. The cells were incubated for 24 hours at 37° C. in a 5% CO₂ incubator. Following the incubation, 100 μL of cell culture supernatant was collected and IL-17A was measured using an MSD ELISA (Meso Scale Discovery, Pacific BioLabs) according to the manufacturer's instructions. IL-17A production is plotted as a function of expression construct (FIGS. 10A-10C). T cells co-expressing GOT2 and CAR (FIG. 10A) or ACTR polypeptides (FIGS. 10B-10C) demonstrated the enhanced ability to produce IL-17A relative to T cells expressing CAR or ACTR alone. These experiments demonstrate that expressing a Krebs cycle modulating polypeptide in T cells has a positive impact on IL-17A cytokine production.

### Example 15: Co-Expression of a Krebs Cycle Modulating Gene (GOT2) and Either a GPC3-Targeting CAR or ACTR Enhances CD4+ T Cell Polyfunctionality

A Krebs cycle modulating polypeptide (GOT2) (SEQ ID NO: 88) was co-expressed in the same T cells with a GPC3-targeting CAR-T polypeptide (SEQ ID NO:104) or was co-expressed in the same T cells with an ACTR polypeptide containing either a 4-1BB (SEQ ID NO: 1) or CD28 (SEQ ID NO:57) primary costimulatory domain. The T cells were transduced with virus encoding the CAR or ACTR polypeptide alone or CAR or ACTR and GOT2 separated by a P2A ribosomal skip sequence. Transduced T cells were mixed at a 1:1 effector-to-target ratio with GPC3+ hepatocellular carcinoma HepG2 tumor cells; in experiments with ACTR T cells, the anti-GPC3 antibody GC33 (1 μg/mL) was added. The cells were incubated in the presence of protein transport inhibitors, Brefeldin A (5 μg/mL) and Monensin (2 pM), for 6 hours at 37° C. in a 5% CO₂ incubator. Following the incubation, co-cultures were stained with anti-CD4 and anti-CD8 antibodies for both CAR and ACTR T cells; ACTR T cells were also stained with an anti-CD16 antibody. The cells were subsequently fixed, permeabilized, and stained with anti-IFNγ, anti-IL-2, anti-TNFα, and anti-IL-17A antibodies to quantify intracellular cytokine production.

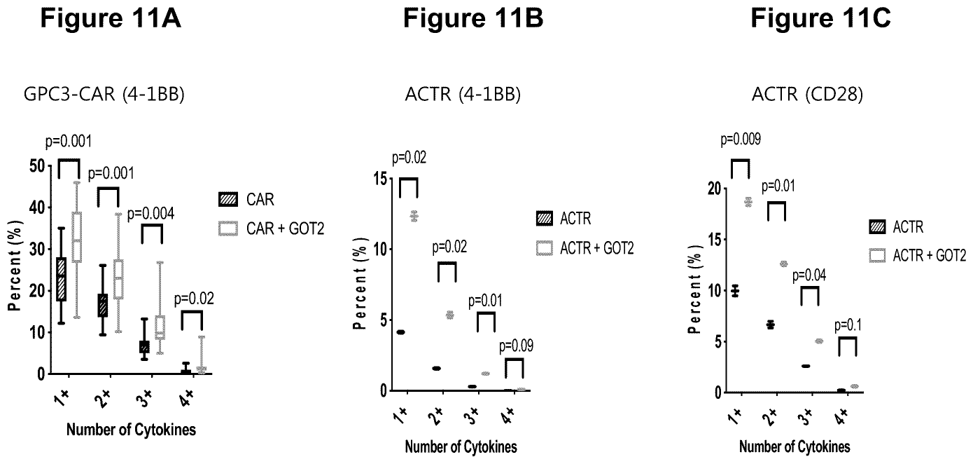

The frequency of CD4+ T cells producing multiple cytokines was quantified by flow cytometry. T cells expressing GOT2 in addition to the CAR or ACTR polypeptides had a higher frequency of CD4+ T cells producing greater than one, two, or three cytokines simultaneously relative to T cells expressing CAR or ACTR alone, demonstrating greater polyfunctionality in the GOT2-expressing cells (FIGS. 11A-11C). These experiments demonstrate that expressing a Krebs cycle modulating polypeptide in T cells has a positive impact on CD4 T cell polyfunctionality.

### Example 16: Co-Expression of a Krebs Cycle Modulating Gene (GOT2) and Either a GPC3-Targeting CAR or ACTR Increases Frequency of CD8+ Cells with Less-Differentiated, Naïve-Like Phenotype

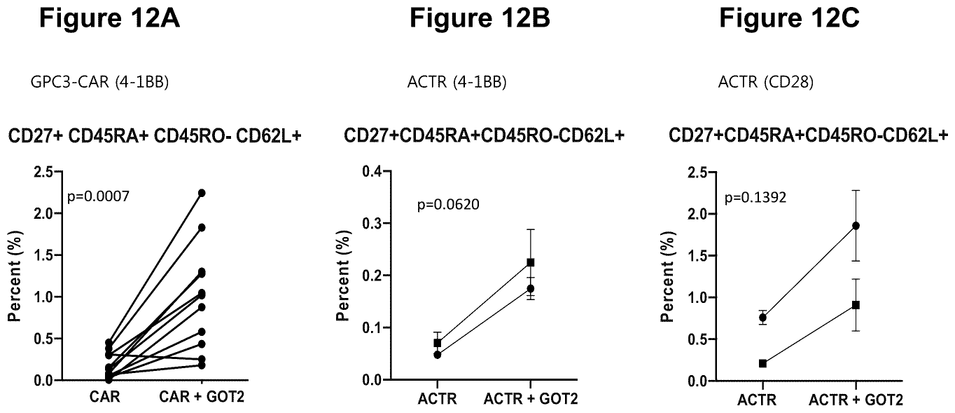

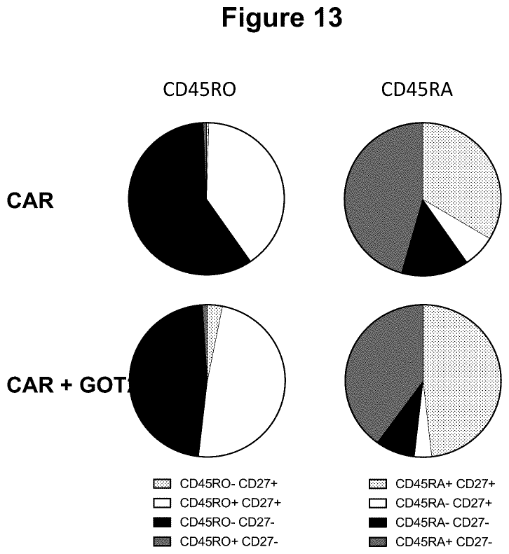

A Krebs cycle modulating polypeptide (GOT2) (SEQ ID NO: 88) was co-expressed in the same T cells with a GPC3-targeting CAR-T polypeptide (SEQ ID NO:104) or was co-expressed in the same T cells with an ACTR polypeptide containing either a 4-1BB (SEQ ID NO: 1) or CD28 (SEQ ID NO:57) primary costimulatory domain. The T cells were transduced with virus encoding the CAR or ACTR polypeptide alone or CAR or ACTR and GOT2 separated by a P2A ribosomal skip sequence. T cell phenotype of CAR constructs from 11 healthy donors and of ACTR constructs for two healthy donors was assessed by flow cytometry. For these experiments, cells were thawed and stained with anti-CD3, anti-CD4, anti-CD8, anti-CD45RA, anti-CD45RO, anti-CD27 and anti-CD62L antibodies for both CAR- and ACTR-expressing T cells. CAR T cells were also stained with recombinant GPC3 protein to detect the CAR polypeptide and ACTR T cells were also stained with an anti-CD16 antibody to detect the ACTR polypeptide. The frequency of CD8+CAR+ or ACTR+ T cells that stained positive for CD27, CD45RA, CD62L and negative for CD45RO was higher in T cells that also co-expressed GOT2 relative to the cognate parent, demonstrating that GOT2-expressing cells have a more naïve-like CD8+ population (FIGS. 12A-12C). Overall, the phenotype of CD8+CAR or ACTR T cells co-expressing GOT2 show a greater proportion of cells with phenotypic markers of less differentiated (younger) phenotype (CD27+/CD45RO+, CD27+/CD45 RO−, CD27+/CD45RA+, CD27+/CD45RA−) relative to parent CAR (FIG. 13). These experiments demonstrate that expressing a Krebs cycle modulating polypeptide in T cells has a positive impact on CD8 phenotype.

### Example 17: Co-Expression of a Krebs Cycle Modulating Gene (GOT2) and a GPC3-Targeting CAR in T Cells Improves Proliferation Under Chronic Antigen Stimulation and Hypoxia

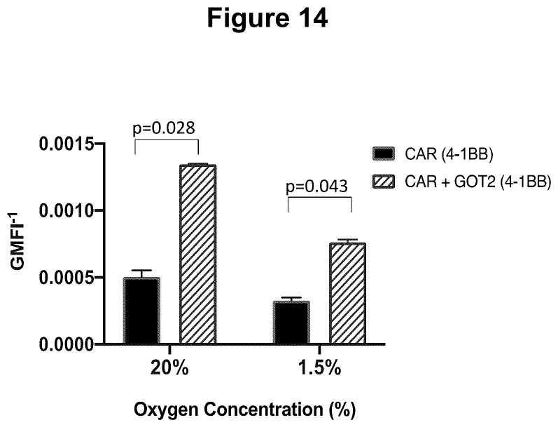

A Krebs cycle modulating polypeptide (GOT2) (SEQ ID NO: 88) was co-expressed in the same T cells with a GPC3-targeting CAR-T polypeptide (SEQ ID NO: 104). The T cells were transduced with virus encoding the CAR polypeptide alone or CAR and GOT2 separated by a P2A ribosomal skip sequence. T cells expressing GPC3-targeting CAR and T cells co-expressing GPC3-CAR and GOT2 cells were labeled with cell trace violet, fluorescently labeling cellular proteins, and then mixed at a 2:1 effector-to-target ratio with GPC3+ hepatocellular carcinoma Hep3B tumor cells. On day 3 after activation, cells were washed, and then mixed at a 2:1 effector-to-target ratio with fresh GPC3+ hepatocellular carcinoma Hep3B tumor cells as a second antigen stimulation. Cells were incubated under normoxic (20% oxygen) or hypoxic (1.5% oxygen) conditions for 3 additional days, and then stained with anti-CD3 and cell trace violet dilution was measured by flow cytometry to measure proliferation, expressed as inverse of cell trace violet mean fluorescence intensity (MFI-1). T cells expressing GOT2 in addition to the CAR polypeptide demonstrated increased proliferation under chronic antigen stimulation, under both the normoxic and hypoxic conditions (FIG. 14), simulating multiple stresses of the tumor microenvironment. These experiments demonstrate that expressing a Krebs cycle modulating polypeptide in T cells provides a proliferative advantage under unfavorable conditions present in the solid tumor microenvironment.

### Example 18: Co-Expression of a Krebs Cycle Modulating Gene (GOT2) and a GPC3-Targeting CAR in T Cells Improves Proliferation in Limiting Glucose Conditions

A Krebs cycle modulating polypeptide (GOT2) (SEQ ID NO: 88) was co-expressed in the same T cells with a GPC3-targeting CAR-T polypeptide containing a 4-1BB (SEQ ID NO: 104) or CD28 (SEQ ID NO: 105) costimulatory domain. The T cells were transduced with virus encoding the CAR polypeptide alone or CAR and GOT2 separated by a P2A ribosomal skip sequence. T cells transduced with the GPC3-targeting CAR-T polypeptide were isolated with AlexaFluor 647-conjugated GPC3 recombinant protein and anti-AlexaFluor647 microbeads (Miltenyi Biotec) and allowed to rest in RPMI+10% FBS overnight. Transduced T cells were then labeled with cell trace violet and mixed at a 2:1 effector-to-target ratio with GPC3+ hepatocellular carcinoma HepG2 tumor cells. Co-cultures were incubated in the presence of high (10 mM) or low (1.25 mM) glucose to mimic the limited nutrient availability in the tumor microenvironment. After 7 days at 37° C. in a 5% CO₂ incubator, the cells were stained with an anti-CD3 antibody and cell division was assessed by cell trace violet dilution via flow cytometry.

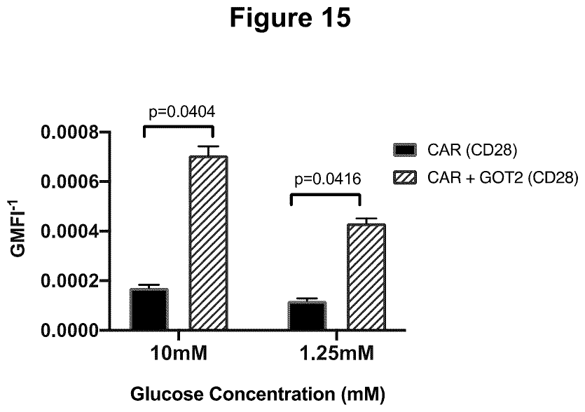

T cells co-expressing GOT2 and the CAR polypeptide demonstrated greater proliferation in both 10 mM and 1.25 mM glucose relative to T cells expressing CAR alone, as indicated by decreased cell trace violet fluorescence intensity. The inverse of cell trace violet fluorescence intensity was plotted as a function of glucose condition and T cell type (FIG. 15). These experiments demonstrate that expressing a Krebs cycle modulating polypeptide in T cells has a positive impact on T cell proliferation in CAR-T cells that contain 4-1BB or CD28 costimulatory domains and in high and low glucose conditions.

### Example 19: Co-Expression of a Krebs Cycle Modulating Gene (GOT2) and a GPC3-Targeting CAR in T Cells Improves Proliferation in the Presence of the T Cell Inhibitor Kynurenine

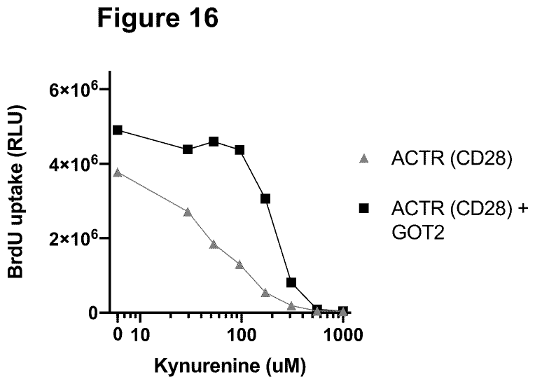

A Krebs cycle modulating polypeptide (GOT2) (SEQ ID NO: 88) was co-expressed in the same T cells with an ACTR polypeptide (SEQ ID NO:57) containing a CD28 primary costimulatory domain and compared in functional assays with T cells expressing the ACTR polypeptide alone. The T cells were transduced with virus encoding the ACTR polypeptide alone or ACTR and GOT2 separated by a P2A ribosomal skip sequence. T cells were mixed at a 4:1 effector-to-target ratio with folate receptor alpha expressing (FOLR+) ovarian carcinoma IGROV-1 tumor cells (fixed in paraformaldehyde) with the addition of anti-FOLR antibody (5 μg/mL). Co-cultures were incubated for 6 days at 37° C. in a 5% CO₂ incubator. Following incubation, half of the cells were transferred to a new plate, pulsed with BrdU (Millipore Sigma), incubated for ˜16 hrs at 37° C. in a 5% CO₂ incubator, and analyzed for BrdU uptake following the manufacturer's directions using an EnVision plate reader (Perkin Elmer) to detect chemiluminescence. T cells co-expressing GOT2 and ACTR demonstrated improved proliferation, as shown by increased BrdU uptake, both in the absence of and in the presence of kynurenine relative to T cells expressing ACTR alone. These experiments demonstrate that expressing a Krebs cycle modulating polypeptide in T cells provides a proliferative advantage in the presence of inhibitory molecules found in the solid tumor microenvironment. FIG. 16.

### Example 20: Co-Expression of a Krebs Cycle Modulating Gene (GOT2) and a GPC3-Targeting CAR in T Cells Results in Diminished Sustained Inhibitory Receptor Expression on T Cells Isolated from GPC3+ Solid Tumor Xenografts

A Krebs cycle modulating polypeptide (GOT2) (SEQ ID NO: 88) was co-expressed in the same T cells with a GPC3-targeting CAR-T polypeptide (SEQ ID NO: 104). The T cells were transduced with virus encoding the CAR polypeptide alone or CAR and GOT2 separated by a P2A ribosomal skip sequence.

The ex vivo phenotype of CAR T cells was evaluated after injection into a mouse tumor model. For these experiments, the GPC3+ hepatocellular carcinoma tumor cell line, JHH7 was inoculated into NSG™ (NOD scid gamma, NOD.Cg-Prkdc^{scid} IL2rg^{tmWj1}/SzJ, Strain 005557) mice. Treatment with T cells was initiated when tumor volumes reached approximately 100 mm³ (day 11 post inoculation). Mice were randomized into treatment groups of 5 mice each based on tumor volume, and treated with T cells expressing the GPC3-targeted CAR or T cells co-expressing the GPC3-targeted CAR and GOT2 at a dose of 5×10⁶ CAR+ T cells on day 11 post inoculation.

Subcutaneous tumors were harvested from JHH7 tumor bearing mice on day 2, 7, and 14 post T cell dosing. Tumors were minced with a razor blade and enzymatically digested using a human tumor dissociation kit (Miltenyi Biotec) for 1 hour at 37° C. Digested tumors were passed through a 70 μm cell strainer prior to seeding in a 96 well plate for cell staining.

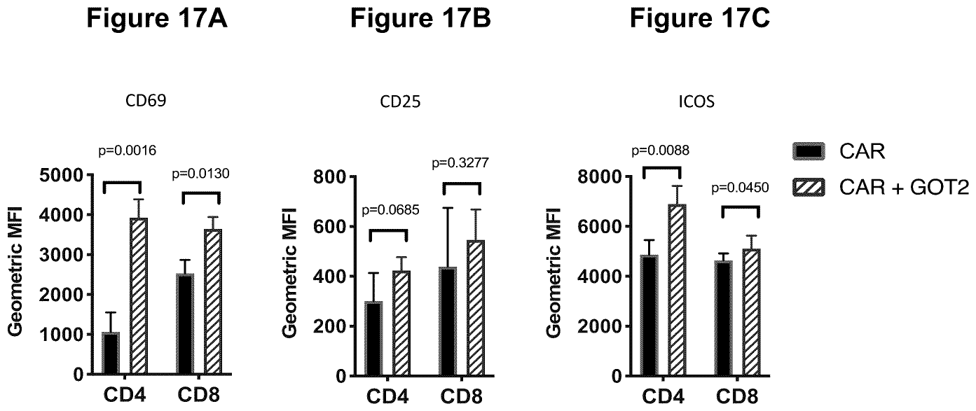

Immunodeficient mice bearing established JHH7 GPC3-expressing subcutaneous tumors were treated with a single intravenous injection of 5×10⁶ T cells co-expressing CAR and GOT2 or T cells expressing CAR alone. Cohorts of animals were euthanized on days 2, 7 and 14 and T cells were isolated from tumors through manual and enzymatic digestion. Flow cytometry was performed, and mean fluorescence intensity of CD69, CD25, and ICOS activation molecules was quantified on CD4+ and CD8+ T cell subsets. T cell phenotype was assessed by flow cytometry following red blood cell lysis and cell staining. For these experiments, cells isolated from tumors were stained with anti-CD3, anti-CD4, anti-CD8, anti-PD-1, anti-TIM-3, anti-CD69, anti-CD25, and anti-ICOS antibodies. The mean fluorescence intensity of CD69, CD25, and ICOS activation molecules were quantified on CD4+ and CD8+ T cell subsets isolated from the tumor on day 2 (CD69) or day 7 (CD25, ICOS) and plotted for each T cell type. As shown in FIGS. 17A-17C, T cells expressing co-expressing GOT2 and the CAR polypeptide demonstrated increased expression of CD69, CD25, and ICOS activation markers relative to T cells expressing the CAR alone.

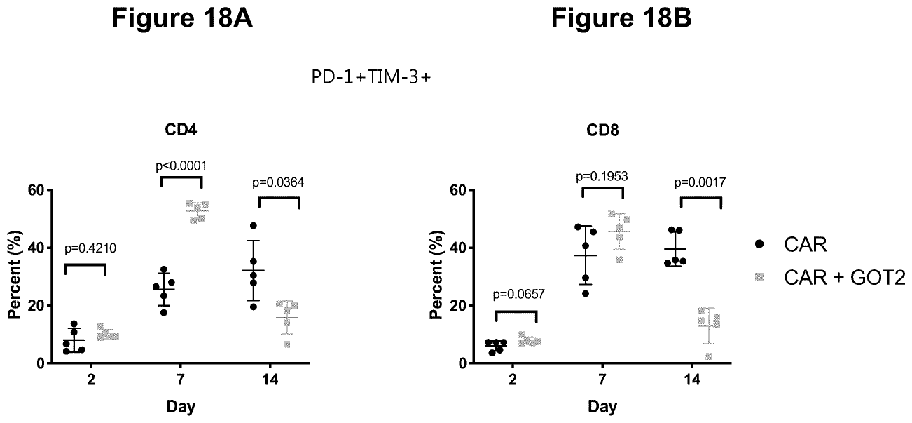

Immunodeficient mice bearing established JHH7 GPC3-expressing subcutaneous tumors were treated with a single intravenous injection of 5×10⁶ T cells co-expressing CAR and GOT2 or T cells expressing CAR alone. Cohorts of animals were euthanized on days 2, 7 and 14 and T cells were isolated from tumors and spleen through manual and enzymatic digestion. Flow cytometry was performed, and the frequency of T cells co-expressing PD-1 and TIM-3 inhibitory molecules was quantified on CD4+ and CD8+ T cell subsets. The frequency of T cells co-expressing PD-1 and TIM-3 inhibitory molecules were quantified on CD4+ and CD8+ T cell subsets isolated from the tumor and plotted for each T cell type as a function of timepoint. T cells co-expressing CAR and GOT2 showed comparable or higher frequency of PD-1+TIM-3+ T cells on day 7 relative to T cells expressing CAR alone, but lower frequency of PD-1+TIM-3+ T cells on day 14. FIGS. 18A-18B. These results show that T cells co-expressing CAR and GOT2 are more resistant to sustained expression of inhibitory receptors relative to T cells expressing CAR alone. These experiments demonstrate that expressing a Krebs cycle modulating polypeptide in T cells enhances activation and limits sustained inhibitory receptor expression of T cell in the solid tumor microenvironment.

### Example 21: Co-Expression of a Krebs Cycle Modulating Gene (GOT2) and a GPC3-Targeting CAR in T Cells Results in Resistance to Sustained Inhibitory Receptor Expression on T Cells Isolated from GPC3+Solid Tumor Xenografts

A Krebs cycle modulating polypeptide (GOT2) (SEQ ID NO: 88) was co-expressed in the same T cells with a GPC3-targeting CAR-T polypeptide (SEQ ID NO:104). The T cells were transduced with virus encoding the CAR polypeptide alone or CAR and GOT2 separated by a P2A ribosomal skip sequence.

The ex vivo phenotype of CAR T cells was evaluated after injection into a mouse tumor model. For these experiments, the GPC3+ hepatocellular carcinoma tumor cell line, JHH7 was inoculated into NSG™ (NOD scid gamma, NOD.Cg-Prkdc^{scid} IL2rg^{tmWj1}/SzJ, Strain 005557) mice. Treatment with T cells was initiated when tumor volumes reached approximately 80 mm³ (day 10 post inoculation). Mice were randomized into treatment groups of 3 mice each based on tumor volume, and treated with T cells expressing the GPC3-targeted CAR or T cells co-expressing the GPC3-targeted CAR and GOT2 at a dose of 5 x 106 CAR+ T cells on day 11 post inoculation.

Subcutaneous tumors and spleen tissues were harvested from JHH7 tumor bearing mice on day 1, 6, and 13 post T cell dosing. Tumors were minced with a razor blade and enzymatically digested using a human tumor dissociation kit (Miltenyi Biotec) for 1 hour at 37° C. Spleens were dissociated mechanically using the end of a syringe plunger. Digested tumors and spleens were passed through a 70 μm cell strainer prior to seeding in a 96 well plate for cell staining.

Immunodeficient mice bearing established JHH7 GPC3-expressing subcutaneous tumors were treated with a single intravenous injection of 5×10⁶ T cells co-expressing CAR and GOT2 or T cells expressing CAR alone. Cohorts of animals were euthanized on days 6 and 13, and T cells were isolated from tumors and spleen through manual and enzymatic digestion. Flow cytometry was performed, and the frequency of T cells co-expressing PD-1 and TIM-3 inhibitory molecules was quantified on CD4+ and CD8+ T cell subsets.

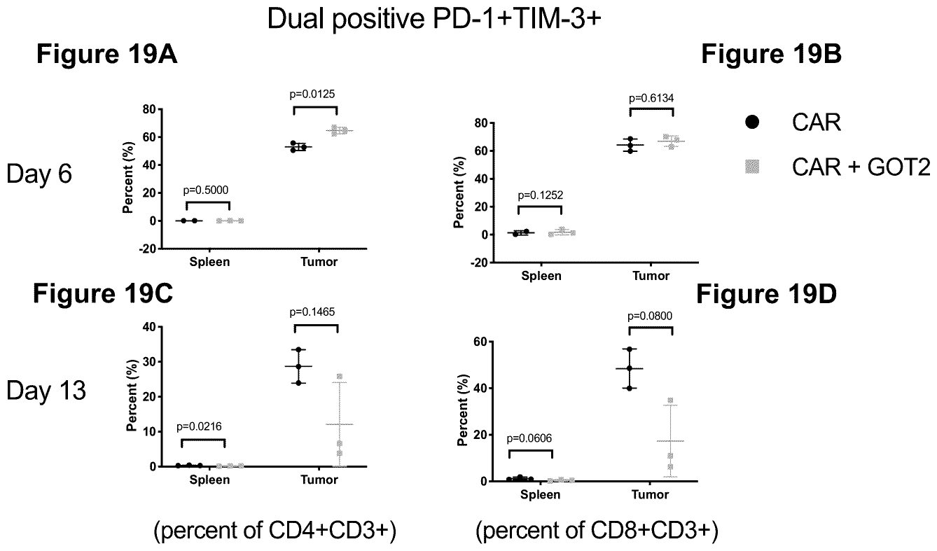

T cell phenotype was assessed by flow cytometry following red blood cell lysis and cell staining. For these experiments, cells were stained with anti-CD3, anti-CD4, anti-CD8, anti-PD-1, and anti-TIM-3 antibodies. The frequency of T cells co-expressing PD-1 and TIM-3 was quantified on CD4+ and CD8+ T cell subsets. T cells in CD4+ and CD8+ subsets co-expressing CAR and GOT2 demonstrated comparable to higher frequency of PD-1+TIM-3+ T cells on day 6 relative to T cells expressing CAR alone, indicating activation, but a diminished frequency of expression after prolonged exposure to the tumor microenvironment (day 13) compared T cells expressing CAR alone, indicating a resistance to sustained inhibitory receptor expression. T cells isolated from the spleen did not show PD-1+TIM-3+ expression at either day 6 or 13, demonstrating the specificity of activation to GPC3-expressing tumor only. FIGS. 19A-19D. T cells co-expressing CAR and GOT2 showed higher frequency of PD-1+TIM-3+ T cells on day 6 relative to T cells expressing CAR alone. FIGS. 19A and 18B. On the other hand, T cells co-expressing CAR and GOT2 showed lower frequency of PD-1+TIM-3+ T cells on day 13, after prolonged exposure to the tumor microenvironment, indicating a resistance to sustained inhibitory receptor expression, compared to T cells expressing CAR alone. FIGS. 19C and 19D. T cells isolated from the spleen did not show PD-1+TIM-3+ expression at either day 6 or 13, demonstrating the specificity of T cell activation in GPC3-expressing tumor only. FIGS. 19A-19D.

These experiments demonstrate that expressing a Krebs cycle modulating polypeptide in T cells provides resistance sustained inhibitory receptor expression in the solid tumor microenvironment.

### Example 22: Co-Expression of a Krebs Cycle Modulating Gene (GOT2) and a GPC3-Targeting CAR in T Cells Results in Sustained T Cell Functionality after Exposure to the Tumor Microenvironment In Vivo

A Krebs cycle modulating polypeptide (GOT2) (SEQ ID NO: 88) was co-expressed in the same T cells with a GPC3-targeting CAR-T polypeptide (SEQ ID NO: 104). The T cells were transduced with virus encoding the CAR polypeptide alone or CAR and GOT2 separated by a P2A ribosomal skip sequence.

The function of CAR transduced cells was evaluated in a mouse tumor model. For these experiments, the GPC3+ hepatocellular carcinoma tumor cell line, JHH7 was inoculated into NSG™ (NOD scid gamma, NOD.Cg-Prkdc^{scid} IL2rg^{tmWj1}/SzJ, Strain 005557) mice. Treatment with T cells was initiated when tumor volumes reached approximately 80 mm³ (day 10 post inoculation). Mice were randomized into treatment groups of 3 mice each based on tumor volume, and treated with T cells expressing the GPC3-targeted CAR at a dose of 5×10⁶ CAR+ T cells on day 11 post inoculation.

Subcutaneous tumors were harvested from JHH7 tumor bearing mice on day 3 post CAR dose. Tumors were minced with a razor blade and enzymatically digested using a human tumor dissociation kit (Miltenyi Biotec) for 1 hour at 37° C. Digested tumors were passed through a 70 μm cell strainer prior to seeding in a 24 well plate (1×10⁶ cells/1 mL volume). Following incubation for 18 hours at 37° C. in a 5% CO₂ incubator, cell supernatants were collected for cytokine analysis. The concentration of IFNγ and IL-17A in the cell supernatants was determined using homogenous time resolved fluorescence assay (Cisbio) and Meso Scale Discovery V-Plex assay technology, respectively. Both assays were performed according to the manufacturer's protocol.

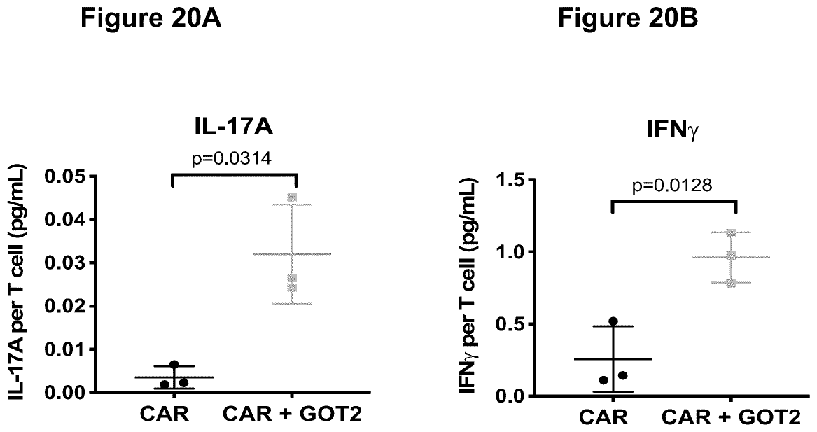

T cells within tumors from mice dosed with T cells co-expressing GOT2 and CAR demonstrated increased IFNγ and IL-17A production relative to T cells within tumors from mice dosed with T cells expressing CAR alone. FIGS. 20A-20B. These experiments demonstrate that expressing a Krebs cycle modulating polypeptide in T cells has a positive impact on T cell function in the tumor microenvironment.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1-103. (canceled)

104. A genetically engineered T cell, which expresses:
(a) a glutamic-oxaloacetic transaminase (GOT), which is encoded by an exogenous nucleic acid; and
(b) a chimeric receptor polypeptide comprising:
(i) an extracellular target binding domain,
(ii) a transmembrane domain, and
(iii) a cytoplasmic signaling domain.

105. The genetically engineered T cell of claim 104, wherein the chimeric receptor polypeptide further comprises a co-stimulatory signaling domain.

106. The genetically engineered T cell of claim 105, wherein the co-stimulatory domain is of 4-1BB or CD28.

107. The genetically engineered T cell of claim 104, wherein the cytoplasmic signaling domain of (c) is a cytoplasmic domain of CD3ζ or FcεRIγ.

108. The genetically engineered T cell of claim 104, wherein the chimeric receptor polypeptide further comprises a hinge domain, which is located at the C-terminus of (a) and the N-terminus of (b).

109. The genetically engineered T cell of claim 104, wherein the chimeric receptor polypeptide further comprises a signal peptide at its N-terminus.

110. The genetically engineered T cell of claim 104, wherein the chimeric receptor polypeptide is a chimeric antigen receptor (CAR) polypeptide and the extracellular target binding domain thereof comprises a single chain antibody fragment.

111. The genetically engineered T cell of claim 110, wherein the CAR polypeptide comprises, from the N-terminus to the C-terminus, (i) the extracellular target binding domain, (ii) a transmembrane domain, (iii) a hinge domain, (iv) a co-stimulatory domain, and (v) a cytoplasmic signaling domain.

112. The genetically engineered T cell of claim 110, wherein:
(a) the transmembrane domain and the hinge domain are from CD28, the co-stimulatory signaling domain is from CD28, and the cytoplasmic signaling domain is from CD3ζ; or
(b) the transmembrane domain and the hinge domain are from CD8a, the co-stimulatory signaling domain is from 4-1BB, and the cytoplasmic signaling domain is from CD3ζ.

113. The genetically engineered T cell of claim 112, wherein the wherein the CAR polypeptide comprises the amino acid sequence of SEQ ID NOs: 104 or 105.

114. The genetically engineered T cell of claim 104, wherein the GOT is GOT1 or GOT2.

115. The genetically engineered T cell of claim 114, wherein the GOT is GOT2.

116. The genetically engineered T cell of claim 115, wherein the chimeric receptor polypeptide is a CAR polypeptide comprising the amino acid sequence of SEQ ID NOs: 104 or 105.

117. The genetically engineered T cell of claim 116, wherein in the T cell, the expression of an endogenous T cell receptor, an endogenous major histocompatibility complex, an endogenous beta-2-microglobulin, or a combination thereof has been inhibited or eliminated.

118. The genetically engineered T cell of claim 104, wherein the genetically modified T cell comprises a nucleic acid or nucleic acid set, which collectively comprises:
(a) a first nucleotide sequence encoding the GOT polypeptide; and
(b) a second nucleotide sequence encoding the chimeric receptor polypeptide.

119. The genetically engineered T cell of claim 118, wherein the nucleic acid further comprises a third nucleotide sequence located between the first nucleotide sequence and the second nucleotide sequence, wherein the third nucleotide sequence encodes a ribosomal skipping site, an internal ribosome entry site (IRES), or a second promoter.

120. The genetically engineered T cell of claim 119, wherein the third nucleotide sequence encodes a ribosomal skipping site, which is a P2A peptide.

121. A population of immune cells, comprising a plurality of the genetically engineered T cells of claim 104.

122. A method for treating liver cancer, comprising administering to a subject in need thereof an effective amount of a population of immune cells comprising the genetically engineered T cell of claim 104, wherein the genetically engineered T cell expresses the chimeric receptor polypeptide, which is a CAR polypeptide comprising the amino acid sequence of SEQ ID NO: 104 or 105.

123. The method of claim 122, wherein the population of immune cells is autologous to the human patient.


SEQUENCE LISTING (107 sequences)

SEQ ID NO: 1 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 442 aa, synthetic construct
  REGION 1..442: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIISFFLALTSTALLFLLFFLTLRFSVVKRGKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 442 aa, synthetic construct
  REGION 1..442: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDFWVLVVVGGVLACYSLLVTVAFIIFWVRSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLIALVTSGALLAVLGITGYFLMNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLLAALLALLAALLALLAALLARSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 428 aa, synthetic construct
  REGION 1..428: note = Synthetic polypeptide
MALPVTALLLPLALLLHAARPQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 435 aa, synthetic construct
  REGION 1..435: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCALYLLRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 477 aa, synthetic construct
  REGION 1..477: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 391 aa, synthetic construct
  REGION 1..391: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEPSEGSAPGSPAGSPTIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 435 aa, synthetic construct
  REGION 1..435: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 477 aa, synthetic construct
  REGION 1..477: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 437 aa, synthetic construct
  REGION 1..437: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDMALIVLGGVAGLLLFIGLGIFFCVRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDMALIVLGGVAGLLLFIGLGIFFCVRRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLCYILDAILFLYGIVLTLLYCRLKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLLLILLGVLAGVLATLAALLARSKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDITLGLLVAGVLVLLVSLGVAIHLCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVSFCLVMVLLFAVDTGLYFSVKTNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVAAILGLGLVLGLLGPLAILLALYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLCYLLDGILFIYGVILTALFLRVKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 441 aa, synthetic construct
  REGION 1..441: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVMSVATIVIVDICITGGLLLLVYYWSKNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDGFLFAEIVSIFVLAVGVYFIAGQDKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 437 aa, synthetic construct
  REGION 1..437: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDGIIVTDVIATLLLALGVFCFAGHETKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 435 aa, synthetic construct
  REGION 1..435: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVIGFRILLLKVAGFNLLMTLRLWKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 438 aa, synthetic construct
  REGION 1..438: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIIVAVVIATAVAAIVAAVVALIYCRKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDVLFYLAVGIMFLVNTVLWVTIRKEKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIIILVGTAVIAMFFWLLLVIILRTKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 432 aa, synthetic construct
  REGION 1..432: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDLGWLCLLLLPIPLIVWVKRKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIAIYCIGVFLIACMVVTVILCRMKKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 476 aa, synthetic construct
  REGION 1..476: note = Synthetic polypeptide
MALPVTALLLPLALLLHAARPQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 623 aa, synthetic construct
  REGION 1..623: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 510 aa, synthetic construct
  REGION 1..510: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 403 aa, synthetic construct
  REGION 1..403: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQEPKSCDKTHTCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 421 aa, synthetic construct
  REGION 1..421: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEAFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 406 aa, synthetic construct
  REGION 1..406: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 406 aa, synthetic construct
  REGION 1..406: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 421 aa, synthetic construct
  REGION 1..421: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 436 aa, synthetic construct
  REGION 1..436: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 451 aa, synthetic construct
  REGION 1..451: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGGGSGGGGSGGGGSGGGGSGGGGSGGGGSGGGGSGGGGSGGGGSGGGGSGGGGSGGGGSIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 451 aa, synthetic construct
  REGION 1..451: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEPSEGSAPGSPAGSPTSTEEGTSTEPSEGSAIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 421 aa, synthetic construct
  REGION 1..421: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTSESATPESGPGTSTEIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 406 aa, synthetic construct
  REGION 1..406: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGGSPAGSPTSTEEGTIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - protein, 433 aa, synthetic construct
  REGION 1..433: note = Synthetic polypeptide
MLRLLLALNLFPSIQVTGGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - protein, 436 aa, synthetic construct
  REGION 1..436: note = Synthetic polypeptide
METDTLLLWVLLLWVPGSTGDGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 431 aa, synthetic construct
  REGION 1..431: note = Synthetic polypeptide
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 432 aa, synthetic construct
  REGION 1..432: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 442 aa, synthetic construct
  REGION 1..442: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCQRRKYRSNKGESPVEPAEPCRYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 452 aa, synthetic construct
  REGION 1..452: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCQLGLHIWQLRSQCMWPRETQLLLEVPPSTEDARSCQFPEEERGERSAEEKGRLGDLWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 454 aa, synthetic construct
  REGION 1..454: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCCVKRRKPRGDVVKVIVSVQRKRQEAEGEATVIEALQAPPDVTTVAVEETIPSFTGRSPNHRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 442 aa, synthetic construct
  REGION 1..442: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKKYFFKKEVQQLSVSFSSLQIKALQNAVEKEVQAEDNIYIENSLYATDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 453 aa, synthetic construct
  REGION 1..453: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCYKVGFFKRNLKEKMEAGRGVPNGIPAEDSEQLASGQEAGDPGCLKPLHEKDSESGGGKDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 510 aa, synthetic construct
  REGION 1..510: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 365 aa, synthetic construct
  REGION 1..365: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDPQLCYILDAILFLYGIVLTLLYCRLKIQVRKAAITSYEKSDGVYTGLSTRNQETYETLKHEKPPQKRGRKKLLYIFKQPFMRPVQTTQEEDGCSCRFPEEEEGGCEL

SEQ ID NO: 56 - protein, 366 aa, synthetic construct
  REGION 1..366: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRLKIQVRKAAITSYEKSDGVYTGLSTRNQETYETLKHEKPPQ

SEQ ID NO: 57 - protein, 432 aa, synthetic construct
  REGION 1..432: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 390 aa, synthetic construct
  REGION 1..390: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 483 aa, synthetic construct
  REGION 1..483: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSQRRKYRSNKGESPVEPAEPCHYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 472 aa, synthetic construct
  REGION 1..472: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 477 aa, synthetic construct
  REGION 1..477: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 474 aa, synthetic construct
  REGION 1..474: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 433 aa, synthetic construct
  REGION 1..433: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 442 aa, synthetic construct
  REGION 1..442: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCQRRKYRSNKGESPVEPAEPCHYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 435 aa, synthetic construct; identical to SEQ ID NO: 7
  REGION 1..435: note = Synthetic polypeptide

SEQ ID NO: 66 - protein, 428 aa, synthetic construct
  REGION 1..428: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 431 aa, synthetic construct
  REGION 1..431: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 469 aa, synthetic construct
  REGION 1..469: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 391 aa, synthetic construct; identical to SEQ ID NO: 10
  REGION 1..391: note = Synthetic polypeptide

SEQ ID NO: 70 - protein, 397 aa, synthetic construct
  REGION 1..397: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCQRRKYRSNKGESPVEPAEPCHYSCPREEEGSTIPIQEDYRKPEPACSPRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 383 aa, synthetic construct
  REGION 1..383: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 386 aa, synthetic construct
  REGION 1..386: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 395 aa, synthetic construct
  REGION 1..395: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 394 aa, synthetic construct
  REGION 1..394: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 438 aa, synthetic construct
  REGION 1..438: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 419 aa, synthetic construct
  REGION 1..419: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 409 aa, synthetic construct
  REGION 1..409: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 393 aa, synthetic construct
  REGION 1..393: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 436 aa, synthetic construct; identical to SEQ ID NO: 1
  REGION 1..436: note = Synthetic polypeptide

SEQ ID NO: 80 - protein, 429 aa, synthetic construct
  REGION 1..429: note = Synthetic polypeptide
MALPVTALLLPLALLLHAARPGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQIEVMYPPPYLDNEKSNGTIIHVKGKHLCPSPLFPGPSKPIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 414 aa, Homo sapiens
MSKKISGGSVVEMQGDEMTRIIWELIKEKLIFPYVELDLHSYDLGIENRDATNDQVTKDAAEAIKKHNVGVKCATITPDEKRVEEFKLKQMWKSPNGTIRNILGGTVFREAIICKNIPRLVSGWVKPIIIGRHAYGDQYRATDFVVPGPGKVEITYTPSDGTQKVTYLVHNFEEGGGVAMGMYNQDKSIEDFAHSSFQMALSKGWPLYLSTKNTILKKYDGRFKDIFQEIYDKQYKSQFEAQKIWYEHRLIDDMVAQAMKSEGGFIWACKNYDGDVQSDSVAQGYGSLGMMTSVLVCPDGKTVEAEAAHGTVTRHYRMYQKGQETSTNPIASIFAWTRGLAHRAKLDNNKELAFFANALEEVSIETIEAGFMTKDLAACIKGLPNVQRSDYLNTFEFMDKLGENLKIKLAQAKL

SEQ ID NO: 82 - protein, 452 aa, Homo sapiens
MAGYLRVVRSLCRASGSRPAWAPAALTAPTSQEQPRRHYADKRIKVAKPVVEMDGDEMTRIIWQFIKEKLILPHVDIQLKYFDLGLPNRDQTDDQVTIDSALATQKYSVAVKCATITPDEARVEEFKLKKMWKSPNGTIRNILGGTVFREPIICKNIPRLVPGWTKPITIGRHAHGDQYKATDFVADRAGTFKMVFTPKDGSGVKEWEVYNFPAGGVGMGMYNTDESISGFAHSCFQYAIQKKWPLYMSTKNTILKAYDGRFKDIFQEIFDKHYKTDFDKNKIWYEHRLIDDMVAQVLKSSGGFVWACKNYDGDVQSDILAQGFGSLGLMTSVLVCPDGKTIEAEAAHGTVTRHYREHQKGRPTSTNPIASIFAWTRGLEHRGKLDGNQDLIRFAQMLEKVCVETVESGAMTKDLAGCIHGLSNVKLNEHFLNTTDFLDTIKSNLDRALGRQ

SEQ ID NO: 83 - protein, 334 aa, Homo sapiens
MSEPIRVLVTGAAGQIAYSLLYSIGNGSVFGKDQPIILVLLDITPMMGVLDGVLMELQDCALPLLKDVIATDKEDVAFKDLDVAILVGSMPRREGMERKDLLKANVKIFKSQGAALDKYAKKSVKVIVVGNPANTNCLTASKSAPSIPKENFSCLTRLDHNRAKAQIALKLGVTANDVKNVIIWGNHSSTQYPDVNHAKVKLQGKEVGVYEALKDDSWLKGEFVTTVQQRGAAVIKARKLSSAMSAAKAICDHVRDIWFGTPEGEFVSMGVISDGNSYGVPDDLLYSFPVVIKNKTWKFVEGLPINDFSREKMDLTAKELTEEKESAFEFLSSA

SEQ ID NO: 84 - protein, 338 aa, Homo sapiens
MLSALARPASAALRRSFSTSAQNNAKVAVLGASGGIGQPLSLLLKNSPLVSRLTLYDIAHTPGVAADLSHIETKAAVKGYLGPEQLPDCLKGCDVVVIPAGVPRKPGMTRDDLFNTNATIVATLTAACAQHCPEAMICVIANPVNSTIPITAEVFKKHGVYNPNKIFGVTTLDIVRANTFVAELKGLDPARVNVPVIGGHAGKTIIPLISQCTPKVDFPQDQLTALTGRIQEAGTEVVKAKAGAGSATLSMAYAGARFVFSLVDAMNGKEGVVECSFVKSQETECTYFSTPLLLGKKGIEKNLGIGKVSSFEEKMISDAIPELKASIKKGEDFVKTLK

SEQ ID NO: 85 - protein, 533 aa, Homo sapiens
MAFANLRKVLISDSLDPCCRKILQDGGLQVVEKQNLSKEELIAELQDCEGLIVRSATKVTADVINAAEKLQVVGRAGTGVDNVDLEAATRKGILVMNTPNGNSLSAAELTCGMIMCLARQIPQATASMKDGKWERKKFMGTELNGKTLGILGLGRIGREVATRMQSFGMKTIGYDPIISPEVSASFGVQQLPLEEIWPLCDFITVHTPLLPSTTGLLNDNTFAQCKKGVRVVNCARGGIVDEGALLRALQSGQCAGAALDVFTEEPPRDRALVDHENVISCPHLGASTKEAQSRCGEEIAVQFVDMVKGKSLTGVVNAQALTSAFSPHTKPWIGLAEALGTLMRAWAGSPKGTIQVITQGTSLKNAGNCLSPAVIVGLLKEASKQADVNLVNAKLLVKEAGLNVTTSHSPAAPGEQGFGECLLAVALAGAPYQAVGLVQGTTPVLQGLNGAVFRPEVPLRRDLPLLLFRTQTSDPAMLPTMIGLLAEAGVRLLSYQTSLVSDGETWHVMGISSLLPSLEAWKQHVTEAFQFHF

SEQ ID NO: 86 - protein, 622 aa, Homo sapiens
MPPQLQNGLNLSAKVVQGSLDSLPQAVREFLENNAELCQPDHIHICDGSEEENGRLLGQMEEEGILRRLKKYDNCWLALTDPRDVARIESKTVIVTQEQRDTVPIPKTGLSQLGRWMSEEDFEKAFNARFPGCMKGRTMYVIPFSMGPLGSPLSKIGIELTDSPYVVASMRIMTRMGTPVLEAVGDGEFVKCLHSVGCPLPLQKPLVNNWPCNPELTLIAHLPDRREIISFGSGYGGNSLLGKKCFALRMASRLAKEEGWLAEHMLILGITNPEGEKKYLAAAFPSACGKTNLAMMNPSLPGWKVECVGDDIAWMKFDAQGHLRAINPENGFFGVAPGTSVKTNPNAIKTIQKNTIFTNVAETSDGGVYWEGIDEPLASGVTITSWKNKEWSSEDGEPCAHPNSRFCTPASQCPIIDAAWESPEGVPIEGIIFGGRRPAGVPLVYEALSWQHGVFVGAAMRSEATAAAEHKGKIIMHDPFAMRPFFGYNFGKYLAHWLSMAQHPAAKLPKIFHVNWFRKDKEGKFLWPGFGENSRVLEWMFNRIDGKASTKLTPIGYIPKEDALNLKGLGHINMMELFSISKEFWEKEVEDIEKYLEDQVNADLPCEIEREILALKQRISQM

SEQ ID NO: 87 - protein, 413 aa, Homo sapiens
MAPPSVFAEVPQAQPVLVFKLTADFREDPDPRKVNLGVGAYRTDDCHPWVLPVVKKVEQKIANDNSLNHEYLPILGLAEFRSCASRLALGDDSPALKEKRVGGVQSLGGTGALRIGADFLARWYNGTNNKNTPVYVSSPTWENHNAVFSAAGFKDIRSYRYWDAEKRGLDLQGFLNDLENAPEFSIVVLHACAHNPTGIDPTPEQWKQIASVMKHRFLFPFFDSAYQGFASGNLERDAWAIRYFVSEGFEFFCAQSFSKNFGLYNERVGNLTVVGKEPESILQVLSQMEKIVRITWSNPPAQGARIVASTLSNPELFEEWTGNVKTMADRILTMRSELRARLEALKTPGTWNHITDQIGMFSFTGLNPKQVEYLVNEKHIYLLPSGRINVSGLTTKNLDYVATSIHEAVTKIQ

SEQ ID NO: 88 - protein, 430 aa, Homo sapiens
MALLHSGRVLPGIAAAFHPGLAAAASARASSWWTHVEMGPPDPILGVTEAFKRDTNSKKMNLGVGAYRDDNGKPYVLPSVRKAEAQIAAKNLDKEYLPIGGLAEFCKASAELALGENSEVLKSGRFVTVQTISGTGALRIGASFLQRFFKFSRDVFLPKPTWGNHTPIFRDAGMQLQGYRYYDPKTCGFDFTGAVEDISKIPEQSVLLLHACAHNPTGVDPRPEQWKEIATVVKKRNLFAFFDMAYQGFASGDGDKDAWAVRHFIEQGINVCLCQSYAKNMGLYGERVGAFTMVCKDADEAKRVESQLKILIRPMYSNPPLNGARIAAAILNTPDLRKQWLQEVKVMADRIIGMRTQLVSNLKKEGSTHNWQHITDQIGMFCFTGLKPEQVERLIKEFSIYMTKDGRISVAGVTSSNVGYLAHAIHQVTK

SEQ ID NO: 89 - protein, 496 aa, Homo sapiens
MASSTGDRSQAVRHGLRAKVLTLDGMNPRVRRVEYAVRGPIVQRALELEQELRQGVKKPFTEVIRANIGDAQAMGQRPITFLRQVLALCVNPDLLSSPNFPDDAKKRAERILQACGGHSLGAYSVSSGIQLIREDVARYIERRDGGIPADPNNVFLSTGASDAIVTVLKLLVAGEGHTRTGVLIPIPQYPLYSATLAELGAVQVDYYLDEERAWALDVAELHRALGQARDHCRPRALCVINPGNPTGQVQTRECIEAVIRFAFEERLFLLADEVYQDNVYAAGSQFHSFKKVLMEMGPPYAGQQELASFHSTSKGYMGECGFRGGYVEVVNMDAAVQQQMLKLMSVRLCPPVPGQALLDLVVSPPAPTDPSFAQFQAEKQAVLAELAAKAKLTEQVFNEAPGISCNPVQGAMYSFPRVQLPPRAVERAQELGLAPDMFFCLRLLEETGICVVPGSGFGQREGTYHFRMTILPPLEKLRLLLEKLSRFHAKFTLEYS

SEQ ID NO: 90 - protein, 669 aa, Homo sapiens
MMRLRGSGMLRDLLLRSPAGVSATLRRAQPLVTLCRRPRGGGRPAAGPAAAARLHPWWGGGGWPAEPLARGLSSSPSEILQELGKGSTHPQPGVSPPAAPAAPGPKDGPGETDAFGNSEGKELVASGENKIKQGLLPSLEDLLFYTIAEGQEKIPVHKFITALKSTGLRTSDPRLKECMDMLRLTLQTTSDGVMLDKDLFKKCVQSNIVLLTQAFRRKFVIPDFMSFTSHIDELYESAKKQSGGKVADYIPQLAKFSPDLWGVSVCTVDGQRHSTGDTKVPFCLQSCVKPLKYAIAVNDLGTEYVHRYVGKEPSGLRFNKLFLNEDDKPHNPMVNAGAIVVTSLIKQGVNNAEKFDYVMQFLNKMAGNEYVGFSNATFQSERESGDRNFAIGYYLKEKKCFPEGTDMVGILDFYFQLCSIEVTCESASVMAATLANGGFCPITGERVLSPEAVRNTLSLMHSCGMYDFSGQFAFHVGLPAKSGVAGGILLVVPNVMGMMCWSPPLDKMGNSVKGIHFCHDLVSLCNFHNYDNLRHFAKKLDPRREGGDQRVKSVINLLFAAYTGDVSALRRFALSAMDMEQRDYDSRTALHVAAAEGHVEVVKFLLEACKVNPFPKDRWNNTPMDEALHFGHHDVFKILQEYQVQYTPQGDSDNGKENQTVHKNLDGLL

SEQ ID NO: 91 - protein, 370 aa, Homo sapiens
MDAPRQVVNFGPGPAKLPHSVLLEIQKELLDYKGVGISVLEMSHRSSDFAKIINNTENLVRELLAVPDNYKVIFLQGGGCGQFSAVPLNLIGLKAGRCADYVVTGAWSAKAAEEAKKFGTINIVHPKLGSYTKIPDPSTWNLNPDASYVYYCANETVHGVEFDFIPDVKGAVLVCDMSSNFLSKPVDVSKFGVIFAGAQKNVGSAGVTVVIVRDDLLGFALRECPSVLEYKVQAGNSSLYNTPPCFSIYVMGLVLEWIKNNGGAAAMEKLSSIKSQTIYEIIDNSQGFYVCPVEPQNRSKMNIPFRIGNAKGDDALEKRFLDKALELNMLSLKGHRSVGGIRASLYNAVTIEDVQKLAAFMKKFLEMHQL

SEQ ID NO: 92 - protein, 391 aa, Homo sapiens
MTYKCAVVDVPFGGAKAGVKINPKNYTDNELEKITRRFTMELAKKGFIGPGIDVPAPDMSTGEREMSWIADTYASTIGHYDINAHACVTGKPISQGGIHGRISATGRGVFHGIENFINEASYMSILGMTPGFGDKTFVVQGFGNVGLHSMRYLHRFGAKCIAVGESDGSIWNPDGIDPKELEDFKLQHGSILGFPKAKPYEGSILEADCDILIPAASEKQLTKSNAPRVKAKIIAEGANGPTTPEADKIFLERNIMVIPDLYLNAGGVTVSYFEWLKNLNHVSYGRLTFKYERDSNYHLLMSVQESLERKFGKHGGTIPIVPTAEFQDRISGASEKDIVHSGLAYTMERSARQIMRTAMKYNLGLDLRTAAYVNAIEKVFKVYNEAGVTFT

SEQ ID NO: 93 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 94 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 95 - protein, 300 aa, synthetic construct
  REGION 1..300: note = Synthetic polypeptide
  REGION 16..300: note = MISC_FEATURE - these amino acids may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 96 - protein, 15 aa, synthetic construct
  REGION 1..15: note = Synthetic polypeptide
GGGGSGGGGSGGGGS

SEQ ID NO: 97 - protein, 30 aa, synthetic construct
  REGION 1..30: note = Synthetic polypeptide
GGGGSGGGGSGGGGSGGGGSGGGGSGGGGS

SEQ ID NO: 98 - protein, 45 aa, synthetic construct
  REGION 1..45: note = Synthetic polypeptide
GGGGSGGGGSGGGGSGGGGSGGGGSGGGGSGGGGSGGGGSGGGGS

SEQ ID NO: 99 - protein, 60 aa, synthetic construct
  REGION 1..60: note = Synthetic polypeptide
GGGGSGGGGSGGGGSGGGGSGGGGSGGGGSGGGGSGGGGSGGGGSGGGGSGGGGSGGGGS

SEQ ID NO: 100 - protein, 75 aa, synthetic construct
  REGION 1..75: note = Synthetic polypeptide
GGGGSGGGGSGGGGSGGGGSGGGGSGGGGSGGGGSGGGGSGGGGSGGGGSGGGGSGGGGSGGGGSGGGGSGGGGS

SEQ ID NO: 101 - protein, 150 aa, synthetic construct
  REGION 1..150: note = Synthetic polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 102 - protein, 225 aa, synthetic construct
  REGION 1..225: note = Synthetic polypeptide
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 103 - protein, 300 aa, synthetic construct; identical to SEQ ID NO: 95
  REGION 1..300: note = Synthetic polypeptide

SEQ ID NO: 104 - protein, 487 aa, synthetic construct
  REGION 1..487: note = Synthetic polypeptide
MALPVTALLLPLALLLHAARPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - protein, 483 aa, synthetic construct
  REGION 1..483: note = Synthetic polypeptide
MALPVTALLLPLALLLHAARPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 19 aa, synthetic construct
  REGION 1..19: note = Synthetic polypeptide
ATNFSLLKQAGDVEENPGP

SEQ ID NO: 107 - protein, 18 aa, synthetic construct
  REGION 1..18: note = Synthetic polypeptide
ATNFSLLKQAGDVEENPG